(19) United States
(12) Patent Application Publication
(10) Pub. No.: US 2023/0086722 A1
(43) Pub. Date: Mar. 23, 2023

(54) METHODS FOR ANALYSIS OF TARGET MOLECULES IN BIOLOGICAL FLUIDS

(71) Applicant: GRAIL, LLC, Menlo Park, CA (US)

(72) Inventors: Matthew LARSON, Amherst, MA (US); Ruth E. MAUNTZ, Redwood, CA (US); David BURKHARDT, San Mateo, CA (US)

(21) Appl. No.: 17/931,016

(22) Filed: Sep. 9, 2022

Related U.S. Application Data
(60) Provisional application No. 63/242,872, filed on Sep. 10, 2021.

(51) Int. Cl. G01N 33/574; G01N 33/68

(52) U.S. Cl. CPC G01N 33/57423; G01N 33/6893; G01N 33/57415; G01N 2800/7028

(57) ABSTRACT

Methods for measuring subpopulations of target molecules (e.g., polypeptides and/or cell-free ribonucleic acid) are provided. In some embodiments, methods of generating a sequencing library from a plurality of RNA molecules in a test sample obtained from a subject are provided, as well as methods for analyzing the sequencing library to detect, e.g., the presence or absence of a disease.

### CROSS-REFERENCE

This application claims the benefit of U.S. Provisional Application No. 63/242,872, filed Sep. 10, 2022, which application is incorporated herein by reference in its entirety for all purposes.

### BACKGROUND

With a total of over 1.6 million new cases each year in the United States as of 2017, cancer represents a prominent worldwide public health problem. See, Siegel et al., 2017, “Cancer statistics,” CA Cancer J Clin. 67(1):7-30. Screening programs and early diagnosis have an important impact in improving disease-free survival and reducing mortality in cancer patients. As noninvasive approaches for early diagnosis foster patient compliance, they can be included in screening programs.

Cell-free nucleic acids (cfNAs) can be found in serum, plasma, urine, and other body fluids (Chan et al., “Clinical Sciences Reviews Committee of the Association of Clinical Biochemists Cell-free nucleic acids in plasma, serum and urine: a new tool in molecular diagnosis,” Ann Clin Biochem. 2003; 40(Pt 2):122-130) representing a “liquid biopsy,” which is a circulating picture of a specific disease. See, De Mattos-Arruda and Caldas, 2016, “Cell-free circulating tumour DNA as a liquid biopsy in breast cancer,” Mol Oncol. 2016; 10(3):464-474. Similarly, cell-free RNA has been proposed as a possible analyte for cancer detection. See, Tzimagiorgis, et al., “Recovering circulating extracellular or cell-free RNA from bodily fluids,” Cancer Epidemiology 2011; 35(6):580-589. These approaches represent potential non-invasive methods of screening for a variety of diseases, such as cancers.

Nevertheless, cancer remains a frequent cause of death worldwide. Over the last several decades, treatment options have improved, yet survival rates remain low. The success of treatment by surgical resection and drug-based approaches is strongly dependent on identification of early-stage tumors. However, current technologies, such as imaging and biomarker-based approaches, frequently cannot identify tumors until the more advanced stages of the disease have set in.

### SUMMARY OF THE INVENTION

In view of the foregoing, there remains a need for non-invasive detection modalities that can identify disease at the earliest stages, when therapeutic interventions have a greater chance of success. Aspects of the present disclosure address this need, and provide other advantages as well.

In some aspects, the present disclosure provides methods of detecting cancer in a subject. In embodiments, the methods comprise: (a) measuring a plurality of target molecules in a biological fluid of the subject, wherein the plurality of target molecules are selected from polypeptides of Table 11; and/or (b) detecting the cancer, wherein detecting the cancer includes detecting one or more of the target molecules above a threshold level. In embodiments, the plurality of target molecules are selected from polypeptides of one or more of Tables 8 or 12-19 (e.g., at least 5, 10, 15, or 20 polypeptides of Tables 8, 11-14 or 17-19).

In some embodiments, (a) the plurality of target molecules further comprises cell-free polynucleotides including (i) cell-free DNA (cfDNA) from genes encoding the polypeptides, and/or (ii) cell-free RNA (cfRNA) transcripts of the genes encoding the polypeptides; and (b) detecting one or more of the target molecules above a threshold level includes (i) detecting one or more of the polypeptides above a first threshold level, and (ii) for each of the polypeptides detected above the first threshold level, detecting a corresponding cell-free polynucleotide above a second threshold level.

In some aspects, the present disclosure provides computer systems for implementing one or more steps in methods of any of the various aspects disclosed herein.

In some aspects, the present disclosure provides non-transitory computer-readable media, having stored thereon computer-readable instructions for implementing one or more steps in methods of any of the various aspects disclosed herein.

### BRIEF DESCRIPTION OF THE DRAWINGS

FIG. 1 is flowchart of a method for preparing a nucleic acid sample for sequencing according to one embodiment.

FIG. 2 is a flow diagram illustrating a method for identifying one or more RNA sequences indicative of a disease state, in accordance with one embodiment of the present invention.

FIG. 3 is a flow diagram illustrating a method for identifying one or more tumor-derived RNA sequences, in accordance with one embodiment of the present invention.

FIG. 4 is a flow diagram illustrating a method for detecting the presence of cancer, determining a state of cancer, monitoring cancer progression, and/or determining cancer type in a subject, in accordance with one embodiment of the present invention.

FIG. 5 is a flow diagram illustrating a method for detecting a disease state from one or more sequence reads derived from one or more targeted RNA molecules, in accordance with one embodiment of the present invention.

FIG. 6 is a flow diagram illustrating a method for detecting the presence of cancer in a subject based on a cancer indicator score, in accordance with one embodiment of the present invention.

FIG. 7 illustrates example results for sensitivity and specificity of sample classification schemes, in accordance with an embodiment.

FIGS. 8A-8C illustrate example results for sensitivity and specificity of sample classification schemes, in accordance with an embodiment.

FIG. 9 depicts the expression levels of 20 dark channel genes in lung cancer with the highest expression level ratio between cancerous and non-cancerous samples. Reads per million (RPM) are plotted as a function of dark channel genes. In each plot, the columns of dots from left to right correspond to groups indicated in the top legend from left to right, respectively (class, anorectal, breast, colorectal, lung, and non-cancer).

FIG. 10 is a ROC curve of the decision tree classifier using a tissue score aggregated from dark channel genes.

FIG. 11 is a flowchart illustrating a method in accordance with some embodiments.

FIG. 12A is a scatter plot of an example PCA (principal component analysis) of stage III TCGA (The Cancer Genome Atlas) FFPE (formalin-fixed paraffin embedded) tissue RNA-seq data. Gene expression levels are plotted in read per million.

FIG. 12B is scatter plot showing example results of CCGA (Circulating Cell-free Genome Atlas) tumor tissue RNA-seq data, projected on TCGA PCA axes. Gene expression levels are plotted in read per million.

FIG. 12C is a scatter plot showing example results of CCGA cancer cell-free RNA (cfRNA) RNA-seq data projected on TCGA PCA axes. Gene expression levels are plotted in read per million.

FIG. 13 is a heatmap of example dark channel biomarker genes. Each column depicts one cfRNA sample, and each row depicts one gene. The color of the rows encodes tissue-specificity (from top to bottom, the tissues are, respectively: breast, lung, and non-specific). The color of the columns encodes the sample groups (from left to right, the cancer types are, respectively: anorectal, breast, colorectal, lung, and non-cancer).

FIG. 14A shows box plots depicting cfRNA expression levels and tissue expression levels of two example breast dark channel biomarkers (DCB) genes (FABP7 and SCGB2A2) in different samples: HER2+, HR+/HER2−, triple negative breast cancer (TNBC), or non-cancer samples.

FIG. 14B shows box plots depicting cfRNA expression levels and tissue expression levels of four example lung DCB genes (SLC34A2, ROS1, SFTPA2, and CXCL17) in different samples: adenocarcinoma, small cell lung cancer, squamous cell carcinoma, or non-cancer samples.

FIG. 15A shows forest plots depicting the detectability of two breast DCB genes (FABP7 and SCGB2A2) for breast cancer samples with matched tumor tissue. The samples IDs are plotted based on their relative tumor fraction in cell-free DNA (cfDNA) (95% CI). FABP7 was detected in samples 4653, 4088, 2037, 3116, and 1202. SCGB2A2 was detected in samples 1656, 2419, 3911, 2367, 2037, 1039, 2139, and 3162. Tumor fraction in cfDNA was measured from SNV allele fractions from the cfDNA enrichment assay.

FIG. 15B shows forest plots depicting the detectability of two breast DCB genes (FABP7 and SCGB2A2) for breast cancer samples with matched tumor tissue. Sample IDs are plotted as a function of tumor content (tumor fraction * tumor tissue expression). FABP7 was detected in samples 4088, 1202, 3116, and 2037. SCGB2A2 was detected in samples 1656, 2419, 2367, 3911, 1039, 2139, 3162, and 2037. Tumor fraction in cfDNA was measured from SNV allele fractions from the cfDNA enrichment assay. Tissue expression was measured from RNA-seq data of matched tumor tissue.

FIGS. 16A-16D illustrate example sequencing results for DCB gene expression in cfRNA and matched tissue for the indicated genes for subjects with breast cancer, lung cancer, or no cancer (normal). The number of read counts is represented on the y-axis.

FIGS. 17A-17B illustrate example classifier workflows.

FIGS. 18A-C illustrate ROC plots showing sensitivity and specificity of example classification schemes.

FIG. 19 illustrates a sample processing and parameter determination method, in accordance with one embodiment of the present invention.

FIGS. 20A-20B illustrate the distributions of select breast- and lung-specific biomarkers in accordance with an embodiment, showing increased signal in breast and lung cancer-derived (respectively) cfRNA versus non-cancer derived cfRNA. Whole transcriptome samples were prepared from the cfRNA of breast cancer, lung cancer, and non-cancer CCGA participants.

FIG. 21 illustrates matched plasma and tissue gene expression from whole transcriptome CCGA breast cancer samples. Results show that high expression in tissue may not necessarily yield high shedding rate into plasma.

FIG. 22 shows a scatter plot illustrating that dark channel expression in CCGA plasma is correlated with CCGA tumor tissue expression for breast cancers. Genes which have mean plasma or tissue expression of zero are transformed here to 1e-4 for visualization purposes.

FIG. 23 is a scatter plot illustrating that dark channel expression in CCGA plasma is correlated with CCGA tumor tissue expression for lung cancers. Genes which have mean plasma or tissue expression of zero are transformed here to 1e-4 for visualization purposes.

FIG. 24 is a graph showing tumor-specific markers in CCGA plasma samples. The plasma log odds ratio was computed for each gene based on observations from all cancer plasma to all non-cancer plasma. The genes shown indicate example dark channel biomarkers.

FIG. 25 is a Venn diagram showing the distribution of cfRNA biomarkers of Table 15 grouped by source and identification method. The 38 biomarkers present in all groupings in the diagram are provided in Table 14. Genes are filtered to optimize for binary detection and to optimize for tissue-of-origin (TOO). The genes filtered for the optimization for binary detection were observed in a CCGA plasma with a log odds ratio >0.1, and the genes with high TCGA expression (>5 RPM) in breast and lung cancers. The genes filtered for optimization for TOO were the genes selected by multiclass random forest method from TCGA tissue, and the genes annotated as breast/lung tumor or tissue specific in Human Protein Atlas.

FIGS. 26A-26D illustrate levels of selected biomarkers detected in breast and/or lung cancer, as compared to non-cancer subjects, in accordance with an embodiment. Results show increased signal in breast and/or lung cancer-derived (respectively) cfRNA versus non-cancer derived cfRNA. Whole transcriptome samples were prepared from the cfRNA of breast cancer, lung cancer, and non-cancer CCGA participants.

FIGS. 27A-27C illustrate levels of selected polypeptide biomarkers detected in plasma of breast cancer subjects, as compared to non-cancer subjects. Results show the normalized counts of proteins in breast cancer-derived versus non-cancer-derived plasma samples. The levels of polypeptide were detected were determined using a proximity extension assay (PEA). FIG. 27A shows the level of a polypeptide biomarker in breast cancer-derived versus non-cancer-derived plasma samples. FIG. 27B shows the levels of selected polypeptide biomarkers across different cohorts. FIG. 27C shows the level of a polypeptide biomarker in breast cancer-derived, lung-cancer-derived, and non-cancer derived plasma samples.

FIGS. 28A-28C illustrate levels of selected polypeptide biomarkers detected in plasma of lung cancer subjects, as compared to non-cancer subjects. Results show the normalized counts of proteins in lung cancer-derived versus non-cancer-derived plasma samples. FIG. 28A shows the level of a polypeptide biomarker in lung cancer-derived versus non-cancer-derived plasma samples. FIG. 28B shows the levels of polypeptide biomarkers identified as drivers of performance in distinguishing low-signal lung cancer-derived plasma samples from non-cancer-derived plasma samples. FIG. 28C shows the levels of selected polypeptide biomarkers across different cohorts.

### DETAILED DESCRIPTION

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit, unless the context clearly dictates otherwise, between the upper and lower limit of that range and any other stated or intervening value in that stated range, as well as each of the provided endpoints of the range, is encompassed within the invention. The upper and lower limits of these smaller ranges may independently be included in the smaller ranges encompassed within the invention, subject to any specifically excluded limit in the stated range.

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), provides one skilled in the art with a general guide to many of the terms used in the present application, as do the following, each of which is incorporated by reference herein in its entirety: Komberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Abbas et al, Cellular and Molecular Immunology, 6th edition (Saunders, 2007).

All publications mentioned herein are expressly incorporated herein by reference to disclose and describe the methods and/or materials in connection with which the publications are cited.

The terms “polynucleotide”, “nucleic acid” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

In general, the term “target polynucleotide” refers to a nucleic acid molecule or polynucleotide in a starting population of nucleic acid molecules having a target sequence whose presence, amount, and/or nucleotide sequence, or changes in one or more of these, are desired to be determined. In general, the term “target sequence” refers to a nucleic acid sequence on a single strand of nucleic acid. The target sequence may be a portion of a gene, a regulatory sequence, genomic DNA, cDNA, RNA including mRNA, miRNA, rRNA, or others. The target sequence may be a target sequence from a sample or a secondary target such as a product of an amplification reaction. A polypeptide encoded by a target polynucleotide, or a portion thereof, is referred to herein as a “target polypeptide.” Target polynucleotides and target polypeptides are encompassed by the term “target molecule.”

The terms “marker” and “biomarker” are used interchangeably herein to refer to a target polynucleotide (e.g., a gene or an identifiable sequence fragment thereof), or a polypeptide encoded thereby, the presence, level or concentration of which is associated with a particular biological state (e.g., a disease state, such as presence of cancer in general, or a particular cancer type and/or stage). In embodiments, a marker is a polypeptide encoded by a particular gene, or a portion thereof. In embodiments, a marker is a cfRNA of a particular gene, changes in the level of which may be detected by sequencing. cfRNA biomarkers may be referred to herein with reference to the gene from which the cfRNA derives, but does not necessitate detection of the entire gene transcript. In embodiments, only fragments of a particular gene transcript are detected. In embodiments, detecting the presence and/or level of a particular gene comprises detecting one or more cfRNA fragments comprising different sequence fragments (overlapping or non-overlapping) derived from transcripts of the same gene, which may be scored collectively as part of the same “biomarker.” Additional information relating to recited gene designations, including sequence information (e.g., DNA, RNA, and amino acid sequences), full names of genes commonly identified by way of gene symbol, and the like are available in publicly accessible databases known to those skilled in the art, such as databases available from the National Center for Biotechnology Information (www.ncbi.nlm.nih.gov/), including GenBank (www.ncbi.nlm.nih.gov/genbank/) and the NCBI Protein database (www.ncbi.nlm.nih.gov/protein/), and UniProt (www.uniprot.org).

The term “amplicon” as used herein means the product of a polynucleotide amplification reaction; that is, a clonal population of polynucleotides, which may be single stranded or double stranded, which are replicated from one or more starting sequences. The one or more starting sequences may be one or more copies of the same sequence, or they may be a mixture of different sequences. Preferably, amplicons are formed by the amplification of a single starting sequence. Amplicons may be produced by a variety of amplification reactions whose products comprise replicates of the one or more starting, or target, nucleic acids. In one aspect, amplification reactions producing amplicons are “template-driven” in that base pairing of reactants, either nucleotides or oligonucleotides, have complements in a template polynucleotide that are required for the creation of reaction products. In one aspect, template-driven reactions are primer extensions with a nucleic acid polymerase, or oligonucleotide ligations with a nucleic acid ligase. Such reactions include, but are not limited to, polymerase chain reactions (PCRs), linear polymerase reactions, nucleic acid sequence-based amplification (NASBAs), rolling circle amplifications, and the like, disclosed in the following references, each of which are incorporated herein by reference herein in their entirety: Mullis et al, U.S. Pat. Nos. 4,683,195; 4,965,188; 4,683,202; 4,800,159 (PCR); Gelfand et al, U.S. Pat. No. 5,210,015 (real-time PCR with “taqman” probes); Wittwer et al, U.S. Pat. No. 6,174,670; Kacian et al, U.S. Pat. No. 5,399,491 (“NASBA”); Lizardi, U.S. Pat. No. 5,854,033; Aono et al, Japanese patent publ. JP 4-262799 (rolling circle amplification); and the like. In one aspect, amplicons of the invention are produced by PCRs. An amplification reaction may be a “real-time” amplification if a detection chemistry is available that permits a reaction product to be measured as the amplification reaction progresses, e.g., “real-time PCR”, or “real-time NASBA” as described in Leone et al, Nucleic Acids Research, 26: 2150-2155 (1998), and like references.

The term “amplifying” means performing an amplification reaction. A “reaction mixture” means a solution containing all the necessary reactants for performing a reaction, which may include, but is not be limited to, buffering agents to maintain pH at a selected level during a reaction, salts, co-factors, scavengers, and the like.

The terms “fragment” or “segment”, as used interchangeably herein, refer to a portion of a larger molecule. A polynucleotide, for example, can be broken up, or fragmented into, a plurality of segments, either through natural processes, as is the case with, e.g., cfDNA fragments that can naturally occur within a biological sample, or through in vitro manipulation. Various methods of fragmenting nucleic acid are well known in the art. These methods may be, for example, either chemical or physical or enzymatic in nature. Enzymatic fragmentation may include partial degradation with a DNase; partial depurination with acid; the use of restriction enzymes; intron-encoded endonucleases; DNA-based cleavage methods, such as triplex and hybrid formation methods, that rely on the specific hybridization of a nucleic acid segment to localize a cleavage agent to a specific location in the nucleic acid molecule; or other enzymes or compounds which cleave a polynucleotide at known or unknown locations. Physical fragmentation methods may involve subjecting a polynucleotide to a high shear rate. High shear rates may be produced, for example, by moving DNA through a chamber or channel with pits or spikes, or forcing a DNA sample through a restricted size flow passage, e.g., an aperture having a cross sectional dimension in the micron or submicron range. Other physical methods include sonication and nebulization. Combinations of physical and chemical fragmentation methods may likewise be employed, such as fragmentation by heat and ion-mediated hydrolysis. See, e.g., Sambrook et al., “Molecular Cloning: A Laboratory Manual,” 3rd Ed. Cold Spring Harbor Laboratory Press, Cold Spring Harbor, N. Y. (2001) (“Sambrook et al.) which is incorporated herein by reference for all purposes. These methods can be optimized to digest a nucleic acid into fragments of a selected size range.

The terms “polymerase chain reaction” or “PCR”, as used interchangeably herein, mean a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors that are well-known to those of ordinary skill in the art, e.g., exemplified by the following references: McPherson et al, editors, PCR: A Practical Approach and PCR2: A Practical Approach (IRL Press, Oxford, 1991 and 1995, respectively). For example, in a conventional PCR using Taq DNA polymerase, a double stranded target nucleic acid may be denatured at a temperature >90° C., primers annealed at a temperature in the range 50-75° C., and primers extended at a temperature in the range 72-78° C. The term “PCR” encompasses derivative forms of the reaction, including, but not limited to, RT-PCR, real-time PCR, nested PCR, quantitative PCR, multiplexed PCR, and the like. The particular format of PCR being employed is discernible by one skilled in the art from the context of an application. Reaction volumes can range from a few hundred nanoliters, e.g., 200 nL, to a few hundred μL, e.g., 200 μL. “Reverse transcription PCR,” or “RT-PCR,” means a PCR that is preceded by a reverse transcription reaction that converts a target RNA to a complementary single stranded DNA, which is then amplified, an example of which is described in Tecott et al, U.S. Pat. No. 5,168,038, the disclosure of which is incorporated herein by reference in its entirety. “Real-time PCR” means a PCR for which the amount of reaction product, i.e., amplicon, is monitored as the reaction proceeds. There are many forms of real-time PCR that differ mainly in the detection chemistries used for monitoring the reaction product, e.g., Gelfand et al, U.S. Pat. No. 5,210,015 (“tagman”); Wittwer et al, U.S. Pat. Nos. 6,174,670 and 6,569,627 (intercalating dyes); Tyagi et al, U.S. Pat. No. 5,925,517 (molecular beacons); the disclosures of which are hereby incorporated by reference herein in their entireties. Detection chemistries for real-time PCR are reviewed in Mackay et al, Nucleic Acids Research, 30: 1292-1305 (2002), which is also incorporated herein by reference. “Nested PCR” means a two-stage PCR wherein the amplicon of a first PCR becomes the sample for a second PCR using a new set of primers, at least one of which binds to an interior location of the first amplicon. As used herein, “initial primers” in reference to a nested amplification reaction mean the primers used to generate a first amplicon, and “secondary primers” mean the one or more primers used to generate a second, or nested, amplicon. “Asymmetric PCR” means a PCR wherein one of the two primers employed is in great excess concentration so that the reaction is primarily a linear amplification in which one of the two strands of a target nucleic acid is preferentially copied. The excess concentration of asymmetric PCR primers may be expressed as a concentration ratio. Typical ratios are in the range of from 10 to 100. “Multiplexed PCR” means a PCR wherein multiple target sequences (or a single target sequence and one or more reference sequences) are simultaneously carried out in the same reaction mixture, e.g., Bernard et al, Anal. Biochem., 273: 221-228 (1999)(two-color real-time PCR). Usually, distinct sets of primers are employed for each sequence being amplified. Typically, the number of target sequences in a multiplex PCR is in the range of from 2 to 50, or from 2 to 40, or from 2 to 30. “Quantitative PCR” means a PCR designed to measure the abundance of one or more specific target sequences in a sample or specimen. Quantitative PCR includes both absolute quantitation and relative quantitation of such target sequences. Quantitative measurements are made using one or more reference sequences or internal standards that may be assayed separately or together with a target sequence. The reference sequence may be endogenous or exogenous to a sample or specimen, and in the latter case, may comprise one or more competitor templates. Typical endogenous reference sequences include segments of transcripts of the following genes: β-actin, GAPDH, β₂-microglobulin, ribosomal RNA, and the like. Techniques for quantitative PCR are well-known to those of ordinary skill in the art, as exemplified in the following references, which are incorporated by reference herein in their entireties: Freeman et al, Biotechniques, 26: 112-126 (1999); Becker-Andre et al, Nucleic Acids Research, 17: 9437-9447 (1989); Zimmerman et al, Biotechniques, 21: 268-279 (1996); Diviacco et al, Gene, 122: 3013-3020 (1992); and Becker-Andre et al, Nucleic Acids Research, 17: 9437-9446 (1989).

The term “primer” as used herein means an oligonucleotide, either natural or synthetic, that is capable, upon forming a duplex with a polynucleotide template, of acting as a point of initiation of nucleic acid synthesis and being extended from its 3′ end along the template so that an extended duplex is formed. Extension of a primer is usually carried out with a nucleic acid polymerase, such as a DNA or RNA polymerase. The sequence of nucleotides added in the extension process is determined by the sequence of the template polynucleotide. Usually, primers are extended by a DNA polymerase. Primers usually have a length in the range of from 14 to 40 nucleotides, or in the range of from 18 to 36 nucleotides. Primers are employed in a variety of nucleic amplification reactions, for example, linear amplification reactions using a single primer, or polymerase chain reactions, employing two or more primers. Guidance for selecting the lengths and sequences of primers for particular applications is well known to those of ordinary skill in the art, as evidenced by the following reference that is incorporated by reference herein in its entirety: Dieffenbach, editor, PCR Primer: A Laboratory Manual, 2nd Edition (Cold Spring Harbor Press, New York, 2003).

The terms “polypeptide”, “peptide” and “protein” are used interchangeably herein to refer to polymers of amino acids of any length. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics. In embodiments, a polypeptide is encoded by a target polynucleotide, or a portion thereof.

The terms “subject” and “patient” are used interchangeably herein and refer to a human or non-human animal who is known to have, or potentially has, a medical condition or disorder, such as, e.g., a cancer.

The term “sequence read” as used herein refers to a string of nucleotides from part of, or all of, a nucleic acid molecule from a sample obtained from a subject. A sequence read may be a short string of nucleotides (e.g., 20-150) sequenced from a nucleic acid fragment, a short string of nucleotides at one or both ends of a nucleic acid fragment, or the sequencing of the entire nucleic acid fragment that exists in the biological sample. Sequence reads can be obtained through various methods known in the art. For example, a sequence read may be obtained in a variety of ways, e.g., using sequencing techniques or using probes. e.g., in hybridization arrays or capture probes, or amplification techniques, such as the polymerase chain reaction (PCR) or linear amplification using a single primer or isothermal amplification.

The term “read segment” or “read” as used herein refers to any nucleotide sequences, including sequence reads obtained from a subject and/or nucleotide sequences, derived from an initial sequence read from a sample. For example, a read segment can refer to an aligned sequence read, a collapsed sequence read, or a stitched read. Furthermore, a read segment can refer to an individual nucleotide base, such as a single nucleotide variant.

The term “enrich” as used herein means to increase a proportion of one or more target nucleic acids in a sample. An “enriched” sample or sequencing library is therefore a sample or sequencing library in which a proportion of one of more target nucleic acids has been increased with respect to non-target nucleic acids in the sample.

In general, the terms “cell-free,” “circulating,” and “extracellular” as applied to polynucleotides (e.g. “cell-free RNA” and “cell-free DNA”) are used interchangeably to refer to polynucleotides present in a sample from a subject or portion thereof that can be isolated or otherwise manipulated without applying a lysis step to the sample as originally collected (e.g., as in lysis for the extraction from cells or viruses). Cell-free polynucleotides are thus unencapsulated or “free” from the cells or viruses from which they originate, even before a sample of the subject is collected. Cell-free polynucleotides may be produced as a byproduct of cell death (e.g. apoptosis or necrosis) or cell shedding, releasing polynucleotides into surrounding body fluids or into circulation. Accordingly, cell-free polynucleotides may be isolated from a non-cellular fraction of blood (e.g. serum or plasma), from other bodily fluids (e.g. urine), or from non-cellular fractions of other types of samples. The term “cell-free RNA” or “cfRNA” refers to ribonucleic acid fragments that circulate in a subject's body (e.g., bloodstream) and may originate from one or more healthy cells and/or from one or more cancer cells. Likewise, “cell-free DNA” or “cfDNA” refers to deoxyribonucleic acid molecules that circulate in a subject's body (e.g., bloodstream) and may originate from one or more healthy cells and/or from one or more cancer cells.

The term “circulating tumor RNA” or “ctRNA” refers to ribonucleic acid fragments that originate from tumor cells or other types of cancer cells, which may be released into a subject's body (e.g., bloodstream) as a result of biological processes, such as apoptosis or necrosis of dying cells, or may be actively released by viable tumor cells.

The term “dark channel RNA” or “dark channel cfRNA molecule” or “dark channel gene” as used herein refers to an RNA molecule or gene whose expression in healthy cells is very low or nonexistent. Accordingly, identification, detection, and/or quantification of dark channel RNA (cfRNA) molecules improves signal-to-noise, and improvements in sensitivity and specificity, in assessment of a disease state, such as cancer.

“Treating” or “treatment” as used herein includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Treating” and “treatment” as used herein includes prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is no prophylactic treatment.

The term “prevent”, as pertains to a disease or condition of a subject, refers to a decrease in the occurrence of one or more corresponding symptoms in the subject. As indicated above, the prevention may be complete (no detectable symptoms) or partial, such that fewer symptoms are observed, and/or with lower incidence, than would likely occur absent treatment.

“Anti-cancer agent” and “anticancer agent” are used in accordance with their plain ordinary meaning and refers to a composition (e.g. compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer. Examples of anti-cancer agents include, but are not limited to, MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethylmelamine, thiotepa), alkyl sulfonates (e.g., busulfan), nitrosoureas (e.g., carmustine, lomustine, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxuridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaliplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossypol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; elformithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin Il (including recombinant interleukin II, or rlL.sub.2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. Taxol™ (i.e. paclitaxel), Taxotere™, compounds comprising the taxane skeleton, Erbulozole (i.e. R-55104), Dolastatin 10 (i.e. DLS-10 and NSC-376128), Mivobulin isethionate (i.e. as CI-980), Vincristine, NSC-639829, Discodermolide (i.e. as NVP-XX-A-296), ABT-751 (Abbott, i.e. E-7010), Altohyrtins (e.g. Altohyrtin A and Altohyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-310705), 21-hydroxyepothilone D (i.e. Desoxyepothilone F and dEpoF), 26-fluoroepothilone, Auristatin PE (i.e. NSC-654663), Soblidotin (i.e. TZT-1027), LS-4559-P (Pharmacia, i.e. LS-4577), LS-4578 (Pharmacia, i.e. LS-477-P), LS-4477 (Pharmacia), LS-4559 (Pharmacia), RPR-112378 (Aventis), Vincristine sulfate, DZ-3358 (Daiichi), FR-182877 (Fujisawa, i.e. WS-9885B), GS-164 (Takeda), GS-198 (Takeda), KAR-2 (Hungarian Academy of Sciences), BSF-223651 (BASF, i.e. ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e. LY-355703), AC-7739 (Ajinomoto, i.e. AVE-8063A and CS-39.HCl), AC-7700 (Ajinomoto, i.e. AVE-8062, AVE-8062A, CS-39-L-Ser.HCl, and RPR-258062A), Vitilevuamide, Tubulysin A, Canadensol, Centaureidin (i.e. NSC-106969), T-138067 (Tularik, i.e. T-67, TL-138067 and TI-138067), COBRA-1 (Parker Hughes Institute, i.e. DDE-261 and WHI-261), H10 (Kansas State University), H16 (Kansas State University), Oncocidin A1 (i.e. BTO-956 and DIME), DDE-313 (Parker Hughes Institute), Fijianolide B, Laulimalide, SPA-2 (Parker Hughes Institute), SPA-1 (Parker Hughes Institute, i.e. SPIKET-P), 3-IAABU (Cytoskeleton/Mt. Sinai School of Medicine, i.e. MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e. MF-191), TMPN (Arizona State University), Vanadocene acetylacetonate, T-138026 (Tularik), Monsatrol, lanocide (i.e. NSC-698666), 3-IAABE (Cytoskeleton/Mt. Sinai School of Medicine), A-204197 (Abbott), T-607 (Tuiarik, i.e. T-900607), RPR-115781 (Aventis), Eleutherobins (such as Desmethyleleutherobin, Desaetyleleutherobin, lsoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (i.e. NSCL-96F037), D-68838 (Asta Medica), D-68836 (Asta Medica), Myoseverin B, D-43411 (Zentaris, i.e. D-81862), A-289099 (Abbott), A-318315 (Abbott), HTI-286 (i.e. SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethylstilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guérin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to 111In, 90Y, or 1311, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™), panitumumab (Vectibix™) vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like.

An “epigenetic inhibitor” as used herein, refers to an inhibitor of an epigenetic process, such as DNA methylation (a DNA methylation Inhibitor) or modification of histones (a Histone Modification Inhibitor). An epigenetic inhibitor may be a histone-deacetylase (HDAC) inhibitor, a DNA methyltransferase (DNMT) inhibitor, a histone methyltransferase (HMT) inhibitor, a histone demethylase (HDM) inhibitor, or a histone acetyltransferase (HAT). Examples of HDAC inhibitors include Vorinostat, romidepsin, CI-994, Belinostat, Panobinostat, Givinostat, Entinostat, Mocetinostat, SRT501, CUDC-101, JNJ-26481585, or PC124781. Examples of DNMT inhibitors include azacitidine and decitabine. Examples of HMT inhibitors include EPZ-5676. Examples of HDM inhibitors include pargyline and tranylcypromine. Examples of HAT inhibitors include CCT077791 and garcinol.

A “multi-kinase inhibitor” is a small molecule inhibitor of at least one protein kinase, including tyrosine protein kinases and serine/threonine kinases. A multi-kinase inhibitor may include a single kinase inhibitor. Multi-kinase inhibitors may block phosphorylation. Multi-kinases inhibitors may act as covalent modifiers of protein kinases. Multi-kinase inhibitors may bind to the kinase active site or to a secondary or tertiary site inhibiting protein kinase activity. A multi-kinase inhibitor may be an anti-cancer multi-kinase inhibitor. Exemplary anti-cancer multi-kinase inhibitors include dasatinib, sunitinib, erlotinib, bevacizumab, vatalanib, vemurafenib, vandetanib, cabozantinib, ponatinib, axitinib, ruxolitinib, regorafenib, crizotinib, bosutinib, cetuximab, gefitinib, imatinib, lapatinib, lenvatinib, mubritinib, nilotinib, panitumumab, pazopanib, trastuzumab, or sorafenib.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

Aspects of the disclosed subject matter includes methods for detecting a disease state, (e.g., a presence or absence of cancer), and/or a tissue of origin of the disease in a subject, based on analysis of one or more target molecules in a sample from the subject. In some embodiments, a method for detecting a disease state in a subject comprises isolating a biological test sample from the subject, wherein the biological test sample comprises a plurality of polypeptides, and performing a detection assay to determine the presence or amount of one or more target polypeptides in the plurality. Information concerning the presence or amount of the one or more target polypeptides may be combined with the presence or amount of one or more target polynucleotides encoding the one or more target polypeptides, or a fragment thereof. In some embodiments, a method for detecting a disease state in a subject comprises isolating a biological test sample from the subject, wherein the biological test sample comprises a plurality of cell-free ribonucleic acid (cfRNA) molecules, extracting the cfRNA molecules from the biological test sample, performing a sequencing procedure on the extracted cfRNA molecules to generate a plurality of sequence reads, performing a filtering procedure to generate an excluded population of sequence reads that originate from one or more healthy cells, and a non-excluded population of sequence reads, and performing a quantification procedure on the non-excluded sequence reads. In embodiments, the methods comprise detecting the disease state in the subject when the quantification procedure produces a value that exceeds a threshold. In embodiments, detecting one or more non-excluded sequence reads above a threshold comprises (i) detection, (ii) detection above background, and/or (iii) detection at a level that is greater than a level of corresponding sequence reads in subjects that do not have the condition. In various embodiments, the threshold value is an integer that ranges from about or exactly 1 to about or exactly 10, such as about or exactly 2, 3, 4, 5, 6, 7, 8, or about or exactly 9. In some embodiments, the threshold is a non-integer value, ranging from about or exactly 0.1 to about or exactly 0.9, such as about or exactly 0.2, 0.3, 0.4, 0.5, 0.6, 0.7 or about or exactly 0.8. In embodiments, target polypeptides and target polynucleotides are from the same sample or from different samples collected at about the same time.

In some embodiments, the methods involve the use of sequencing a procedure for detecting and quantifying the cfRNA molecules that are extracted from a biological test sample. For example, in various embodiments a sequencing procedure involves performing a reverse transcription procedure on the cfRNA molecules to produce a plurality of cDNA/RNA hybrid molecules, degrading the RNA of the hybrid molecules to produce a plurality of single-stranded cDNA molecule templates, synthesizing a plurality of double-stranded DNA molecules from the single-stranded cDNA molecule templates, ligating a plurality of double-stranded DNA adapters to the plurality of double-stranded DNA molecules producing a sequencing library, and performing a sequencing procedure on at least a portion of the sequencing library to obtain a plurality of sequence reads. In various embodiments, synthesizing the double-stranded DNA molecules involves performing a strand-displacement reverse transcriptase procedure.

In some embodiments, the methods utilize whole transcriptome sequencing procedures. In other embodiments, a sequencing procedure involves a targeted sequencing procedure, wherein one or more of the cfRNA molecules are enriched from the biological test sample before preparing a sequencing library. In accordance with this embodiment, one or more cfRNA molecules indicative of the disease state are targeted for enrichment. For example, in some embodiments, the one or more targeted cfRNA molecules are derived from one or more genes selected from the group consisting of: AGR2, BPIFA1, CASP14, CSN1S1, DISP2, EIF2D, FABP7, GABRG1, GNAT3, GRHL2, HOXC10, ID12-AS1, KRT16P2, LALBA, LINC00163, NKX2-1, OPN1SW, PADI3, PTPRZ1, ROS1, S100A7, SCGB2A2, SERPINB5, SFTA3, SFTPA2, SLC34A2, TFF1, VTCN1, WFDC2, MUC5B, SMIM22, CXCL17, RNU1-1, and KLK5, and can comprise any combination thereof. In some embodiments, one or more target RNA molecules are derived from one or more genes selected from the group consisting of ROS1, NKX2-1, GGTLC1, SLC34A2, SFTPA2, BPIFA1, SFTA3, GABRG1, AGR2, GNAT3, MUC5B, SMIM22, CXCL17, and WFDC2, and can comprise any combination thereof. In some embodiments, one or more target RNA molecules are derived from one or more genes selected from the group consisting of SCGB2A2, CSN1S1, VTCN, FABP7, LALBA, RNU1-1, OPN1SW, CASP14, KLK5, and WFDC2, and can comprise any combination thereof. In some embodiments, one or more target RNA molecules are derived from one or more genes selected from the group consisting of CASP14, CRABP2, FABP7, SCGB2A2, SERPINB5, TRGV10, VGLL1, TFF1, and AC007563.5, and can comprise any combination thereof. In still other embodiments, the targeted RNA molecule is derived from the AKR1B10, C3, and/or PIEXO2 gene(s).

Aspects of the disclosed subject matter involve analysis of one or more dark channel RNA molecules, and/or polypeptides encoded thereby, whose expression in the plasma of healthy subjects is very low or nonexistent. Due to their low expression level in the plasma of healthy subjects, dark channel RNA molecules provide a high signal to noise ratio that can be used in conjunction with the present methods.

Some aspects of the disclosed subject matter involve filtering procedures that are used to generate an excluded population of sequence reads that originate from one or more healthy cells, and a non-excluded population of sequence reads that are used in subsequent analyses. In various embodiments, the filtering procedure involves comparing each sequence read from the cfRNA molecules extracted from the biological test sample to a control data set of RNA sequences, identifying one or more sequence reads that match one or more sequence reads in the control data set of RNA sequences, and placing each sequence read that matches the one or more sequence reads in the control data set of RNA sequences in the excluded population of sequence reads.

In some embodiments, a control data set of target molecules (e.g., DNA or RNA sequences) includes a plurality of sequence reads obtained from one or more healthy subjects. In some embodiments, a control data set of RNA sequences includes a plurality of sequence reads obtained from a plurality of blood cells from the subject. For example, in some embodiments, a plurality of sequence reads are obtained from a subject's white blood cells (WBCs). In embodiments, a control data set of target molecules includes data for the presence or amount of target molecules (e.g., polypeptides and/or polynucleotides) for a reference condition, such as a population known to have or known not to have a particular condition under examination, or a given subject tested at a different time (e.g., before developing a particular condition under examination).

### Biological Samples

In various embodiments, the present disclosure involves obtaining a test sample, e.g., a biological test sample, such as a tissue and/or body fluid sample, from a subject for purposes of analyzing a plurality of target molecules (e.g., a plurality of polypeptides, cfDNA, and/or cfRNA molecules) therein. Samples in accordance with embodiments of the invention can be collected in any clinically-acceptable manner. Any sample suspected of containing a plurality of target molecules can be used in conjunction with the methods of the present invention. In some embodiments, a sample can comprise a tissue, a body fluid, or a combination thereof. In some embodiments, a biological sample is collected from a healthy subject. In some embodiments, a biological sample is collected from a subject who is known to have a particular disease or disorder (e.g., a particular cancer or tumor). In some embodiments, a biological sample is collected from a subject who is suspected of having a particular disease or disorder.

As used herein, the term “tissue” refers to a mass of connected cells and/or extracellular matrix material(s). Non-limiting examples of tissues that are commonly used in conjunction with the present methods include skin, hair, finger nails, endometrial tissue, nasal passage tissue, central nervous system (CNS) tissue, neural tissue, eye tissue, liver tissue, kidney tissue, placental tissue, mammary gland tissue, gastrointestinal tissue, musculoskeletal tissue, genitourinary tissue, bone marrow, and the like, derived from, for example, a human or non-human mammal. Tissue samples in accordance with embodiments of the invention can be prepared and provided in the form of any tissue sample types known in the art, such as, for example and without limitation, formalin-fixed paraffin-embedded (FFPE), fresh, and fresh frozen (FF) tissue samples.

As used herein, the terms “body fluid” and “biological fluid” refer to a liquid material derived from a subject, e.g., a human or non-human mammal. Non-limiting examples of body fluids that are commonly used in conjunction with the present methods include mucous, blood, plasma, serum, serum derivatives, synovial fluid, lymphatic fluid, bile, phlegm, saliva, sweat, tears, sputum, amniotic fluid, menstrual fluid, vaginal fluid, semen, urine, cerebrospinal fluid (CSF), such as lumbar or ventricular CSF, gastric fluid, a liquid sample comprising one or more material(s) derived from a nasal, throat, or buccal swab, a liquid sample comprising one or more materials derived from a lavage procedure, such as a peritoneal, gastric, thoracic, or ductal lavage procedure, and the like.

In some embodiments, a sample can comprise a fine needle aspirate or biopsied tissue. In some embodiments, a sample can comprise media containing cells or biological material. In some embodiments, a sample can comprise a blood clot, for example, a blood clot that has been obtained from whole blood after the serum has been removed. In some embodiments, a sample can comprise stool. In one preferred embodiment, a sample is drawn whole blood. In one aspect, only a portion of a whole blood sample is used, such as plasma, red blood cells, white blood cells, and platelets. In some embodiments, a sample is separated into two or more component parts in conjunction with the present methods. For example, in some embodiments, a whole blood sample is separated into plasma, red blood cell, white blood cell, and platelet components.

In some embodiments, a sample includes a plurality of polypeptides and/or nucleic acids not only from the subject from which the sample was taken, but also from one or more other organisms, such as viral DNA/RNA that is present within the subject at the time of sampling.

Nucleic acid and/or polypeptides can be extracted from a sample according to any suitable methods known in the art, and the extracted nucleic acid can be utilized in conjunction with the methods described herein. See, e.g., Maniatis, et al., Molecular Cloning: A Laboratory Manual, Cold Spring Harbor, N.Y., pp. 280-281, 1982, the contents of which are incorporated by reference herein in their entirety. In one preferred embodiment, polypeptides are purified from a sample. In some embodiments, cell free nucleic acid (e.g., cfRNA and/or cfDNA) is extracted from a sample.

In embodiments, the sample is a “matched” or “paired” sample. In general, the terms “matched sample” and “paired sample” refer to a pair of samples of different types collected from the same subject, preferably at about the same time (e.g., as part of a single procedure or office visit, or on the same day). In embodiments, the different types are a tissue sample (e.g., cancer tissue, as in a resection or biopsy sample) and a biological fluid sample (e.g., blood or a blood fraction). The terms may also be used to refer to polypeptides and/or polynucleotides derived from the matched sample (e.g., polynucleotides extracted from a cancer tissue, paired with cell-free polynucleotides from a matched biological fluid sample), or sequencing reads thereof. In embodiments, a plurality of paired samples are analyzed, such as in identifying cancer biomarkers. The plurality of paired samples may be from the same individual collected at different times (e.g., as in a paired sample from an early stage of cancer, and a paired sample from a later stage of cancer), from different individuals at the same or different times, or a combination of these. In embodiments, the matched samples are from different subjects. In embodiments, the matched samples in a plurality are from subjects with the same cancer type, and optionally the same cancer stage.

### Example Assay Protocol

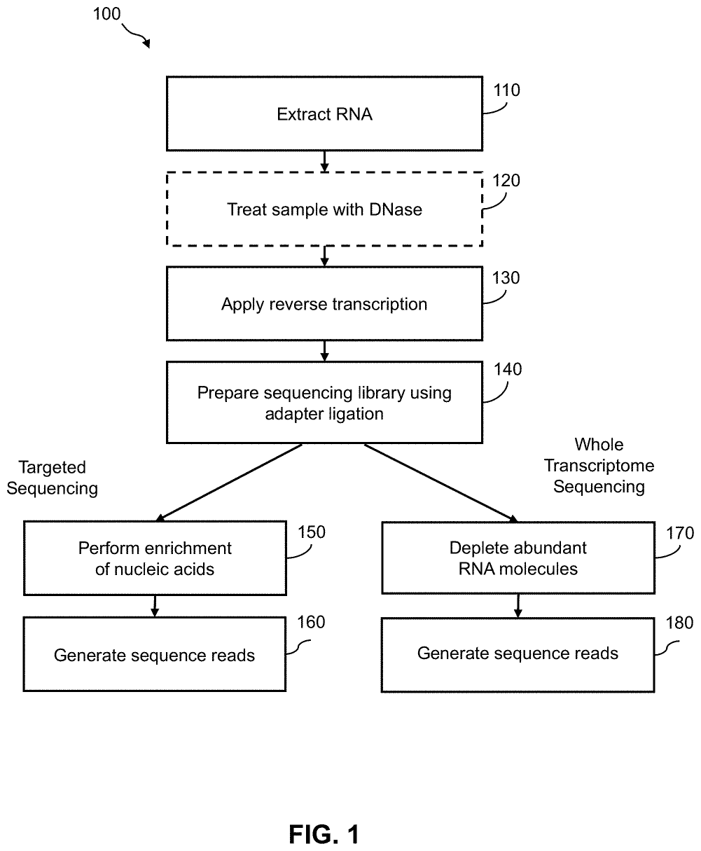

FIG. 1 is flowchart of a method 100 for preparing a nucleic acid sample for sequencing according to one embodiment. The method 100 includes, but is not limited to, the following steps. For example, any step of the method 100 may comprise a quantitation sub-step for quality control or other laboratory assay procedures known to one skilled in the art.

In step 110, a ribonucleic acid (RNA) sample is extracted from a subject. The RNA sample may comprise the whole human transcriptome, or any subset of the human transcriptome. The sample may be extracted from a subject known to have or suspected of having a disease (e.g., cancer). The sample may include blood, plasma, serum, urine, fecal, saliva, other types of bodily fluids, or any combination thereof. In some embodiments, methods for drawing a blood sample (e.g., syringe or finger prick) may be less invasive than procedures for obtaining a tissue biopsy, which may require surgery. The extracted sample may further comprise cfDNA. If a subject has a disease (e.g., cancer), cfRNA in an extracted sample may be present at a detectable level for diagnosis.

In step 120, the nucleic acid sample including RNA molecules is optionally treated with a DNase enzyme. The DNase may remove DNA molecules from the nucleic acid sample to reduce DNA contamination of the RNA molecules. After RNA molecules are converted into DNA, it may be difficult to distinguish the RNA-converted DNA and genomic DNA originally found in the nucleic acid sample. Applying the DNase allows for targeted amplification of molecules originating from cfRNA. The DNase process may include steps for adding a DNase buffer, mixing the sample applied with DNase using a centrifuge, and incubation. In some embodiments, step 120 includes one or more processes based on the DNase treatment protocol described in the Qiagen QIAamp Circulating Nucleic Acid Handbook.

In step 130, a reverse transcriptase enzyme is used to convert the RNA molecules in the nucleic acid sample into complementary DNA (cDNA). The reverse transcriptase process may include a first-strand synthesis step (generation of a cDNA strand via reverse transcription), degradation of the RNA strand to produce a single-stranded cDNA molecule, and synthesis of a double-stranded DNA molecules from the single-stranded cDNA molecule using a polymerase. During first-strand synthesis, a primer anneals to the 3′ end of a RNA molecule. During second-strand synthesis, a different primer anneals to the 3′ end of the cDNA molecule.

In step 140, a sequencing library is prepared. For example, as is well known in the art, adapters can be ligated to one or both ends of a dsDNA molecule to prepare a library for sequencing. In one embodiment, the adapters utilized may include one or more sequencing oligonucleotides for use in subsequent cluster generation and/or sequencing (e.g., known P5 and P7 sequences for used in sequencing by synthesis (SBS) (Illumina, San Diego, Calif.)). In another embodiment, the adapter includes a sample specific index sequence, such that, after library preparation, the library can be combined with one or more other libraries prepared from individual samples, thereby allowing for multiplex sequencing. The sample specific index sequence can comprise a short oligonucleotide sequence having a length of from about or exactly 2 nt to about or exactly 20 nt, from about or exactly 2 nt to about or exactly 10 nt, from about or exactly 2 to about or exactly 8 nt, or from about or exactly 2 to about or exactly 6 nt. In another embodiment, the sample specific index sequence can comprise a short oligonucleotide sequence greater than about or exactly 2, 3, 4, 5, 6, 7, or 8 nucleotides (nt) in length.

Optionally, during library preparation, unique molecular identifiers (UMI) can be added to the nucleic acid molecules in the sample through adapter ligation. The UMIs are short nucleic acid sequences (e.g., 4-10 base pairs) that are added to one or both ends of nucleic acid fragments during adapter ligation. In some embodiments, UMIs are degenerate base pairs that serve as a unique tag that can be used to identify sequence reads originating from a specific nucleic acid fragment. During PCR amplification following adapter ligation, the UMIs are replicated along with the attached nucleic acid fragment, which provides a way to identify sequence reads that came from the same original nucleic acid molecule in downstream analysis.

For embodiments including targeted sequencing of RNA, in step 150, targeted nucleic acid sequences are enriched from the library. During enrichment, hybridization probes (also referred to herein as “probes”) are used to target, and pull down, nucleic acid fragments informative for the presence or absence of a disease (e.g., cancer), disease status (e.g., cancer status), or a disease classification (e.g., cancer type or tissue of origin). For a given workflow, the probes may be designed to anneal (or hybridize) to a target (complementary) nucleic acid strand (e.g., a DNA strand converted from RNA). The probes may range in length from 10s, 100s, or 1000s of base pairs. In one embodiment, the probes are designed based on a gene panel to analyze particular target regions of the genome (e.g., of the human or another organism) that are suspected to correspond to certain cancers or other types of diseases. Moreover, the probes may cover overlapping portions of a target region. In other embodiments, targeted RNA molecules can be enriched using hybridization probes prior to conversion of the RNA molecules to cDNA strands using reverse transcriptase (not shown). In general, any known method in the art can be used to isolate, and enrich for, probe-hybridized target nucleic acids. For example, as is well known in the art, a biotin moiety can be added to the 5′-end of the probes (i.e., biotinylated) to facilitate isolation of target nucleic acids hybridized to probes using a streptavidin-coated surface (e.g., streptavidin-coated beads).

Additionally, for targeted sequencing, in step 160, sequence reads are generated from the enriched nucleic acid sample. Sequencing data may be acquired from the enriched DNA sequences (i.e., DNA sequences derived, or converted, from RNA sequences) by known means in the art. For example, the method 100 may include next generation sequencing (NGS) techniques including synthesis technology (Illumina), pyrosequencing (454 Life Sciences), ion semiconductor technology (Ion Torrent sequencing), single-molecule real-time sequencing (Pacific Biosciences), sequencing by ligation (SOLiD sequencing), nanopore sequencing (Oxford Nanopore Technologies), or paired-end sequencing. In some embodiments, massively parallel sequencing is performed using sequencing-by-synthesis with reversible dye terminators.

In other embodiments, for example, in a whole transcriptome sequencing approach (e.g., instead of targeted sequencing), in step 170, abundant RNA species are depleted from the nucleic acid sample. For example, in some embodiments, ribosomal RNA (rRNA) and/or transfer RNA (tRNA) species can be depleted. Available commercial kits, such as RiboMinus™ (ThermoFisher Scientific) or AnyDeplete (NuGen), can be used for depletion of abundant RNA species. In an embodiment, after depletion of nucleic acids (e.g., converted DNA) derived from abundant RNA molecules, sequence reads are generated in step 180.

In some embodiments, the sequence reads may be aligned to a reference genome using known methods in the art to determine alignment position information. The alignment position information may indicate a beginning position and an end position of a region in the reference genome that corresponds to a beginning nucleotide base and end nucleotide base of a given sequence read. Alignment position information may also include sequence read length, which can be determined from the beginning position and end position. A region in the reference genome may be associated with a gene or a segment of a gene. The reference genome may comprise the whole transcriptome, or any portion thereof (e.g., a plurality of targeted transcripts). In another embodiment, the reference genome can be the whole genome from an organism being tested and sequence reads derived from (or reverse transcribed from) extracted RNA molecules are aligned to the reference genome to determine location, fragment length, and/or start and end positions. For example, in one embodiment, sequence reads are aligned to human reference genome hg19. The sequence of the human reference genome, hg19, is available from Genome Reference Consortium with a reference number, GRCh37/hg19, and also available from Genome Browser provided by Santa Cruz Genomics Institute. The alignment position information may indicate a beginning position and an end position of a region in the reference genome that corresponds to a beginning nucleotide base and end nucleotide base of a given sequence read. Alignment position information may also include sequence read length, which can be determined from the beginning position and end position. A region in the reference genome may be associated with a gene or a segment of a gene.

### Identification of Dark Channel RNA Molecules

Aspects of the disclosure include computer-implemented methods for identifying one or more RNA sequences indicative of a disease state in a subject (or “dark channel RNA molecules”). In some embodiments, the methods involve obtaining, by a computer system, a first set of sequence reads from a plurality of RNA molecules from a first test sample obtained from a subject known to have the disease, wherein the first test sample comprises a plurality of cell-free RNA (cfRNA) molecules, and a second set of sequence reads from a plurality of RNA molecules from a control sample, detecting, one or more RNA sequences that are present in the first set of sequence reads, and that are not present in the second set of sequence reads, to identify one or more RNA sequences that are indicative of the disease state. In some embodiments, the first test sample obtained from the patient comprises a bodily fluid (e.g., blood, plasma, serum, urine, saliva, pleural fluid, pericardial fluid, cerebrospinal fluid (CSF), peritoneal fluid, or any combination thereof). In one preferred embodiment, a test sample obtained from the patient is a plasma sample. In some embodiments, the control sample comprises a plurality of RNA molecules obtained from healthy cells from the subject (e.g., white blood cells).

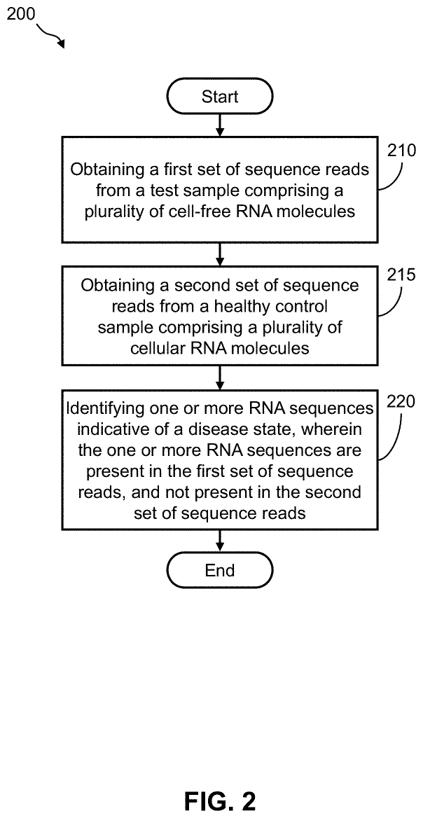

FIG. 2 is a flow diagram illustrating a method for identifying one or more RNA sequences indicative of a disease state, in accordance with one embodiment of the present disclosure. As shown in FIG. 2, at step 210, a first set of sequence reads is obtained from a biological test sample comprising a plurality of cell-free RNA (cfRNA) molecules. The cell-free containing biological test sample can be any a bodily fluid, such as, blood, plasma, serum, urine, pleural fluid, cerebrospinal fluid, tears, saliva, or ascitic fluid. In accordance with this embodiment, the cfRNA biological test sample is obtained from a test subject known to have, or suspected of having a disease, the cfRNA molecules extracted from the sample and sequence reads determined (as described elsewhere herein). For example, in one embodiment, a complementary DNA strand is synthesized using a reverse transcription step generating a cDNA/RNA hybrid molecule, the RNA molecule degraded, a double stranded DNA molecule synthesized from the cDNA strand using a polymerase, a sequencing library prepared, and sequence reads determined using a sequencing platform. The sequencing step can be any carried out using any known sequencing platform in the art, such as, any massively parallel sequencing platform, including a sequencing-by-synthesis platform (e.g., Illumina's HiSeq X) or a sequencing-by-ligation platform (e.g. the Life Technologies SOLiD platform), the Ion Torrent/Ion Proton, semiconductor sequencing, Roche 454, single molecular sequencing platforms (e.g. Helicos, Pacific Biosciences and nanopore), as previously described. Alternatively, other means for detecting and quantifying the sequence reads can be used, for example, array-based hybridization, probe-based in-solution hybridization, ligation-based assays, primer extension reaction assays, can be used to determine sequence reads from DNA molecules (e.g., converted from RNA molecules), as one of skill in the art would readily understand.

At step 220, a second set of sequence reads is obtained from a healthy control sample. In one embodiment, the healthy control sample is from the same subject and comprises a plurality of cellular RNA molecules. For example, the control sample can be blood cells, such as white blood cells, and the plurality of sequence reads derived from RNA molecules extracted from the blood cells. In accordance with this embodiment, the RNA molecules are extracted from the healthy control sample (e.g., blood cells), converted to DNA, a sequencing library prepared, and the second set of sequence reads determined (as described elsewhere herein). In other embodiments, the healthy control sample can be a database of sequence data determined for RNA sequences obtained from a healthy subject, or from healthy cells.

At step 230, sequence reads from the first set of sequence reads and the second set of sequence reads are compared to identifying one or more RNA molecules indicative of a disease state. Moreover, one or more sequence reads (derived from RNA molecules) present in the first set of sequence reads, and not present in the second set of sequence reads, are identified as derived from RNA molecules indicative of a disease state. For example, the first set of sequence reads can comprise sequence reads derived from cfRNA molecules from a plasma sample obtained from a subject known to have, or suspected of having, a disease (e.g., cancer). And the second set of sequence reads can comprise sequence reads derived from RNA molecules from healthy cells (e.g., white blood cells). By comparing, and removing, the second set of sequence reads derived from healthy cells from the first set of sequence reads derived from a cell-free RNA sample, one can identify the sequence reads derived from a disease state (e.g., cancer).

In some embodiments, a control data set of RNA sequences includes a plurality of sequence reads obtained from one or more healthy subjects. In various embodiments, the second set of sequence reads comprises RNA sequence information obtained from a public database. Public databases that can be used in accordance with embodiments of the invention include the tissue RNA-seq database GTEx (available at gtexportal.org/home). In some embodiments, a control data set of RNA sequences includes a plurality of sequence reads obtained from a plurality of blood cells from the subject. For example, in some embodiments, a plurality of sequence reads are obtained from a subject's white blood cells (WBCs).

In embodiments, identification of dark channel RNA molecules is used to select corresponding polypeptide biomarkers.

### Detection of Tumor-Derived RNA Molecules

Aspects of the disclosure include computer-implemented methods for detecting one or more tumor-derived RNA molecules in a subject. In some embodiments, the methods involve: obtaining, by a computer system, a first set of sequence reads from a plurality of RNA molecules from a first test sample from a subject known to have a tumor, wherein the first test sample comprises a plurality of cell-free RNA (cfRNA) molecules; obtaining, by a computer system, a second set of sequence reads from a plurality of RNA molecules from a plurality of blood cells from the subject; and/or detecting, by a computer system, one or more RNA sequences that are present in the first set of sequence reads, and that are not present in the second set of sequence reads, to detect the one or more tumor-derived RNA molecules in the subject.

In some embodiments, the first test sample obtained from the patient comprises blood, plasma, serum, urine, saliva, pleural fluid, pericardial fluid, cerebrospinal fluid (CSF), peritoneal fluid, or any combination thereof. In one preferred embodiment, a test sample obtained from the patient is a plasma sample. In some embodiments, the plurality of blood cells obtained from the subject are white blood cells (WBCs).

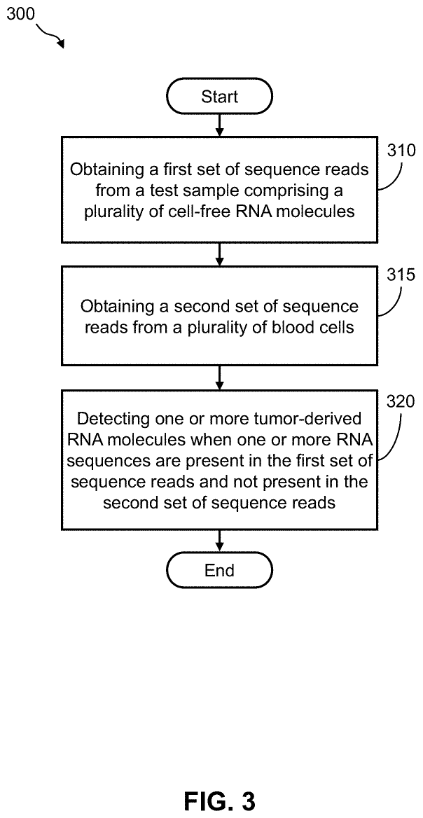

FIG. 3 is a flow diagram illustrating a method for identifying one or more tumor-derived RNA sequences, in accordance with one embodiment of the present invention. At step 310, a first set of sequence reads is obtained from a biological test sample comprising a plurality of cell-free RNA (cfRNA) molecules. In accordance with this embodiment, the cfRNA biological test sample is obtained from a test subject known to have, or suspected of having a disease, the cfRNA molecules extracted from the sample and sequence reads determined (as described elsewhere herein). For example, in one embodiment, a complementary DNA strand is synthesized using a reverse transcription step generating a cDNA/RNA hybrid molecule, the RNA molecule degraded, a double stranded DNA molecule synthesized from the cDNA strand using a polymerase, a sequencing library prepared, and sequence reads determined using a sequencing platform. The sequencing step can be any carried out using any known sequencing platform in the art, as previously described. Alternatively, other means for determining the sequence reads can be used, for example, array-based hybridization, probe-based in-solution hybridization, ligation-based assays, primer extension reaction assays, can be used to detect and/or quantify sequence reads obtained from DNA molecules (e.g., converted from RNA molecules), as one of skill in the art would readily understand.

At step 315, a second set of sequence reads is obtained from blood cells (e.g., white blood cells or buffy coat). In one embodiment, the blood cells are obtained from the same subject and RNA molecules extracted therefrom. In accordance with this embodiment, the RNA molecules are extracted from the blood cells, converted to DNA, a sequencing library prepared, and the second set of sequence reads determined (as described elsewhere herein). In general, any known method in the art can be used to extract and purify cell-free nucleic acids from the test sample. For example, cell-free nucleic acids can be extracted and purified using one or more known commercially available protocols or kits, such as the QIAamp circulating nucleic acid kit (Qiagen).

At step 320, one or more tumor-derived RNA molecules is detected when one or more RNA sequences are present in the first set of sequence reads and not present in the second set of sequence reads. Moreover, one or more sequence reads (derived from RNA molecules) present in the first set of sequence reads, and not present in the second set of sequence reads, are identified as derived from RNA molecules indicative of a disease state. For example, the first set of sequence reads can comprise sequence reads derived from cfRNA molecules from a plasma sample obtained from a subject known to have, or suspected of having, a disease (e.g., cancer). And the second set of sequence reads can comprise sequence reads derived from RNA molecules from blood cells (e.g., white blood cells). By comparing, and removing, the second set of sequence reads derived from blood cells from the first set of sequence reads derived from a cell-free RNA sample, one can identify the sequence reads derived from a tumor.

In some embodiments, tumor-derived target polypeptides are detected instead of, or in addition to, cfRNA molecules. In some embodiments, detection of one of a target polypeptide or corresponding target polynucleotide is used to increase the accuracy of or confidence in detection of the other.

### Detecting a Disease State Using Target Molecules

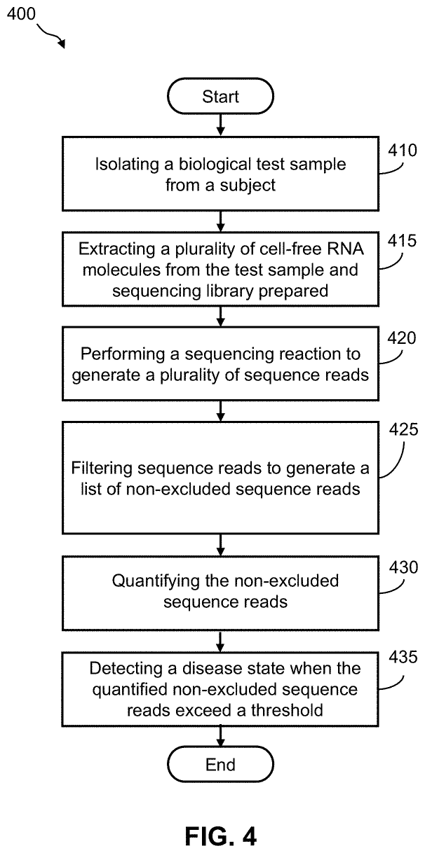

FIG. 4 is a flow diagram illustrating a method for detecting the presence of cancer, determining a state of cancer, monitoring cancer progression, and/or determining cancer type in a subject, in accordance with one embodiment of the present invention. At step 410, a biological test sample is extracted from a subject. As previously described, in one embodiment, the test sample can be a bodily fluid (e.g., blood, plasma, serum, urine, saliva, pleural fluid, pericardial fluid, cerebrospinal fluid (CSF), peritoneal fluid, or any combination thereof) comprising a plurality of cell-free RNA molecules.

At step 415, a plurality of cell-free RNA molecules are extracted from the test sample and a sequencing library prepared. In general, any known method in the art can be used to extract and purify cell-free nucleic acids from the test sample. For example, cell-free nucleic acids (cfRNA molecules) can be extracted and purified using one or more known commercially available protocols or kits, such as the QIAamp circulating nucleic acid kit (Qiagen). After extraction, the cfRNA molecules are used to prepare a sequencing library. In one embodiment, a reverse transcription step is used to produce a plurality of cDNA/RNA hybrid molecules, the RNA strand degraded to produce a single-stranded cDNA molecule, a second strand synthesized to produce a plurality of double-stranded DNA molecules from the single-stranded cDNA molecule templates, and DNA adapters ligated to the plurality of double-stranded DNA molecules to generate a sequencing library. As previously described, the DNA adapters may include one or more sequencing oligonucleotides for use in subsequent cluster generation and/or sequencing (e.g., known P5 and P7 sequences for used in sequencing by synthesis (SBS) (Illumina, San Diego, Calif.)). In another embodiment, the adapter includes a sample specific index sequence, such that, after library preparation, the library can be combined with one or more other libraries prepared from individual samples, thereby allowing for multiplex sequencing. In another embodiment, unique molecular identifiers (UMI) are added through adapter ligation.

At step 420, a sequencing reaction is performed to generate a plurality of sequence reads. In general, any method known in the art can be used to obtain sequence data or sequence reads from the sequencing library. For example, in one embodiment, sequencing data or sequence reads from the sequencing library can be acquired using next generation sequencing (NGS). Next-generation sequencing methods include, for example, sequencing by synthesis technology (Illumina), pyrosequencing (454), ion semiconductor technology (Ion Torrent sequencing), single-molecule real-time sequencing (Pacific Biosciences), sequencing by ligation (SOLiD sequencing), and nanopore sequencing (Oxford Nanopore Technologies). In some embodiments, sequencing is massively parallel sequencing using sequencing-by-synthesis with reversible dye terminators. In other embodiments, sequencing is sequencing-by-ligation. In yet other embodiments, sequencing is single molecule sequencing. In still another embodiment, sequencing is paired-end sequencing. Optionally, an amplification step can be performed prior to sequencing.

At step 425, sequence reads obtained from the cfRNA sample are filtered to generate a list of non-excluded sequence reads and the non-excluded sequence reads quantified at step 430. For example, as described elsewhere herein, the sequence reads obtained from the cfRNA sample can be filtered to exclude sequence known to be present in healthy cells. In one embodiment, RNA molecules extracted from healthy cells (e.g., white blood cells) are sequenced deriving sequence reads that are excluded from the cfRNA derived sequence reads to obtain non-excluded sequence reads. In another embodiment, RNA sequencing data from a database (e.g., a public database) can be used to filter out or exclude sequences known to be present in healthy cells reads comprises to obtain non-excluded sequence reads.

At step 435, a disease state is detected when the quantified non-excluded sequence reads exceed a threshold. In various embodiments, the threshold value is an integer that ranges from about or exactly 1 to about or exactly 10, such as about or exactly 2, 3, 4, 5, 6, 7, 8, or about or exactly 9. In some embodiments, the threshold is a non-integer value, ranging from about or exactly 0.1 to about or exactly 0.9, such as about or exactly 0.2, 0.3, 0.4, 0.5, 0.6, 0.7 or about or exactly 0.8. cfRNA is illustrated in FIG. 4 as an example. In embodiments, the target molecule is a polypeptide (e.g., a polypeptide encoded by a dark channel RNA). Polypeptides may be detected using any of a variety of detection methods, for comparison to a threshold.

Aspects of the disclosure relate to methods for detecting a presence of a cancer, determining a cancer stage, monitoring a cancer progression, and/or determining a cancer type in a subject known to have, or suspected of having a cancer. In some embodiments, the methods involve: (a) quantitatively detecting the presence of one or more target molecules (e.g. polypeptides and/or cfRNA) in a biological fluid of a subject to determine a tumor score; and (b) detecting the presence of the cancer, determining the cancer stage, monitoring the cancer progression, and/or determining the cancer type in the subject when the tumor score exceeds a threshold value. In various embodiments, the threshold value is an integer that ranges from about or exactly 1 to about or exactly 10, such as about or exactly 2, 3, 4, 5, 6, 7, 8, or about or exactly 9. In some embodiments, the threshold is a non-integer value, ranging from about or exactly 0.1 to about or exactly 0.9, such as about or exactly 0.2, 0.3, 0.4, 0.5, 0.6, 0.7 or about or exactly 0.8.

In embodiments where the target molecules comprise polynucleotides (e.g., cfRNA and/or cfDNA), quantitative detection methods in accordance with embodiments of the disclosure can include nucleic acid sequencing procedures, such as next-generation sequencing. In various embodiments, sequencing can involve whole transcriptome sequencing. In various embodiments, sequencing can involve enriching a sample for one or more targeted RNA sequences of interest prior to conducting the sequencing procedure. Alternatively, other means for detecting and quantifying sequence reads can be used, for example, array-based hybridization, probe-based in-solution hybridization, ligation-based assays, primer extension reaction assays, can be used to determine sequence reads from DNA molecules (e.g., converted from RNA molecules), as one of skill in the art would readily understand.

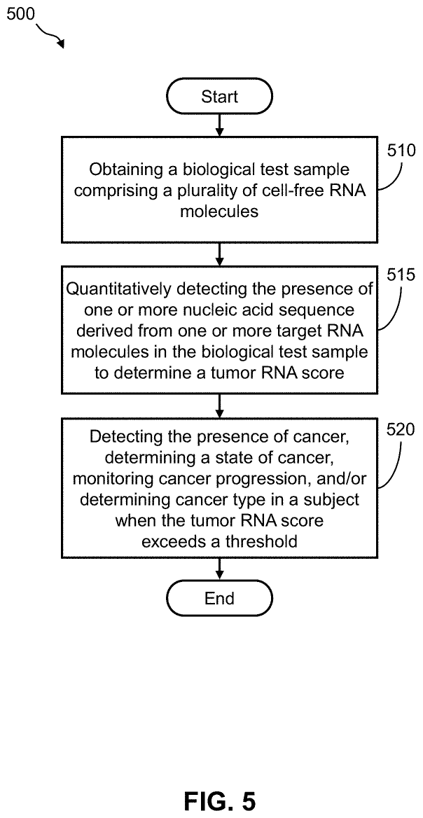

FIG. 5 is a flow diagram illustrating a method for detecting a disease state from one or more sequence reads derived from one or more targeted RNA molecules, in accordance with another embodiment of the present disclosure. At step 510, a biological test sample comprising a plurality of cell-free RNA molecules is obtained. In one embodiment, the biological test sample is a bodily fluid (e.g., a blood, plasma, serum, urine, saliva, pleural fluid, pericardial fluid, cerebrospinal fluid (CSF), peritoneal fluid sample, or any combination thereof).

At step 515, the presence of one or more nucleic acid sequence derived from one or more target RNA molecules in the biological test sample are detected, and quantified, to determine a tumor RNA score. As described elsewhere herein, nucleic acids derived from RNA molecules can be detected and quantified using any known means in the art. For example, in accordance with one embodiment, nucleic acids derived from RNA molecules are detected and quantified using a sequencing procedure, such as a next-generation sequencing platform (e.g., HiSeq or NovaSeq, Illumina, San Diego, Calif.). In other embodiments, nucleic acids derived from RNA molecules are detected and quantified using a microarray, reverse transcription PCR, real-time PCR, quantitative real-time PCR, digital PCR, digital droplet PCR, digital emulsion PCR, multiplex PCR, hybrid capture, oligonucleotide ligation assays, or any combination thereof. As described elsewhere, in one embodiment, cell-free nucleic acids (cfRNA molecules) can be extracted and purified using one or more known commercially available protocols or kits, such as the QIAamp circulating nucleic acid kit (Qiagen). After extraction, the cfRNA molecules are used to prepare a sequencing library. In one embodiment, a reverse transcription step is used to produce a plurality of cDNA/RNA hybrid molecules, the RNA strand degraded to produce a single-stranded cDNA molecule, a second strand synthesized to produce a plurality of double-stranded DNA molecules from the single-stranded cDNA molecule templates. Optionally, in one embodiment, one or more targeted RNA molecules (or DNA molecules derived therefrom) are enriched prior to detection and quantification, as described elsewhere herein. In embodiments, a target polypeptide encoded by the target RNA molecule is detected instead of or in addition to detecting the target RNA molecule, which may similarly be used to determine a tumor score.

In one embodiment, the tumor score is the quantity or count of targeted molecules (or, in the case of polynucleotides, sequence reads obtained from RNA or DNA molecules) detected. In another embodiment, the tumor score comprises a mean, a mode, or an average of the total number of targeted molecules (or, in the case of polynucleotides, sequence reads obtained from RNA or DNA molecules) detected divided by the total number of represented genes targeted for detection. In still other embodiments, the tumor score is determined by inputting the sequence reads into a prediction model, and the tumor score output as a likelihood or probability, as described elsewhere herein.

At step 520, the presence of cancer is detected, a state of cancer determined, cancer progression monitored, and/or a cancer type determined in a subject when the tumor score exceeds a threshold. The threshold value can be an integer that ranges from about or exactly 1 to about or exactly 10, such as about or exactly 2, 3, 4, 5, 6, 7, 8, or about or exactly 9. In some embodiments, the threshold is a non-integer value, ranging from about or exactly 0.1 to about or exactly 0.9, such as about or exactly 0.2, 0.3, 0.4, 0.5, 0.6, 0.7 or about or exactly 0.8. Alternatively, when the tumor score is output from a prediction model, the output can simply be a likelihood or probability indicating the likelihood or probability that the subject has cancer, or a cancer type.

### Cancer Indicator Score

Aspects of the disclosure are directed to computer-implemented methods for detecting the presence of a cancer in a patient. In some embodiments, the methods involve: receiving a data set in a computer comprising a processor and a computer-readable medium, wherein the data set comprises a plurality of sequence reads obtained by sequencing a plurality of nucleic acid molecules (e.g., DNA molecules) derived from a plurality of targeted ribonucleic acid (RNA) molecules in a biological test sample from the patient, and wherein the computer-readable medium comprises instructions that, when executed by the processor, cause the computer to: determine an expression level for the plurality of targeted molecules from the biological test sample; comparing the expression level of each of the targeted molecules to a tissue score matrix to determine a cancer indicator score for each targeted molecule; aggregate the cancer indicator score for each targeted molecule to generate a cancer indicator score for the biological test sample; and detecting the presence of the cancer in the patient when the cancer indicator score for the biological test sample exceeds a threshold value. In embodiments, the expression level is determined from the amount of target polypeptide detected in the sample, either alone or in combination with the level of a target RNA encoding the target polypeptide, or portion thereof. In embodiments, the expression level is determined, at least in part, from cfDNA encoding a target polypeptide or target cfRNA. For example, a copy number of cfDNA for a particular biomarker gene that is above a threshold may indicate increased expression of that gene. In embodiments, two or more of polypeptide, cfRNA, and cfDNA are combined to increase the confidence that a sample genuinely has an increased expression of a given gene. For example, a polypeptide level may be combined with a cfRNA level, and optionally a cfDNA level.

In some embodiments, the target molecules have an expression level in patients with a known cancer status that exceeds their expression level in healthy patients. In certain embodiments, an expression level of a target molecule in a patient with a known cancer status ranges from about or exactly 2 to about or exactly 10 times greater, such as about or exactly 3, 4, 5, 6, 7, 8, or about or exactly 9 times greater, than the expression level of the target molecule in a healthy patient. In various embodiments, a target molecule is not detectable in a biological test sample from a healthy patient, e.g., the target polypeptide and/or target RNA molecule has an undetectable expression level.

In some embodiments, the number of target molecules in the biological test sample ranges from about or exactly 1 to about or exactly 2000, from about or exactly 10 to about or exactly 1000, from about or exactly 10 to about or exactly 500, or from about or exactly 10 to about or exactly 500. In other embodiments, the number of target molecules ranges from about or exactly 1 to about or exactly 50, from about or exactly 1 to about or exactly 40, from about or exactly 1 to about or exactly 30, or from about or exactly 1 to about or exactly 20, such as about or exactly 2, 3, 4, 5, 6, 7, 8, 9, 10, 11, 12, 13, 14, 15, 16, 17, 18, 19, or about or exactly 20. In embodiments, the target molecules are polypeptides. In embodiments, the target molecules are RNA molecules. In embodiments, the target molecules are polypeptides and RNA molecules from the same genes.

In some embodiments, the cancer indicator score comprises an aggregate of the total number of targeted molecules (or, in the case of polynucleotides, sequence reads obtained from RNA or DNA molecules) detected from the biological test sample. In another embodiment, the cancer indicator score comprises a mean, a mode, or an average of the total number of targeted molecules (or sequence reads) detected divided by the total number of represented genes targeted for detection. In still other embodiments, the cancer indicator score is determined by inputting the detection results (e.g., polypeptide detection and/or sequence reads) into a prediction model, and the cancer indicator score is output as a likelihood or probability, as described elsewhere herein.

In some embodiments, the threshold value is an integer that ranges from about or exactly 1 to about or exactly 10, such as about or exactly 2, 3, 4, 5, 6, 7, 8, or about 9. In some embodiments, the threshold is a non-integer value, ranging from about or exactly 0.1 to about or exactly 0.9, such as about or exactly 0.2, 0.3, 0.4, 0.5, 0.6, 0.7 or about or exactly 0.8. In other embodiments, where the target molecule is a target polynucleotide (e.g., RNA), the threshold value ranges from about or exactly 0.5 to about or exactly 5 reads per million (RPM), such as about or exactly 1, 1.5, 2, 2.5, 3, 3.5, 4, or about or exactly 4.5 RPM. The cancer indicator score threshold value can be determined based on the quantity of targeted RNA molecules (or sequence reads derived therefrom) detected in a control sample, for example a healthy subject or a subject with a known disease state. Alternatively, when the cancer indicator score is output from a prediction model, the output can simply be a likelihood or probability indicating the likelihood or probability that the subject has cancer, or a cancer type.

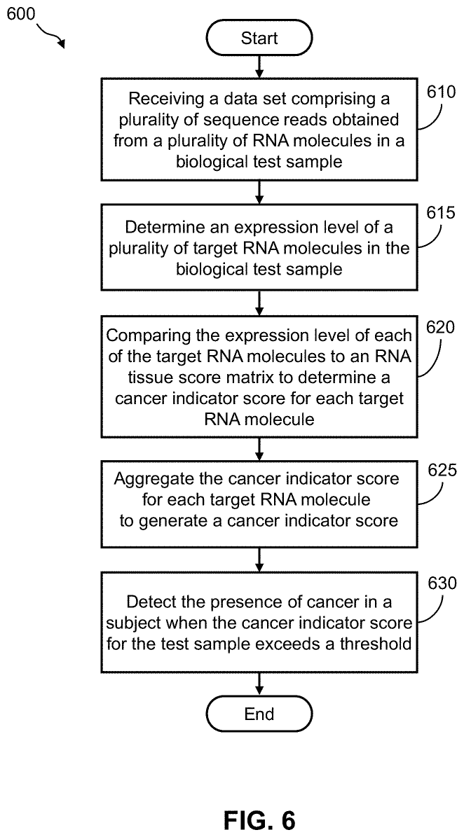

FIG. 6 is a flow diagram illustrating a method for detecting the presence of cancer in a subject based on a cancer indicator score, in accordance with one embodiment of the present disclosure. At step 610, a data set is received comprising a plurality of sequence reads derived from a plurality of cfRNA molecules in a biological test sample. For example, a plurality of sequence reads can be determined for a plurality of cfRNA molecules extracted from a biological test sample, as described herein. Moreover, cfRNA molecules are reverse transcribed to create DNA molecules and the DNA molecules sequenced to generate sequence reads.

At step 615, an expression level is determined for a plurality of target RNA molecules in the biological test sample. For example, in one embodiment, the expression level of targeted RNA molecules can be determined based on quantification of detected sequence reads derived from one or more targeted RNA molecules of interest.

At step 620, the expression level of each of the target RNA molecules is compared to an RNA tissue score matrix to determine a cancer indicator score for each target RNA molecule. The RNA tissue score matrix can be determined from a training set comprising sequence reads derived from a plurality of cancer training samples with known cancer status.

At step 625, the cancer indicator scores for each target RNA molecule are aggregated to generate a cancer indicator score. In some embodiments, the cancer indicator score comprises an aggregate of the total number of targeted RNA molecules (or sequence reads obtained from DNA molecules derived from the targeted RNA molecules) detected from the biological test sample. In another embodiment, the cancer indicator score comprises a mean, a mode, or an average of the total number of targeted RNA molecules (or sequence reads obtained from DNA molecules derived from the targeted RNA molecules) detected divided by the total number of genes from which RNA molecules are targeted.

At step 630, detect the presence of cancer in a subject when the cancer indicator score for the test sample exceeds a threshold. As described above, in one embodiment, the threshold value is an integer that ranges from about or exactly 1 to about or exactly 10, such as about or exactly 2, 3, 4, 5, 6, 7, 8, or about or exactly 9. In some embodiments, the threshold is a non-integer value, ranging from about or exactly 0.1 to about or exactly 0.9, such as about or exactly 0.2, 0.3, 0.4, 0.5, 0.6, 0.7 or about or exactly 0.8. In other embodiments, the threshold value ranges from about or exactly 0.5 to about or exactly 5 reads per million (RPM), such as about or exactly 1, 1.5, 2, 2.5, 3, 3.5, 4, or about or exactly 4.5 RPM.

Aspects of the disclosure include methods for determining a cancer cell type or tissue of origin of the cancer in the patient based on the expression level of one or more of the target molecules, the cancer indicator score for one or more of the target molecules, the cancer indicator score for the biological test sample, or any combination thereof. In various embodiments, the methods further involve therapeutically classifying a patient into one or more of a plurality of treatment categories based on the expression level of one or more of the target molecules, the cancer indicator score for one or more of the target molecules, the cancer indicator score for the biological test sample, or any combination thereof.

In various embodiments, the computer is configured to generate a report that includes an expression level of one or more of the target molecules, a cancer indicator score for one or more of the target molecules, a cancer indicator score for the biological test sample, an indication of the presence or absence of the cancer in the patient, an indication of the cancer cell type of tissue of origin of the cancer in the patient, a therapeutic classification for the patient, or any combination thereof.

### Tissue Matrix Score

Aspects of the disclosure include methods for constructing a tissue score matrix. In some embodiments, the methods involve compiling a plurality of RNA sequence reads obtained from a plurality of patients to generate an RNA expression matrix, and normalizing the RNA expression matrix with a tissue-specific RNA expression matrix to construct the RNA tissue score matrix. In various embodiments, the tissue-specific RNA expression matrix comprises a plurality of reference human tissues. In various embodiments, the RNA sequence reads are obtained from a plurality of healthy patients to construct a healthy RNA tissue score matrix. In various embodiments, the RNA sequence reads are obtained from a plurality of patients having a known cancer type to construct a cancer RNA tissue score matrix. In some embodiments, the methods involve compiling a plurality of detected polypeptide levels obtained for a plurality of patients to generate an expression matrix, and normalizing the expression matrix with a tissue-specific expression matrix to construct a tissue score matrix. In various embodiments, the tissue-specific expression matrix comprises a plurality of reference human tissues. In various embodiments, the detected polypeptide levels are obtained from a plurality of healthy patients to construct a healthy tissue score matrix. In various embodiments, the detected polypeptide levels are obtained from a plurality of patients having a known cancer type to construct a cancer tissue score matrix. In some embodiments, detected polypeptide levels are combined with RNA expression levels to generate a given expression matrix.

### Target Molecules and Analysis Techniques

In some aspects, the present disclosure provides methods of detecting cancer in a subject. Methods in accordance with some embodiments of the disclosure can be performed on polypeptides and/or polynucleotides (e.g., cfRNA molecules and/or ctRNA molecules). In some embodiments, target molecules that are used in the subject methods include target molecules from cancerous and non-cancerous cells. In some embodiments, the target molecules comprise polypeptides. In embodiments, the target molecules comprise polypeptide and one or more of cfRNA and cfDNA.

In embodiments, methods include: (a) measuring a plurality of target molecules in a biological fluid of the subject, wherein the plurality of target molecules are selected from polypeptides of Table 11, and optionally one or more of Tables 8 or 12-19; and (b) detecting the cancer, wherein detecting the cancer comprises detecting one or more of the target molecules above a threshold level. In embodiments, the plurality of target molecules are selected from at least 2, 3, 4, 5, 6, 7, 8, 9, 10, 11, 12, 13, 14, 15, 16, 17, 18, 19, 20, 25, 30, 35, 40, 45, 50, or more polypeptides and/or transcripts of genes listed in one or more of Tables 8, 11-14 or 17. Target molecules can be from genes selected from any one of these tables, or any combination thereof. In embodiments, the number of tables selected from among Tables 8, 11-14, or 17 is 2, 3, 4, or all tables. In embodiments, the target molecules that are measured are from fewer than 500 genes (e.g., fewer than 400, 300, 200, 100, or 50 genes). In embodiments, the target molecules are target polypeptides. In embodiments, target molecules are target polypeptides, and cell free polynucleotides encoding the same (e.g., cfRNA and/or cfDNA). In embodiments, the target molecules comprise cfRNA, and measuring the plurality of cfRNA molecules comprises enriching for the plurality of cfRNA molecules (or cDNA molecules thereof) prior to detection or measurement, such as by sequencing.

In some embodiments, one or more target molecules are derived from one or more genes selected from the genes listed in Table 1. In embodiments, the one or more target molecules are derived from at least 2, 3, 4, 5, 6, 7, 8, 9, 10, 15, 20, 25, or 30 genes from Table 1. In embodiments, the one or more target molecules are derived from at least 5 genes from Table 1. In embodiments, the one or more target molecules are derived from at least 10 genes from Table 1. In embodiments, the one or more target molecules are derived from all of the genes from Table 1. In embodiments, the one or more target molecules are derived from at least one of the first 5 genes of Table 1 (AGR2, HOXC10, S100A7, BPIFA1, and/or ID12-AS1), and optionally one or more additional genes from Table 1. In embodiments, the one or more target molecules includes polypeptides and/or transcripts of the AGR2 gene. In embodiments, the one or more target molecules includes polypeptides and/or transcripts of AGR2, HOXC10, S100A7, BPIFA1, and IDI2-AS1. In embodiments, the target molecules that are measured are from fewer than 500 genes (e.g., fewer than 400, 300, 200, 100, or 50 genes). Table 1 below provides examples of cancer dark channel biomarkers.

TABLE 1

| | | |
|---|---|---|
| AGR2 | HOXC10 | S100A7 |
| BPIFA1 | IDI2-AS1 | SCGB2A2 |
| CASP14 | KRT16P2 | SERPINB5 |
| CSN1S1 | LALBA | SFTA3 |
| DISP2 | LINC00163 | SFTPA2 |
| EIF2D | NKX2-1 | SLC34A2 |
| FABP7 | OPN1SW | TFF1 |
| GABRG1 | PADI3 | VTCN1 |
| GNAT3 | PTPRZ1 | WFDC2 |
| GRHL2 | ROS1 | MUC5B |
| SMIM22 | CXCL17 | RNU1-1 |
| KLK5 | | |

In some embodiments, one or more target molecules are derived from one or more genes selected from the genes listed in Table 2. In embodiments, the one or more target molecules are derived from at least 2, 3, 4, 5, 6, 7, 8, 9, or 10 genes from Table 2. In embodiments, the one or more target molecules are derived from at least 5 genes from Table 2. In embodiments, the one or more target molecules are derived from at least 10 genes from Table 2. In embodiments, the one or more target molecules are derived from all of the genes from Table 2. In embodiments, the one or more target molecules are derived from at least one of the first 5 genes of Table 2 (ROS1, NKX2-1, GGTLC1, SLC34A2, and SFTPA2), and optionally one or more additional genes from Table 2. In embodiments, the one or more target molecules include polypeptides and/or transcripts of the ROS1 gene. In embodiments, the one or more target molecules include polypeptides and/or transcripts of ROS1, NKX2-1, GGTLC1, SLC34A2, and SFTPA2. In embodiments, the target molecules that are measured are from fewer than 500 genes (e.g., fewer than 400, 300, 200, 100, or 50 genes). Table 2 below provides examples of dark channel lung cancer biomarkers.

TABLE 2

ROS1
NKX2-1
GGTLC1
SLC34A2
SFTPA2
BPIFA1
SFTA3
GABRG1
AGR2
GNAT3
MUC5B
SMIM22
CXCL17
WFDC2

In some embodiments, one or more target molecules are derived from one or more genes selected from the genes listed in Table 3. In embodiments, the one or more target molecules are derived from at least 2, 3, 4, 5, 6, 7, 8, or 9 genes from Table 3. In embodiments, the one or more target molecules are derived from at least 5 genes from Table 3. In embodiments, the one or more target molecules are derived from all of the genes from Table 3. In embodiments, the one or more target molecules are derived from at least one of the first 5 genes of Table 3 (SCGB2A2, CSN1S1, VTCN1, FABP7, and LALBA), and optionally one or more additional genes from Table 3. In embodiments, the one or more target molecules include polypeptides and/or transcripts of the SCGB2A2 gene. In embodiments, the one or more target molecules include polypeptides and/or transcripts of SCGB2A2, CSN1S1, VTCN1, FABP7, and LALBA. In embodiments, the target molecules that are measured are from fewer than 500 genes (e.g., fewer than 400, 300, 200, 100, or 50 genes). Table 3 below provides examples of breast cancer dark channel biomarkers.

TABLE 3

SCGB2A2
CSN1S1
VTCN1
FABP7
LALBA
CASP14
KLK5
WFDC2
OPN1SW

In some embodiments, one or more target molecules are derived from one or more genes selected from the genes listed in Table 4. In embodiments, the one or more target molecules are derived from at least 2, 3, 4, or 5 genes from Table 4. In embodiments, the one or more target molecules are derived from at least 5 genes from Table 4. In embodiments, the one or more target molecules are derived from all of the genes from Table 4. In embodiments, the one or more target molecules are derived from at least one of the first 5 genes of Table 4 (CASP14, CRABP2, FABP7, SCGB2A2, and SERPINB5), and optionally one or more additional genes from Table 4. In embodiments, the one or more target molecules include polypeptides and/or transcripts of the CASP14 gene. In embodiments, the one or more target molecules include polypeptides and/or transcripts of CASP14, CRABP2, FABP7, SCGB2A2, and SERPINB5. In embodiments, the target molecules that are measured are from fewer than 500 genes (e.g., fewer than 400, 300, 200, 100, or 50 genes). Table 4 below provides examples of breast cancer biomarkers identified using a heteroDE method, as described herein.

TABLE 4

CASP14
CRABP2
FABP7
SCGB2A2
SERPINB5
TRGV10
VGLL1
TFF1
AC007563.5

In some embodiments, one or more target molecules are derived from one or more genes selected from the genes listed in Table 5. In embodiments, the one or more target molecules are derived from at least 2, 3, 4, 5, 6, 7, 8, 9, 10, 15, 20, or 25 genes from Table 5. In embodiments, the one or more target molecules are derived from at least 5 genes from Table 5. In embodiments, the one or more target molecules are derived from at least 10 genes from Table 5. In embodiments, the one or more target molecules includes all of the genes from Table 5. In embodiments, the one or more target molecules are derived from at least one of the first 5 genes of Table 5 (PTPRZ1, AGR2, SHANK1, PON1, and MYO16_AS1), and optionally one or more additional genes from Table 5. In embodiments, the one or more target molecules include polypeptides and/or transcripts of the PTPRZ1 gene. In embodiments, the one or more target molecules include polypeptides and/or transcripts of PTPRZ1, AGR2, SHANK1, PON1, and MYO16_AS1. In embodiments, the target molecules that are measured are from fewer than 500 genes (e.g., fewer than 400, 300, 200, 100, or 50 genes). Table 5 below provides examples of lung cancer biomarkers identified using an information gain method, as described herein.

TABLE 5

| | | |
|---|---|---|
| PTPRZ1 | AGR2 | SHANK1 |
| PON1 | MYO16_AS1 | NPAS3 |
| LINC00407 | LMO3 | KRT15 |
| ELFN2 | MUC5B | SAA2 |
| SLIT3 | NALCN | LUM |
| GDA | LINC01498 | TMEM178A |
| RCVRN | XKRX | ROS1 |
| NBPF7 | ACSM5 | SLC10A3 |
| SAA1 | CYP3A4 | LINC00643 |
| GLP1R | TRAV8_5 | GNAT3 |

In some embodiments, one or more target molecules are derived from one or more genes selected from the genes listed in Table 6. In embodiments, the one or more target molecules are derived from at least 2, 3, 4, 5, 6, 7, 8, 9, 10, 15, 20, or 25 genes from Table 6. In embodiments, the one or more target molecules are derived from at least 5 genes from Table 6. In embodiments, the one or more target molecules are derived from at least 10 genes from Table 6. In embodiments, the one or more target molecules are derived from all of the genes from Table 6. In embodiments, the one or more target molecules are derived from at least one of the first 5 genes of Table 6 (ADARB2, HORMAD2, SPDYE18, RPS19, and CYP4F35P), and optionally one or more additional genes from Table 6. In embodiments, the one or more target molecules include polypeptides and/or transcripts of the ADARB2 gene. In embodiments, the one or more target molecules include polypeptides and/or transcripts of ADARB2, HORMAD2, SPDYE18, RPS19, and CYP4F35P. In embodiments, the target molecules that are measured are from fewer than 500 genes (e.g., fewer than 400, 300, 200, 100, or 50 genes). Table 6 below provides examples of breast cancer biomarkers identified using an information gain method, as described herein.

TABLE 6

| | | |
|---|---|---|
| ADARB2 | HORMAD2 | SPDYE18 |
| RPS19 | CYP4F35P | MIR503HG |
| SLC34A2 | MUC5B | IGKVID_16 |
| TLX2 | IDI2 | PDPK2P |
| ACTBP2 | TTPA | LINC01140 |
| RIMKLA | WNT6 | TRBV6_4 |
| RANBP6 | FHOD3 | LINC00856 |
| CTF1 | GSTA9P | FOXC1 |
| FAM9C | SMIM2_AS1 | CCDC188 |
| FAM171A2 | GRIA2 | GABRR2 |

In some embodiments, one or more target molecules are derived from one or more genes selected from the genes listed in Table 7. In embodiments, the one or more target molecules are derived from at least 2, 3, 4, 5, 6, 7, 8, 9, or 10 genes from Table 7. In embodiments, the one or more target molecules are derived from at least 5 genes from Table 7. In embodiments, the one or more target molecules are derived from at least 10 genes from Table 7. In embodiments, the one or more target molecules are derived from all of the genes from Table 7. In embodiments, the one or more target molecules are derived from at least one of the first 5 genes of Table 7 (S100A7, FOXA1, BARX2, MMP7, and PLEKHG4B), and optionally one or more additional genes from Table 7. In embodiments, the one or more target molecules include polypeptides and/or transcripts of the S100A7 gene. In embodiments, the one or more target molecules include polypeptides and/or transcripts of S100A7, FOXA1, BARX2, MMP7, and PLEKHG4B. In embodiments, the target molecules that are measured are from fewer than 500 genes (e.g., fewer than 400, 300, 200, 100, or 50 genes). Table 7 below provides examples of dark channel cancer biomarkers that are expressed at relatively high levels in cancer tissue.

TABLE 7

| | | |
|---|---|---|
| S100A7 | FOXA1 | BARX2 |
| MMP7 | PLEKHG4B | TFAP2A |
| TOX3 | VTCN1 | ANKRD30A |
| COL22A1 | FDCSP | LAMA1 |
| MATN3 | TFF1 | VGLL1 |

In some embodiments, one or more target molecules are derived from one or more genes selected from the genes listed in Table 11. In embodiments, the one or more target molecules are derived from at least 2, 3, 4, 5, 10, 25, 50, 100, 150, 200, 300, or 400 genes from Table 11. In embodiments, the one or more target molecules are derived from at least 5 genes from Table 11. In embodiments, the one or more target molecules are derived from at least 25 genes from Table 11. In embodiments, the one or more target molecules are derived from at least 100 genes from Table 11. In embodiments, the one or more target molecules are derived from at least 200 genes from Table 11. In embodiments, the one or more target molecules are derived from at least 300 genes from Table 11. In embodiments, the one or more target molecules are derived from all of the genes from Table 11. In embodiments, the target molecules that are measured are from fewer than 500 genes (e.g., fewer than 400, 300, 200, 100, or 50 genes). Table 11 below provides examples of cancer biomarkers.

TABLE 11

AARD
ABCA12
ABCC11
ABCC8
ACTL8
ADAMTS15
ADAMTS8
ADGRF1
ADH7
ADIPOQ
ADRB1
AFP
AGER
AGR3
AGTR1
AGTR2
AIF1
AKR1B15
ALG9
ALK
ALPG
ALPP
ANKRD30B
ANKRD35
ANXA8
AQP4
ARHGEF38
ARL14
ART3
ATF6B
ATP10B
ATP6V0A4
ATP7A
AZGP1P1
B3GNT3
B3GNT6
BMP5
BPIFA2
BPIFB2
BRINP2
BRINP3
C2CD4A
C4B
C5orf30
C5orf46
C5orf49
C9orf116
C9orf152
CA12
CACNG1
CACNG4
CALCA
CALML5
CCDC125
CCDC160
CCKBR
CCNO
CCT8P1
CD99
CDH3
CDKL2
CEACAM5
CEP41
CFTR
CGA
CHGA
CHIA
CHRM1
CHST9
CIDEA
CKMT1A
CKMT1B
CLCA2
CLDN10
CLDN18
CLDN6
CLEC3A
CLIC1
CLPSL1
CLPSL2
CLSTN2
CNGA3
CNOT3
COL6A5
COL6A6
COPG2
CRYAB
CSF2
CSN3
CSNK2B
CST1
CST9
CT62
CTSE
CWC15
CYP21A2
CYP27C1
CYP4F23P
CYP4F8
CYP4Z2P
DCX
DHX16
DIXDC1
DLX1
DLX3
DMRTA2
DNAJB13
DOC2A
DSCAM-AS1
DSTNP2
DUOXA1
DXO
DYDC2
ECEL1
EGFR
EHMT2
ELF5
EMX1
EN1
EPN3
EPOP
EPYC
ERBB2
ERBB4
ERN2
ERVH48-1
ESR1
ESRP1
ESYT3
ETV3L
EXTL1
F7
FAM198A
FAM19A3
FAM216B
FAXC
FBN3
FBXL19-AS1
FEZF1-AS1
FGFBP1
FGFR1
FGFR2
FGFR3
FGFR4
FKBP10
FKBPL
FLRT3
FOLR1
FOXA2
FOXI1
FOXJ1
FOXQ1
FUT2
FUT3
FZD7
FZD9
GAL3ST1
GATA3-AS1
GCNT3
GCNT4
GDF15
GFRA1
GGT6
GIN1
GJB5
GJC3
GKN2
GNG4
GNL1
GP2
GPR12
GPR143
GPR39
GPR87
GPSM3
GPX2
HAPLN1
HHIP
HOTAIR
HOXB13
HOXC11
HOXC13
HOXC6
HOXC8
HOXC9
HS6ST3
IGF2BP1
IGSF1
IL20
INHA
INSL4
IP6K3
IRX1
IRX2
IRX4
IRX5
ITGA10
ITGB6
ITIH6
IVL
KCNC2
KCNJ11
KCNJ3
KCNK15
KCNK2
KCNK3
KIFC1
KLHL38
KLK8
KRAS
KRT14
KRT23
KRT6B
KRT83
LAMA4
LDLRAD1
LEMD1
LENG1
LEP
LILRB1
LINC00052
LINC00261
LINC00511
LINC00641
LINC00707
LINC00993
LINC01016
LINC01087
LMX1B
LRRC31
LRRN4
LY6D
MAGEA3
MAGEA6
MAPT
MB
MBOAT7
MET
MEX3A
MIA
MICA
MILR1
MIR205HG
MKX
MMP10
MMP12
MOCS3
MSLNL
MSX2
MTM1
MUC15
MUC21
MUC6
MUCL1
MUCL3
NCMAP
NDNF
NDUFA3
NELL1
NKAIN1
NKAIN4
NKX1-2
NKX2-1-AS1
NKX2-2
NKX6-1
NNAT
NTRK1
NXNL2
OBP2B
ODAM
OSCAR
OVOL2
PAEP
PAX7
PCP4L1
PDE4C
PFDN6
PGC
PGR
PIK3CA
PIP
PITX2
PLA2G12B
PLA2G1B
PLA2G4E
POPDC3
POTEKP
POU5F1
PPIP5K2
PPP1R11
PPP1R14BP3
PPP1R14D
PPP1R18
PRIMA1
PRND
PRR15
PRR15L
PRSS8
PSMB8
PTCHD1
PYDC1
RAB6C
RABL2B
RASD2
RBBP8NL
RET
RGMA
RHOV
RHPN1-AS1
ROPN1
ROPN1B
RRAD
RTL8B
RTN4RL1
RXRB
S100A1
S100A7A
SCARNA5
SCGB1A1
SCGB1D2
SCGB2A1
SCN7A
SCNN1G
SCTR
SEC14L6
SEMA3B
SERPINA11
SERPINB4
SERPINB7
SEZ6
SEZ6L2
SFRP1
SFRP5
SFTA1P
SFTA2
SFTPA1
SFTPB
SHH
SHISA3
SHISA9
SIM2
SIX4
SLC16A6
SLC22A31
SLC25A48
SLC26A3
SLC26A9
SLC37A4
SLC44A4
SLC6A14
SLC6A17
SLC9A6
SLITRK4
SLITRK6
SMIM17
SMR3A
SNTN
SOWAHA
SOX2
SOX21
SOX9
SP8
SPDEF
SPINK8
SPON1
SPRR2D
SRSF12
STAC2
STC2
STK32A
STMND1
STOML3
SUN3
SURF6
SUV39H1
SYNM
SYP
SYT9
TAF15
TAP2
TBX4
TCF21
TFAP2B
TMC3
TMEM125
TMEM198
TMEM59L
TMSB15A
TNRC18P1
TRARG1
TRH
TRIM27
TRIM31
TRIM39
TRIM48
TRPV6
TSEN34
TSPY26P
TTC30B
TTC36
TTC6
TTYH1
UCN3
UCP1
UGT2B15
UPK1A
USP41
VARS
VN1R53P
VSTM2A
WFDC10B
WNT3A
YBX1P10
ZBTB12
ZFP57
ZNF737
ZNRD1

In some embodiments, one or more target molecules are derived from one or more genes selected from the genes listed in Table 12. In embodiments, the one or more target molecules are derived from at least 2, 3, 4, 5, 10, 20, 30, 40, 50, or 60 genes from Table 12. In embodiments, the one or more target molecules are derived from at least 5 genes from Table 12. In embodiments, the one or more target molecules are derived from at least 10 genes from Table 12. In embodiments, the one or more target molecules are derived from at least 25 genes from Table 12. In embodiments, the one or more target molecules are derived from at least 50 genes from Table 12. In embodiments, the one or more target molecules are derived from all of the genes from Table 12. In embodiments, the target molecules that are measured are from fewer than 500 genes (e.g., fewer than 400, 300, 200, 100, or 50 genes). Table 12 below provides examples of lung cancer biomarkers.

TABLE 12

AGTR2
AQP4
ATP10B
B3GNT3
B3GNT6
BMP5
BPIFA1
CALCA
CCT8P1
CDKL2
CEACAM5
CFTR
CHIA
COL6A5
CRYAB
CTSE
DSTNP2
EPN3
ERN2
ESYT3
FLRT3
FOXA2
FZD7
GAL3ST1
GCNT3
GDF15
GFRA1
GKN2
GPR39
HHIP
IRX5
KRT14
KRT23
KRT6B
LINC00261
LRRN4
MET
MMP12
MUC21
NDNF
NKX2-1-AS1
PCP4L1
PDE4C
PLA2G4E
RASD2
RRAD
RTN4RL1
SCGB1A1
SCN7A
SCTR
SEC14L6
SFRP1
SFTA1P
SFTA2
SFTPA1
SFTPB
SHH
SHISA3
SLC22A31
SLC26A9
SLC6A14
SLC9A6
SOX2
SOX9
STC2
STK32A
SYNM
TBX4
TCF21
UCN3

In some embodiments, one or more target molecules are derived from one or more genes selected from the genes listed in Table 18. In embodiments, the one or more target molecules are derived from at least 2, 3, 4, 5, 10, 15 or, 19 genes from Table 18. In embodiments, the one or more target molecules are derived from at least 5 genes from Table 18. In embodiments, the one or more target molecules are derived from at least 10 genes from Table 18. In embodiments, the one or more target molecules are derived from at least 15 genes from Table 18. In embodiments, the one or more target molecules are derived from all of the genes from Table 18. In embodiments, the target molecules that are measured are from fewer than 500 genes (e.g., fewer than 400, 300, 200, 100, or 50 genes). Table 18 below provides examples of lung cancer biomarkers identified using a PEA assay method for detecting proteins in plasma samples, as described herein.

TABLE 18

WFDC2
CXCL17
MMP12
GDF15
CEACAM5
PRSS8
TFF1
CWC15
ALPP
GP2
INSL4
CHGA
GFRA1
AGR2
SPON1
DXO
AIF1
FKBPL
SFTPA2
FOLR1

In various embodiments, one or more target molecules are derived from one or more genes selected from the genes listed in Table 13. In embodiments, the one or more target molecules includes at least 2, 3, 4, 5, 10, 20, 30, 40, 50, 60, or 70 genes from Table 13. In embodiments, the one or more target molecules are derived from at least 5 genes from Table 13. In embodiments, the one or more target molecules are derived from at least 10 genes from Table 13. In embodiments, the one or more target molecules are derived from at least 25 genes from Table 13. In embodiments, the one or more target molecules are derived from at least 50 genes from Table 13. In embodiments, the one or more target molecules are derived from all of the genes from Table 13. In embodiments, the target molecules that are measured are from fewer than 500 genes (e.g., fewer than 400, 300, 200, 100, or 50 genes). Table 13 below provides examples of breast cancer biomarkers.

TABLE 13

ABCC8
ADAMTS15
AGR3
ART3
AZGP1P1
B3GNT3
BPIFB2
C2CD4A
CA12
CCDC125
CDH3
CEACAM5
CLSTN2
CRABP2
CSN3
DSCAM-AS1
ELF5
ERBB2
ERBB4
ESR1
EXTL1
F7
FBXL19-AS1
FOLR1
FOXA1
FOXJ1
FUT3
GIN1
GP2
HOXC13
HOXC6
HOXC9
IRX1
IRX5
ITIH6
KCNJ11
KCNK15
KIFC1
KRT23
KRT6B
LINC00511
LINC01087
LMX1B
LY6D
MAPT
MB
MET
MEX3A
MMP12
MSX2
OBP2B
PIK3CA
PPP1R18
PRR15
RET
RGMA
RHPN1-AS1
ROPN1B
RTN4RL1
S100A1
SEMA3B
SFRP1
SLC16A6
SLC44A4
SOWAHA
SOX9
SPDEF
STAC2
STMND1
TRH
TRPV6
TTC6
TTYH1
VARS
VGLL1

In some embodiments, one or more target molecules are derived from one or more genes selected from the genes listed in Table 19. In embodiments, the one or more target molecules are derived from at least 2, 3, 4, 5, 10 or 12 genes from Table 19. In embodiments, the one or more target molecules are derived from at least 5 genes from Table 19. In embodiments, the one or more target molecules are derived from at least 10 genes from Table 19. In embodiments, the one or more target molecules are derived from all of the genes from Table 19. In embodiments, the target molecules that are measured are from fewer than 500 genes (e.g., fewer than 400, 300, 200, 100, or 50 genes). Table 19 below provides examples of breast cancer biomarkers identified using a PEA assay method for detecting proteins in plasma samples, as described herein.

TABLE 19

ADAMTS15
LEP
ERBB2
ERBB4
CGA
AFP
F7
BPIFB2
SFRP1
FGFBP1
LAMA4
GP2
MIA
FGFR2
VTCN1

In some embodiments, one or more target molecules are derived from one or more genes selected from the genes listed in Table 14. In embodiments, the one or more target molecules are derived from at least 2, 3, 4, 5, 10, 15, 20, or 30 genes from Table 14. In embodiments, the one or more target molecules are derived from at least 5 genes from Table 14. In embodiments, the one or more target molecules are derived from at least 10 genes from Table 14. In embodiments, the one or more target molecules are derived from at least 25 genes from Table 14. In embodiments, the one or more target molecules are derived from all of the genes from Table 14. In embodiments, the target molecules that are measured are from fewer than 500 genes (e.g., fewer than 400, 300, 200, 100, or 50 genes). In embodiments, the plurality of target molecules detected above a threshold are molecules derived from a plurality of genes selected from the group consisting of: ADIPOQ, AGR3, ANKRD30A, AQP4, BPIFA1, CA12, CEACAM5, CFTR, CXCL17, CYP4F8, FABP7, FOXI1, GGTLC1, GP2, IL20, ITIH6, LDLRAD1, LEMD1, LMX1B, MMP7, NKAIN1, NKX2-1, ROPN1, ROS1, SCGB1D2, SCGB2A2, SFTA2, SFTA3, SLC34A2, SOX9, STK32A, STMND1, TFAP2A, TFAP2B, TFF1, TRPV6, VGLL1, and VTCN1. Table 14 below provides examples of highly informative cancer biomarkers.

TABLE 14

ADIPOQ
AGR3
ANKRD30A
AQP4
BPIFA1
CA12
CEACAM5
CFTR
CXCL17
CYP4F8
FABP7
FOXI1
GGTLC1
GP2
IL20
ITIH6
LDLRAD1
LEMD1
LMX1B
MMP7
NKAIN1
NKX2-1
ROPN1
ROS1
SCGB1D2
SCGB2A2
SFTA2
SFTA3
SLC34A2
SOX9
STK32A
STMND1
TFAP2A
TFAP2B
TFF1
TRPV6
VGLL1
VTCN1

In some embodiments, one or more target molecules are derived from one or more genes selected from the genes listed in Table 15. In embodiments, the one or more target molecules are derived from at least 2, 3, 4, 5, 10, 25, 50, 100, 150, 200, 300, or 400 genes from Table 15. In embodiments, the one or more target molecules are derived from at least 5 genes from Table 15. In embodiments, the one or more target molecules are derived from at least 25 genes from Table 15. In embodiments, the one or more target molecules are derived from at least 100 genes from Table 15. In embodiments, the one or more target molecules are derived from at least 200 genes from Table 15. In embodiments, the one or more target molecules are derived from at least 300 genes from Table 15. In embodiments, the one or more target molecules are derived from all of the genes from Table 15. In embodiments, the target molecules that are measured are from fewer than 500 genes (e.g., fewer than 400, 300, 200, 100, or 50 genes).

TABLE 15

| | | | | | |
|---|---|---|---|---|---|
| RNU1-1 | ESYT3 | DXO | SFTA1P | NKX2-1 | FBN3 |
| PADI3 | CLSTN2 | C4B | MKX | NKX2-1-AS1 | CASP14 |
| ACTL8 | AGTR1 | CYP21A2 | ANKRD30A | FOXA1 | CYP4F23P |
| PAX7 | GPR87 | ATF6B | LINC00993 | TTC6 | CYP4F8 |
| NCMAP | ARL14 | FKBPL | VN1R53P | SIX4 | B3GNT3 |
| EXTL1 | LRRC31 | AGER | RET | GPX2 | PDE4C |
| NKAIN1 | PIK3CA | GPSM3 | ANXA8 | CHGA | GDF15 |
| GJB5 | SOX2 | TAP2 | PLA2G12B | PRIMA1 | TMEM59L |
| TMEM125 | ADIPOQ | PSMB8 | SFTPA2 | SERPINA11 | ZNF737 |
| CYP4Z2P | FGFR3 | RXRB | SFTPA1 | DISP2 | UPK1A |
| DMRTA2 | FGFBP1 | PFDN6 | DYDC2 | PPP1R14D | MIA |
| LDLRAD1 | SLC34A2 | KIFC1 | SFRP5 | RHOV | CEACAM5 |
| CLCA2 | SHISA3 | IP6K3 | ADRB1 | PLA2G4E | CXCL17 |
| SLC6A17 | GABRG1 | LINC01016 | GFRA1 | CKMT1B | FUT2 |
| CHIA | UGT2B15 | SPDEF | FGFR2 | CKMT1A | KLK5 |
| FAM19A3 | CSN1S1 | CLPSL2 | NKX1-2 | DUOXA1 | KLK8 |
| VTCN1 | ODAM | CLPSL1 | MUC6 | GCNT3 | OSCAR |
| ITGA10 | CSN3 | PGC | MUC5B | C2CD4A | NDUFA3 |
| ANKRD35 | SMR3A | ADGRF1 | CCKBR | CA12 | CNOT3 |
| CCT8P1 | AFP | TFAP2B | SYT9 | CT62 | LENG1 |
| IVL | CDKL2 | BMP5 | SPON1 | TMC3 | MBOAT7 |
| SPRR2D | ART3 | CGA | CALCA | LINC00052 | TSEN34 |
| S100A7A | NKX6-1 | SRSF12 | KCNJ11 | RGMA | TTYH1 |
| S100A7 | ADH7 | FAXC | ABCC8 | SYNM | LILRB1 |
| S100A1 | ARHGEF38 | POPDC3 | SAA2 | MSLNL | SMIM17 |
| MEX3A | PITX2 | LAMA4 | SAA1 | CLDN6 | PRND |
| CRABP2 | NDNF | ROS1 | NELL1 | SMIM22 | LRRN4 |
| NTRK1 | PPP1R14BP3 | FABP7 | MUC15 | SHISA9 | FLRT3 |
| ETV3L | UCP1 | TCF21 | ELF5 | GP2 | OVOL2 |
| PCP4L1 | TNRC18P1 | ESR1 | TRIM48 | SCNN1G | NKX2-2 |
| BRINP2 | HHIP | AGR2 | SCGB2A1 | ERN2 | LINC00261 |
| BRINP3 | GRIA2 | AGR3 | SCGB1D2 | SEZ6L2 | FOXA2 |
| LEMD1 | IRX4 | SP8 | SCGB2A2 | DOC2A | CST9 |
| SLC26A9 | IRX2 | PRR15 | SCGB1A1 | FBXL19-AS1 | CST1 |
| CTSE | IRX1 | SUN3 | CHRM1 | PRSS8 | GGTLC1 |
| EIF2D | C5orf49 | VSTM2A | FOLR1 | PYDC1 | TSPY26P |
| IL20 | CCNO | EGFR | DNAJB13 | ABCC11 | BPIFB2 |
| MIR205HG | CCDC125 | FZD9 | B3GNT6 | TOX3 | BPIFA2 |
| KCNK2 | GCNT4 | GNAT3 | CWC15 | IRX5 | BPIFA1 |
| WNT3A | HAPLN1 | GJC3 | PGR | RRAD | NNAT |
| GNG4 | GIN1 | AZGP1P1 | MMP7 | CDH3 | KCNK15 |
| KCNK3 | PPIP5K2 | SLC26A3 | MMP10 | CLEC3A | WFDC2 |
| ALK | C5orf30 | MET | MMP12 | SLC22A31 | WFDC10B |
| GKN2 | CSF2 | CFTR | ALG9 | TRARG1 | MOCS3 |
| EMX1 | SOWAHA | PTPRZ1 | CRYAB | RTN4RL1 | RBBP8NL |
| SFTPB | SLC25A48 | FEZF1-AS1 | DIXDC1 | GGT6 | NKAIN4 |
| CNGA3 | STK32A | LEP | TTC36 | KRT16P2 | SIM2 |
| EN1 | C5orf46 | OPN1SW | SLC37A4 | SEZ6 | DSCAM-AS1 |
| SCTR | ATP10B | CEP41 | BARX2 | TAF15 | TFF1 |
| CYP27C1 | FOXI1 | COPG2 | ADAMTS8 | EPOP | ERVH48-1 |
| RAB6C | STC2 | AKR1B10 | ADAMTS15 | STAC2 | USP41 |
| POTEKP | MSX2 | AKR1B15 | DSTNP2 | ERBB2 | SEC14L6 |
| LINC01087 | FGFR4 | ATP6V0A4 | LMO3 | KRT23 | GAL3ST1 |
| GPR39 | FOXQ1 | TRPV6 | KRAS | KRT15 | RASD2 |
| KCNJ3 | FOXC1 | PIP | LALBA | KRT14 | MB |
| ITGB6 | TFAP2A | SHH | KRT83 | FKBP10 | ELFN2 |
| SCN7A | STMND1 | FGFR1 | KRT6B | MAPT | RABL2B |
| DLX1 | TRIM27 | SFRP1 | HOXC13 | PRR15L | CD99 |
| TTC30B | ZFP57 | ESRP1 | HOTAIR | HOXB13 | GPR143 |
| FZD7 | ZNRD1 | GRHL2 | HOXC11 | IGF2BP1 | PTCHD1 |
| ERBB4 | PPP1R11 | AARD | HOXC10 | DLX3 | SUV39H1 |
| ABCA12 | TRIM31 | KLHL38 | HOXC6 | EPN3 | SYP |
| TMEM198 | TRIM39 | COL22A1 | HOXC9 | TBX4 | ITIH6 |
| INHA | GNL1 | LY6D | HOXC8 | MILR1 | ATP7A |
| ALPP | DHX16 | RHPN1-AS1 | MUCL1 | CACNG4 | TMSB15A |
| ALPG | PPP1R18 | INSL4 | KCNC2 | CACNG1 | DCX |
| ECEL1 | SFTA2 | YBX1P10 | EPYC | SLC16A6 | AGTR2 |
| SCARNA5 | MUCL3 | NXNL2 | PLA2G1B | SOX9 | SLC6A14 |
| FAM198A | MUC21 | C9orf152 | GPR12 | LINC00511 | IGSF1 |
| SPINK8 | POU5F1 | LMX1B | STOML3 | FOXJ1 | CCDC160 |
| SEMA3B | MICA | OBP2B | FAM216B | CIDEA | RTL8B |
| SNTN | AIF1 | SURF6 | SLITRK6 | ANKRD30B | SLC9A6 |
| ROPN1 | CSNK2B | C9orf116 | SOX21 | AQP4 | VGLL1 |
| ROPN1B | CLIC1 | PAEP | CLDN10 | CHST9 | SLITRK4 |
| TRH | VARS | UCN3 | HS6ST3 | SERPINB5 | MTM1 |
| COL6A5 | SLC44A4 | CALML5 | F7 | SERPINB4 | MAGEA6 |
| COL6A6 | EHMT2 | LINC00707 | LINC00641 | SERPINB7 | MAGEA3 |
| CLDN18 | ZBTB12 | GATA3-AS1 | SFTA3 | FUT3 | |

In embodiments, one or more target molecules are derived from one or more genes selected from one or more of Tables 8 or 11-14 (e.g., 2, 3, 5, or more genes) in combination with one or more genes selected from one or more of Tables 1-6 (e.g., 2, 3, 5, or more genes). In embodiments, one or more target molecules are derived from one or more genes selected from one or more of Tables 8 or 11-14 (e.g., 2, 3, 5, or more genes) in combination with one or more genes selected from Tables 7 (e.g., 2, 3, 5, or more genes). In embodiments, the table selected from Tables 8 or 11-14 is Table 11. In embodiments, the table selected from Tables 8 or 11-14 is Table 12. In embodiments, the table selected from Tables 8 or 11-14 is Table 13. In embodiments, the table selected from Tables 8 or 11-14 is Table 14. In embodiments, the table selected from Tables 8 or 11-14 is Table 8. In embodiments, selection of genes from first and second tables comprises selecting one or more genes in both of the first and second tables. In embodiments, selection of genes from first and second tables comprises selecting one or more genes from the first table that are not in the second, and one or more genes from the second table that are not in the first. In embodiments, the target molecules that are measured are from fewer than 500 genes (e.g., fewer than 400, 300, 200, 100, or 50 genes).

In embodiments, the cancer is lung cancer, and the plurality of target molecules detected above a threshold are selected from polypeptides and/or transcripts of one or more of Tables 2, 5, 12, or 18 (e.g., 2, 3, 5, or more genes). In embodiments, one or more target molecules are derived from one or more genes selected from each of Tables 2, 5, 12, and 18 (e.g., 2, 3, 5, or more genes). In embodiments, selection of genes from first and second tables comprises selecting one or more genes in both of the first and second tables. In embodiments, selection of genes from first and second tables comprises selecting one or more genes from the first table that are not in the second, and one or more genes from the second table that are not in the first. In embodiments, the target molecules that are measured are from fewer than 500 genes (e.g., fewer than 400, 300, 200, 100, or 50 genes). In some embodiments, the cancer is lung cancer, and the plurality of target molecules detected above a threshold are selected from polypeptides of one or more of WFDC2, CXCL17, MMP12, GDF15, CEACAM5, PRSS8, TFF1, CWC15, ALPP, GP2, INSL4, CHGA, GFRA1, AGR2, SPON1, DXO, AIF1, FKBPL, SFTPA2, or FOLR1.

In embodiments, the cancer is breast cancer, and the plurality of target molecules detected above a threshold are selected from polypeptides and/or transcripts of genes in one or more of Tables 3, 4, 6, 13, or 19 (e.g., 2, 3, 5, or more genes). In embodiments, one or more target molecules are derived from one or more genes selected from each of Tables 3, 4, 6, 13, and 19 (e.g., 2, 3, 5, or more genes). In embodiments, selection of genes from first and second tables comprises selecting one or more genes in both of the first and second tables. In embodiments, selection of genes from first and second tables comprises selecting one or more genes from the first table that are not in the second, and one or more genes from the second table that are not in the first. In embodiments, the target molecules that are measured are from fewer than 500 genes (e.g., fewer than 400, 300, 200, 100, or 50 genes). In some embodiments, the plurality of target molecules detected above a threshold are selected from polypeptides of one or more of ADAMTS15, LEP, ERBB2, ERBB4, CGA, AFP, F7, BPIFB2, SFRP1, FGFBP1, LAMA4, GP2, MIA, FGFR2, or VTCN1.

In embodiments, one or more target molecules are derived from one or more genes selected from Table 11 (e.g., 2, 3, 5, or more genes) in combination with (a) one or more genes selected from Table 5 or Table 6 (e.g., 2, 3, 5, or more genes), and/or (b) one or more genes selected from Table 7 (e.g., 2, 3, 5, or more genes). In embodiments, selection of genes from first and second tables comprises selecting one or more genes in both of the first and second tables. In embodiments, selection of genes from first and second tables comprises selecting one or more genes from the first table that are not in the second, and one or more genes from the second table that are not in the first. In embodiments, the target molecules that are measured are from fewer than 500 genes (e.g., fewer than 400, 300, 200, 100, or 50 genes).

In embodiments, one or more target molecules are derived from one or more genes selected from Table 12 (e.g., 2, 3, 5, or more genes) in combination with (a) one or more genes selected from Table 5 (e.g., 2, 3, 5, or more genes), and/or (b) one or more genes selected from Table 7 (e.g., 2, 3, 5, or more genes). In embodiments, selection of genes from first and second tables comprises selecting one or more genes in both of the first and second tables. In embodiments, selection of genes from first and second tables comprises selecting one or more genes from the first table that are not in the second, and one or more genes from the second table that are not in the first. In embodiments, the target molecules that are measured are from fewer than 500 genes (e.g., fewer than 400, 300, 200, 100, or 50 genes).

In embodiments, one or more target molecules are derived from one or more genes selected from Table 13 (e.g., 2, 3, 5, or more genes) in combination with (a) one or more genes selected from Table 4 (e.g., 2, 3, 5 or more genes), (b) one or more genes selected from Table 6 (e.g., 2, 3, 5, or more genes), and/or (c) one or more genes selected from Table 7 (e.g., 2, 3, 5, or more genes). In embodiments, selection of genes from first and second tables comprises selecting one or more genes in both of the first and second tables. In embodiments, selection of genes from first and second tables comprises selecting one or more genes from the first table that are not in the second, and one or more genes from the second table that are not in the first. In embodiments, the target molecules that are measured are from fewer than 500 genes (e.g., fewer than 400, 300, 200, 100, or 50 genes).

In embodiments, one or more target molecules are derived from one or more genes selected from Table 4 (e.g., 2, 3, 5, or more genes) in combination with (a) one or more genes selected from Table 3 (e.g., 2, 3, 5, or more genes), (b) one or more genes selected from Table 6 (e.g., 2, 3, 5, or more genes, and/or (c) one or more genes selected from Table 7 (e.g., 2, 3, 5, or more genes). In embodiments, selection of genes from first and second tables comprises selecting one or more genes in both of the first and second tables. In embodiments, selection of genes from first and second tables comprises selecting one or more genes from the first table that are not in the second, and one or more genes from the second table that are not in the first. In embodiments, the target molecules that are measured are from fewer than 500 genes (e.g., fewer than 400, 300, 200, 100, or 50 genes).

In some embodiments, one or more target molecules are derived from one or more genes selected from the genes listed in Table 8. In embodiments, the one or more target molecules are derived from at least 2, 3, 4, 5, 10, 15, 20, or 30 genes from Table 8. In embodiments, the one or more target molecules are derived from at least 5 genes from Table 8 (e.g., the first 5 genes, CEACAM5, RHOV, SFTA2, SCGB1D2, and IGF2BP1). In embodiments, the one or more target molecules are derived from at least 10 genes from Table 8. In embodiments, the one or more target molecules are derived from at least 25 genes from Table 8. In embodiments, the one or more target molecules are derived from all of the genes from Table 8. In embodiments, the target molecules that are measured are from fewer than 500 genes (e.g., fewer than 400, 300, 200, 100, or 50 genes).

In embodiments, the plurality of target molecules detected above a threshold are molecules derived from a plurality of genes selected from the group consisting of: CEACAM5, RHOV, SFTA2, SCGB1D2, IGF2BP1, SFTPA1, CA12, SFTPB, CDH3, MUC6, SLC6A14, HOXC9, AGR3, TMEM125, TFAP2B, IRX2, POTEKP, ARHGEF38, GPR87, LMX1B, ATP10B, NELL1, MUC21, SOX9, LINC00993, STMND1, ERVH48-1, SCTR, MAGEA3, MB, LEMD1, SIX4, and NXNL2. Table 8 below provides examples of highly informative cancer biomarkers.

TABLE 8

| | | | | |
|---|---|---|---|---|
| CEACAM5 | RHOV | SFTA2 | SCGB1D2 | IGF2BP1 |
| SFTPA1 | CA12 | SFTPB | CDH3 | MUC6 |
| SLC6A14 | HOXC9 | AGR3 | TMEM125 | TFAP2B |
| IRX2 | POTEKP | ARHGEF38 | GPR87 | LMX1B |
| ATP10B | NELL1 | MUC21 | SOX9 | LINC00993 |
| STMND1 | ERVH48-1 | SCTR | MAGEA3 | MB |
| LEMD1 | SIX4 | NXNL2 | | |

In some embodiments, one or more target molecules are derived from one or more genes selected from the genes listed in Table 16A or 16B. In embodiments, the one or more target molecules includes molecules derived from at least 2, 3, 4, 5, 10, 25, 50, or 60 genes from Table 16A or 16B. In embodiments, the one or more target molecules are derived from at least 5 genes from Table 16A or 16B. In embodiments, the one or more target molecules are derived from at least 25 genes from Table 16A or 16B. In embodiments, the one or more target molecules are derived from at least 50 genes from Table 16A or 16B. In embodiments, the one or more target molecules are derived from all of the genes from Table 16A or 16B. In embodiments, the target molecules that are measured are from fewer than 500 genes (e.g., fewer than 400, 300, 200, 100, or 50 genes).

TABLE 16A

ADAMTS15
ADAMTS8
AFP
AGER
AGR2
AGR3
AIF1
ALPP
ART3
CA12
CALCA
CD99
CEACAM5
CGA
CLSTN2
CXCL17
EGFR
ERBB2
ERBB4
F7
FGFBP1
FGFR2
FOLR1
GDF15
GFRA1
GP2
IL20
ITGB6
KLK8
KRT14
LAMA4
LEP
LILRB1
LY6D
MAPT
MB
MET
MIA
MILR1
MMP10
MMP12
MMP7
NELL1
OBP2B
ODAM
OSCAR
PAEP
PLA2G1B
PRSS8
RET
RGMA
SCGB1A1
SERPINA11
SERPINB5
SEZ6L2
SFRP1
SFTPA1
SFTPA2
SLITRK6
SPON1
STC2
TFF1
VTCN1
WFDC2

TABLE 16B

ADAMTS15
ADAMTS8
ADIPOQ
AFP
AGER
AGR2
AGR3
AIF1
AKR1B10
ALPP
ART3
BPIFA2
BPIFB2
CA12
CALCA
CD99
CEACAM5
CGA
CHGA
CLCA2
CLIC1
CLSTN2
CSF2
CST1
CTSE
CWC15
CXCL17
DXO
EGFR
ERBB2
ERBB4
F7
FGFBP1
FGFR2
FGFR4
FKBPL
FOLR1
GDF15
GFRA1
GP2
IL20
INSL4
ITGB6
KLK8
KRT14
LAMA4
LEP
LILRB1
LY6D
MAPT
MB
MET
MIA
MILR1
MMP10
MMP12
MMP7
MSLNL
MUCL1
MUCL3
NELL1
OBP2B
ODAM
OSCAR
PAEP
PFDN6
PLA2G1B
PRND
PRSS8
PTPRZ1
RET
RGMA
SCGB1A1
SERPINA11
SERPINB5
SEZ6L2
SFRP1
SFTPA1
SFTPA2
SHH
SLC44A4
SLITRK6
SOX2
SPINK8
SPON1
STC2
TFF1
VTCN1
WFDC2

In some embodiments, one or more target molecules are derived from one or more genes selected from the genes listed in Table 17. In embodiments, the one or more target molecules are derived from at least 2, 3, 4, 5, 10, 25, or 50 genes from Table 17. In embodiments, the one or more target molecules are derived from at least 5 genes from Table 17. In embodiments, the one or more target molecules are derived from at least 25 genes from Table 17. In embodiments, the one or more target molecules are derived from at least 50 genes from Table 17. In embodiments, the one or more target molecules are derived from all of the genes from Table 17. In embodiments, the target molecules that are measured are from fewer than 500 genes (e.g., fewer than 400, 300, 200, 100, or 50 genes).

TABLE 17

ADAMTS15
ADAMTS8
AFP
AGER
AGR3
AIF1
ALPP
ART3
CA12
CALCA
CD99
CEACAM5
CGA
CLSTN2
EGFR
ERBB2
ERBB4
F7
FGFBP1
FGFR2
FOLR1
GDF15
GFRA1
GP2
IL20
ITGB6
KLK8
KRT14
LAMA4
LEP
LILRB1
LY6D
MAPT
MB
MET
MIA
MILR1
MMP10
MMP12
NELL1
OBP2B
ODAM
OSCAR
PAEP
PLA2G1B
PRSS8
RET
RGMA
SCGB1A1
SERPINA11
SEZ6L2
SFRP1
SFTPA1
SLITRK6
SPON1
STC2

In embodiments, the one or more target molecules comprises target polypeptides, and the detecting comprises a polypeptide detection assay.

In embodiments, detecting one or more of the target molecules above a threshold level comprises (i) detection, (ii) detection above background, or (iii) detection at a level that is greater than a level of the target molecules in subjects that do not have the condition. In embodiments, detecting above a threshold comprises detection. In embodiments, detecting above a threshold comprises detection above a threshold. In embodiments, detecting above a threshold comprises detection at a level that is greater than a level of the target molecules in subjects that do not have the condition.

In embodiments, detecting one or more of the target molecules above a threshold level comprises detecting the one or more target molecules at a level that is at least about or exactly 10 times greater than a level in subjects that do not have the condition (e.g., 15, 20, 50, 100, or more times greater). In embodiments, detection above a threshold comprises detecting the one or more target molecules at a level that is at least about or exactly 25 times greater than a level in subjects that do not have the condition. In embodiments, detection above a threshold comprising detecting the one or more target molecules at a level that is at least about or exactly 50 times greater than a level in subjects that do not have the condition.

In embodiments, the one or more target molecules comprises target polynucleotides (e.g. cfRNA), and detecting the one or more of the target cfRNA molecules above a threshold level comprises detection above a threshold value of 0.5 to 5 reads per million (RPM), such as about 1, 1.5, 2, 2.5, 3, 3.5, 4, or about 4.5 RPM. In embodiments, detecting above a threshold comprises detection above 1 RPM. In embodiments, detecting above a threshold comprises detection above 1 RPM. In embodiments, detecting above a threshold comprises detection above 2 RPM. In embodiments, detecting above a threshold comprises detection above 5 RPM.

### Diseases and Disorders

Methods in accordance with embodiments of the disclosure can be used for detecting the presence or absence of any of a variety of diseases or conditions, including, but not limited to, cardiovascular disease, liver disease, or cancer. In some embodiments, the methods involve determining a cancer stage. In some embodiments, the cancer stage is stage I cancer, stage II cancer, stage III cancer, or stage IV cancer.

In some embodiments, the methods involve detecting the presence or absence of, determining the stage of, monitoring the progression of, and/or classifying a carcinoma, a sarcoma, a myeloma, a leukemia, a lymphoma, a blastoma, a germ cell tumor, or any combination thereof. In some embodiments, the carcinoma may be an adenocarcinoma. In other embodiments, the carcinoma may be a squamous cell carcinoma. In still other embodiments, the carcinoma is selected from the group consisting of small cell lung cancer, non-small-cell lung, nasopharyngeal, colorectal, anal, liver, urinary bladder, cervical, testicular, ovarian, gastric, esophageal, head-and-neck, pancreatic, prostate, renal, thyroid, melanoma, and breast carcinoma. In some embodiments, the breast carcinoma is hormone receptor negative breast carcinoma or triple negative breast carcinoma.

In some embodiments, the methods involve detecting the presence or absence of, determining the stage of, monitoring the progression of, and/or classifying a sarcoma. In embodiments, the sarcoma can be selected from the group consisting of osteosarcoma, chondrosarcoma, leiomyosarcoma, rhabdomyosarcoma, mesothelial sarcoma (mesothelioma), fibrosarcoma, angiosarcoma, liposarcoma, glioma, and astrocytoma. In still other embodiments, the methods involve detecting the presence or absence of, determining the stage of, monitoring the progression of, and/or classifying leukemia. In various embodiments, the leukemia can be selected from the group consisting of: myelogenous, granulocytic, lymphatic, lymphocytic, and lymphoblastic leukemia. In still other embodiments, the methods involve detecting the presence or absence of, determining the stage of, monitoring the progression of, and/or classifying a lymphoma. In various embodiments, the lymphoma can be selected from the group consisting of: Hodgkin's lymphoma and Non-Hodgkin's lymphoma.

Aspects of the disclosure include methods for determining a tissue of origin of a disease, wherein the tissue of origin is selected from the group consisting of pancreatic tissue, hepatobiliary tissue, liver tissue, lung tissue, brain tissue, neuroendocrine tissue, uterus tissue, renal tissue, urothelial tissue, renal tissue, cervical tissue, breast tissue, fat, colon tissue, rectum tissue, heart tissue, skeletal muscle tissue, prostate tissue and thyroid tissue.

Aspects of the invention include methods for determining a cancer cell type, wherein the cancer cell type is selected from the group consisting of bladder cancer, breast cancer, cervical cancer, colorectal cancer, endometrial cancer, esophageal cancer, gastric cancer, head/neck cancer, hepatobiliary cancer, hematological cancer, liver cancer, lung cancer, a lymphoma, a melanoma, multiple myeloma, ovarian cancer, pancreatic cancer, prostate cancer, renal cancer, thyroid cancer, urethral cancer and uterine cancer.

In some embodiments, the same assay is applied to detect any of a plurality of cancer conditions (e.g., cancer type, and/or cancer stages disclosed herein). For example, an assay in accordance with an embodiment can be used to detect the presence (and optionally stage) of a breast cancer in a sample from first subject, and repeated to detect the presence (and optionally stage) of a lung cancer in a sample from a second subject, based on evaluating biomarkers for each condition in both samples. In embodiments, the same assay is repeated across multiple samples to identify presence of at least 5, 10, 15, 20, 25, 30, 35, 40, 45, 50, 75, 100, or more cancer conditions. In embodiments, the same assay is repeated across multiple samples to identify presence of at least 10 cancer conditions. In embodiments, the same assay is repeated across multiple samples to identify presence of at least 20 cancer conditions. In embodiments, the same assay is repeated across multiple samples to identify presence of at least 30 cancer conditions. In embodiments, the same assay is repeated across multiple samples to identify presence of at least 50 cancer conditions.

### Treating Conditions

Methods disclosed herein can be used in making therapeutic decisions, guidance and monitoring, as well as development and clinical trials of cancer therapies. In embodiments, a particular treatment is selected (and optionally administered) in response to a result obtained according to a method disclosed herein. In embodiments, methods include selecting a subject identified as having a plurality of target molecules in a biological fluid, in accordance with any of the various embodiments described herein, for receiving a particular treatment, and administering that treatment.

For example, treatment efficacy can be monitored by comparing patient target molecules (e.g., polypeptides and/or cfRNA) in samples from before, during, and after treatment with particular therapies such as molecular targeted therapies (monoclonal drugs), chemotherapeutic drugs, radiation protocols, etc. or combinations of these. In some embodiments, target molecules are monitored to see if certain cancer biomarkers increase or decrease after treatment, which can allow a physician to alter a treatment (continue, stop or change treatment, for example) in a much shorter period of time than afforded by methods of monitoring that track traditional patient symptoms. In some embodiments, a method further comprises the step of diagnosing a subject based on the target molecules detected, such as diagnosing the subject with a particular stage or type of cancer associated with a detected biomarker, or reporting a likelihood that the patient has or will develop such cancer. In embodiments, methods disclosed herein further comprise selecting a treatment based on the condition detected. In embodiments, the selected treatment is administered to the subject. Where the condition is cancer, or a particular cancer type and/or stage, an appropriate anti-cancer therapy may be selected. Non-limiting examples of anti-cancer therapies include radiation therapy, surgical resection, administration of an anti-cancer agent (e.g., an immunotherapy agent, a chemotherapy agent, or the like), or a combination of one or more of these.

### Classification Model

Aspects of the disclosure are directed to classification models. For example, a machine learning or deep learning model (e.g., a disease classifier) can be used to determine a disease state based on values of one or more features determined from one or more target molecules (e.g., polypeptides and/or cfRNA). In various embodiments, the output of the machine learning or deep learning model is a predictive score or probability of a disease state (e.g., a predictive cancer score). Therefore, the machine learning or deep learning model generates a disease state classification based on the predictive score or probability.

In some embodiments, the machine learned model includes a logistic regression classifier. In other embodiments, the machine learning or deep learning model can be one of a decision tree, an ensemble (e.g., bagging, boosting, random forest), gradient boosting machine, ion, Naïve Bayes, support vector machine, or a neural network. The disease state model includes learned weights for the features that are adjusted during training. The term weights is used generically here to represent the learned quantity associated with any given feature of a model, regardless of which particular machine learning technique is used. In some embodiments, a cancer indicator score is determined by inputting values for features derived from one or more target molecules (e.g., polypeptides, cfRNA, or sequence reads thereof) into a machine learning or deep learning model.

During training, training data is processed to generate values for features that are used to train the weights of the disease state model. As an example, training data can include cfRNA data and/or WBC RNA data obtained from training samples, as well as an output label. For example, the output label can be an indication as to whether the individual is known to have a specific disease (e.g., known to have cancer) or known to be healthy (i.e., devoid of a disease). In other embodiments, the model can be used to determine a disease type, or tissue of origin (e.g., cancer tissue of origin), or an indication of a severity of the disease (e.g., cancer stage) and generate an output label therefor. Depending on the embodiment, the disease state model receives the values for one or more of the features determined from a detection assay, and computational analyses relevant to the model to be trained. In one embodiment, the one or more features comprise a quantity of one or more target molecules (e.g., polypeptides, cfRNA, or sequence reads derived therefrom). Depending on the differences between the scores output by the model-in-training and the output labels of the training data, the weights of the predictive cancer model are optimized to enable the disease state model to make more accurate predictions. In various embodiments, a disease state model may be a non-parametric model (e.g., k-nearest neighbors) and therefore, the predictive cancer model can be trained to more accurately make predictions without having to optimize parameters.

The trained disease state model can be stored in a computer readable medium, and subsequently retrieved when needed, for example, during deployment of the model.

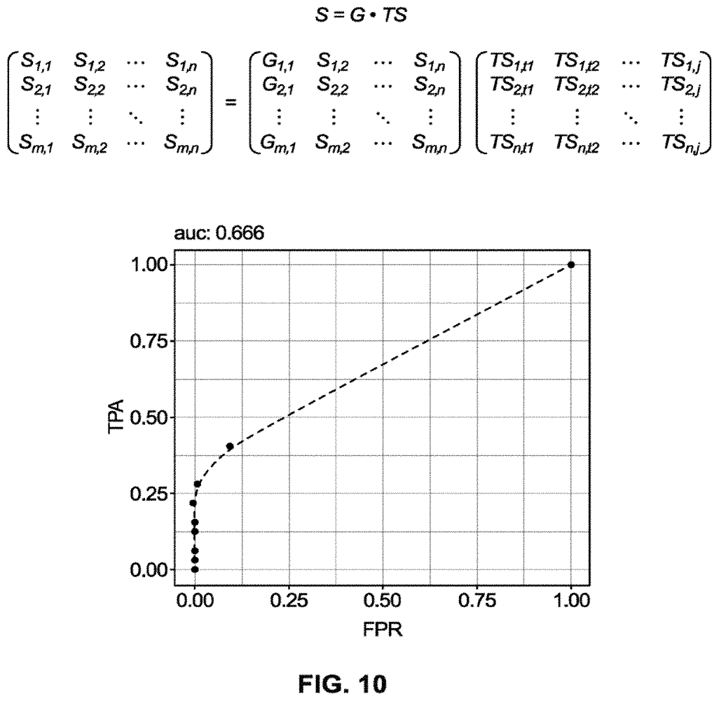

In some embodiments, the methods involve transforming a gene expression matrix (G) into a tissue score matrix (S) by multiplying the gene expression matrix (G) with a tissue specificity matrix (TS). G_{m,n} is the expression level for gene n in sample m. TS_{n,j} is the tissue specificity of gene n for tissue j. If gene n is not specific for tissue j, TS_{n,j}=0. In some embodiments, the tissue specificity matrix is calculated using the tissue RNA-seq database (GTEx). The tissue scores can be used as features to build models to classify, e.g., cancer versus non-cancer samples. In one non-limiting embodiment, the dark channel genes identified from lung cancer samples (SFTPA2, SLC39A4, NKX2_1, SFTPA1, BPIFA1, SLC34A2, CXCL17, SFTA3, MUC1, AGR2, WFDC2, ABCA12, VSIG10, CRABP2) were used to build a decision tree classifier to distinguish lung cancer from non-cancer biological fluid samples. The results of this analysis are shown in FIG. 10.

### Sequencing and Bioinformatics

Aspects of the disclosure include sequencing of nucleic acid molecules to generate a plurality of sequence reads, and bioinformatic manipulation of the sequence reads to carry out the subject methods.

In various embodiments, a sample is collected from a subject, followed by enrichment for genetic regions or genetic fragments of interest. For example, in some embodiments, a sample can be enriched by hybridization to a nucleotide array comprising cancer-related genes or gene fragments of interest. In some embodiments, a sample can be enriched for genes of interest (e.g., cancer-associated genes) using other methods known in the art, such as hybrid capture. See, e.g., Lapidus (U.S. Pat. No. 7,666,593), the contents of which is incorporated by reference herein in its entirety. In one hybrid capture method, a solution-based hybridization method is used that includes the use of biotinylated oligonucleotides and streptavidin coated magnetic beads. See, e.g., Duncavage et al., J Mol Diagn. 13(3): 325-333 (2011); and Newman et al., Nat Med. 20(5): 548-554 (2014). Isolation of nucleic acid from a sample in accordance with the methods of the disclosure can be done according to any method known in the art.

Sequencing may be by any method or combination of methods known in the art. For example, known nucleic acid sequencing techniques include, but are not limited to, classic dideoxy sequencing reactions (Sanger method) using labeled terminators or primers and gel separation in slab or capillary, sequencing by synthesis using reversibly terminated labeled nucleotides, pyrosequencing, 454 sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, Polony sequencing, and SOLiD sequencing. Sequencing of separated molecules has more recently been demonstrated by sequential or single extension reactions using polymerases or ligases as well as by single or sequential differential hybridizations with libraries of probes.

One conventional method to perform sequencing is by chain termination and gel separation, as described by Sanger et al., Proc Natl. Acad. Sci. USA, 74(12): 5463 67 (1977), the contents of which are incorporated by reference herein in their entirety. Another conventional sequencing method involves chemical degradation of nucleic acid fragments. See, Maxam et al., Proc. Natl. Acad. Sci., 74: 560 564 (1977), the contents of which are incorporated by reference herein in their entirety. Methods have also been developed based upon sequencing by hybridization. See, e.g., Harris et al., (U.S. patent application number 2009/0156412), the contents of which are incorporated by reference herein in their entirety.

A sequencing technique that can be used in the methods of the provided disclosure includes, for example, Helicos True Single Molecule Sequencing (tSMS) (Harris T. D. et al. (2008) Science 320:106-109), the contents of which are incorporated by reference herein in their entirety. Further description of tSMS is shown, for example, in Lapidus et al. (U.S. Pat. No. 7,169,560), the contents of which are incorporated by reference herein in their entirety, Lapidus et al. (U.S. patent application publication number 2009/0191565, the contents of which are incorporated by reference herein in their entirety), Quake et al. (U.S. Pat. No. 6,818,395, the contents of which are incorporated by reference herein in their entirety), Harris (U.S. Pat. No. 7,282,337, the contents of which are incorporated by reference herein in their entirety), Quake et al. (U.S. patent application publication number 2002/0164629, the contents of which are incorporated by reference herein in their entirety), and Braslavsky, et al., PNAS (USA), 100: 3960-3964 (2003), the contents of which are incorporated by reference herein in their entirety.

Another example of a nucleic acid sequencing technique that can be used in the methods of the provided disclosure is 454 sequencing (Roche) (Margulies, M et al. 2005, Nature, 437, 376-380, the contents of which are incorporated by reference herein in their entirety). Another example of a DNA sequencing technique that can be used in the methods of the provided disclosure is SOLiD technology (Applied Biosystems). Another example of a DNA sequencing technique that can be used in the methods of the provided disclosure is Ion Torrent sequencing (U.S. patent application publication numbers 2009/0026082, 2009/0127589, 2010/0035252, 2010/0137143, 2010/0188073, 2010/0197507, 2010/0282617, 2010/0300559, 2010/0300895, 2010/0301398, and 2010/0304982, the contents of each of which are incorporated by reference herein in their entirety).

In some embodiments, the sequencing technology is Illumina sequencing. Illumina sequencing is based on the amplification of DNA on a solid surface using fold-back PCR and anchored primers. Genomic DNA can be fragmented, or in the case of cfDNA, fragmentation is not needed due to the already short fragments. Adapters are ligated to the 5′ and 3′ ends of the fragments. DNA fragments that are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured and the identity of the first base is recorded. The 3′ terminators and fluorophores from each incorporated base are removed and the incorporation, detection and identification steps are repeated.

Another example of a sequencing technology that can be used in the methods of the provided disclosure includes the single molecule, real-time (SMRT) technology of Pacific Biosciences. Yet another example of a sequencing technique that can be used in the methods of the provided disclosure is nanopore sequencing (Soni G V and Meller A. (2007) Clin Chem 53: 1996-2001, the contents of which are incorporated by reference herein in their entirety). Another example of a sequencing technique that can be used in the methods of the provided disclosure involves using a chemical-sensitive field effect transistor (chemFET) array to sequence DNA (for example, as described in US Patent Application Publication No. 20090026082, the contents of which are incorporated by reference herein in their entirety). Another example of a sequencing technique that can be used in the methods of the provided disclosure involves using an electron microscope (Moudrianakis E. N. and Beer M. Proc Natl Acad Sci USA. 1965 March; 53:564-71, the contents of which are incorporated by reference herein in their entirety).

If the nucleic acid from the sample is degraded or only a minimal amount of nucleic acid can be obtained from the sample, PCR can be performed on the nucleic acid in order to obtain a sufficient amount of nucleic acid for sequencing (See, e.g., Mullis et al. U.S. Pat. No. 4,683,195, the contents of which are incorporated by reference herein in its entirety)

### Detecting Target Polypeptides

A variety of suitable methods for detecting one or more target polypeptides are available. Non-limiting examples include competitive and non-competitive immunoassays, enzyme immunoassays (EIA), radioimmunoassays (RIA), antigen capture assays, two-antibody sandwich assays, Western blot analysis, enzyme linked immunosorbant assays (ELISA), colorimetric assays, chemiluminescent assays, fluorescence assays, immunohistochemistry assays, chromatography, liquid chromatography, size exclusion chromatography, high performance liquid chromatography (HPLC), gas chromatography, mass spectrometry, tandem mass spectrometry, matrix assisted laser desorption/ionization-time of flight (MALDI-TOF) mass spectrometry, electrospray ionization (ESI) mass spectrometry, surface-enhanced laser desorption/ionization-time of flight (SELDI-TOF) mass spectrometry, quadrupole-time of flight (Q-TOF) mass spectrometry, atmospheric pressure photoionization mass spectrometry (APPI-MS), Fourier transform mass spectrometry (FTMS), matrix-assisted laser desorption/ionization—Fourier transform-ion cyclotron resonance (MALDI-FT-ICR) mass spectrometry, secondary ion mass spectrometry (SIMS), microscopy, microfluidic chip-based assays, and surface plasmon resonance.

In some embodiments, one or more polypeptides are detected (and optionally, relative level determined) using a proximity extension assay (PEA). In embodiments, PEA comprises the simultaneous binding of a pair of proximity probes to a biomarker in proximity. Upon binding of the pair of proximity probes to the biomarker, the nucleic acid domains are capable of interacting and forming a nucleic acid duplex, which may enable at least one of the nucleic acid domains to be extended from its 3′ end. This extension product forms a detectable nucleic acid detection product, optionally following amplification e.g. by PCR. Exemplary PEA methods are described in greater detail in WO 2012/104261 and US2015/0044674, which are incorporated herein by reference. Target polypeptides may be detected singly, or more preferably multiple target polypeptides may be detected simultaneously in a multiplexed detection format.

In some embodiments, one or more polypeptides are detected (and optionally, relative level determined) using a Multiple Reaction Monitoring (MRM) assay. A variety of MRM methods are available. In embodiments, the MRM assay uses triple quadrupole mass spectrometers coupled to liquid chromatography to detect or quantify target polypeptides. In the first quadrupole (Q1), a peptide that corresponds to a protein of interest is selected. The peptide is then fragmented in the second quadrupole (Q2) and a filter is applied to allow a particular fragment to enter into the third quadrupole (Q3) where its intensity is measured. Target polypeptides may be detected singly, or more preferably multiple target polypeptides may be detected simultaneously in a multiplexed detection format. Further non-limiting examples of MRM are described in US20190277846 and US20180024108, which are incorporated herein by reference.

In some embodiments, one or more polypeptides are detected (and optionally, relative level determined) using a quantitation platform integrating nanoparticle (NP) protein coronas with liquid chromatography-mass spectrometry. In embodiments, the platform is a Proteograph platform. In embodiments, a protein corona is a protein layer adsorbed onto NPs upon contact with biological fluids. Varying the physicochemical properties of engineered NPs translates to distinct protein corona patterns enabling differential and reproducible interrogation of biological samples. In embodiments, the Proteograph platform uses a multi-NP protein corona approach and mass spectrometry. In embodiments, this approach includes four steps: (1) NP-biological sample incubation and protein corona formation; (2) NP protein corona purification by a magnet; (3) digestion of corona proteins; and (4) LC-MS/MS analysis. In this context, each biological sample-NP well is a sample, for a total of 96 samples per plate. Target polypeptides may be detected singly, or more preferably multiple target polypeptides may be detected simultaneously in a multiplexed detection format. A non-limiting example of an NP-based protein corona detection is described in WO2020096631A2, which is incorporated herein by reference.

### Computer Systems and Devices

Aspects of the disclosure described herein can be performed using any type of computing device, such as a computer, that includes a processor, e.g., a central processing unit, or any combination of computing devices where each device performs at least part of the process or method. In some embodiments, systems and methods described herein may be performed with a handheld device, e.g., a smart tablet, or a smart phone, or a specialty device produced for the system.

Methods of the disclosure can be performed using software, hardware, firmware, hardwiring, or combinations of any of these. Features implementing functions can also be physically located at various positions, including being distributed such that portions of functions are implemented at different physical locations (e.g., imaging apparatus in one room and host workstation in another, or in separate buildings, for example, with wireless or wired connections).

Processors suitable for the execution of computer programs include, by way of example, both general and special purpose microprocessors, and any one or more processors of any kind of digital computer. Generally, a processor will receive instructions and data from a read-only memory or a random access memory, or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including, by way of example, semiconductor memory devices, (e.g., EPROM, EEPROM, solid state drive (SSD), and flash memory devices); magnetic disks, (e.g., internal hard disks or removable disks); magneto-optical disks; and optical disks (e.g., CD and DVD disks). The processor and the memory can be supplemented by, or incorporated in, special purpose logic circuitry.

To provide for interaction with a user, the subject matter described herein can be implemented on a computer having an I/O device, e.g., a CRT, LCD, LED, or projection device for displaying information to the user and an input or output device such as a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The subject matter described herein can be implemented in a computing system that includes a back-end component (e.g., a data server), a middleware component (e.g., an application server), or a front-end component (e.g., a client computer having a graphical user interface or a web browser through which a user can interact with an implementation of the subject matter described herein), or any combination of such back-end, middleware, and front-end components. The components of the system can be interconnected through a network by any form or medium of digital data communication, e.g., a communication network. For example, a reference set of data may be stored at a remote location and a computer can communicate across a network to access the reference data set for comparison purposes. In other embodiments, however, a reference data set can be stored locally within the computer, and the computer accesses the reference data set within the CPU for comparison purposes. Examples of communication networks include, but are not limited to, cell networks (e.g., 3G or 4G), a local area network (LAN), and a wide area network (WAN), e.g., the Internet.

The subject matter described herein can be implemented as one or more computer program products, such as one or more computer programs tangibly embodied in an information carrier (e.g., in a non-transitory computer-readable medium) for execution by, or to control the operation of, a data processing apparatus (e.g., a programmable processor, a computer, or multiple computers). A computer program (also known as a program, software, software application, app, macro, or code) can be written in any form of programming language, including compiled or interpreted languages (e.g., C, C++, Perl), and it can be deployed in any form, including as a stand-alone program or as a module, component, subroutine, or other unit suitable for use in a computing environment. Systems and methods of the disclosure can include instructions written in any suitable programming language known in the art, including, without limitation, C, C++, Perl, Java, ActiveX, HTML5, Visual Basic, or JavaScript.

A computer program does not necessarily correspond to a file. A program can be stored in a file or a portion of a file that holds other programs or data, in a single file dedicated to the program in question, or in multiple coordinated files (e.g., files that store one or more modules, sub-programs, or portions of code). A computer program can be deployed to be executed on one computer or on multiple computers at one site or distributed across multiple sites and interconnected by a communication network.

A file can be a digital file, for example, stored on a hard drive, SSD, CD, or other tangible, non-transitory medium. A file can be sent from one device to another over a network (e.g., as packets being sent from a server to a client, for example, through a Network Interface Card, modem, wireless card, or similar).

Writing a file according to the disclosure involves transforming a tangible, non-transitory computer-readable medium, for example, by adding, removing, or rearranging particles (e.g., with a net charge or dipole moment into patterns of magnetization by read/write heads), the patterns then representing new collocations of information about objective physical phenomena desired by, and useful to, the user. In some embodiments, writing involves a physical transformation of material in tangible, non-transitory computer readable media (e.g., with certain optical properties so that optical read/write devices can then read the new and useful collocation of information, e.g., burning a CD-ROM). In some embodiments, writing a file includes transforming a physical flash memory apparatus such as NAND flash memory device and storing information by transforming physical elements in an array of memory cells made from floating-gate transistors. Methods of writing a file are well-known in the art and, for example, can be invoked manually or automatically by a program or by a save command from software or a write command from a programming language.

Suitable computing devices typically include mass memory, at least one graphical user interface, at least one display device, and typically include communication between devices. The mass memory illustrates a type of computer-readable media, namely computer storage media. Computer storage media may include volatile, nonvolatile, removable, and non-removable media implemented in any method or technology for storage of information, such as computer readable instructions, data structures, program modules, or other data. Examples of computer storage media include RAM, ROM, EEPROM, flash memory, or other memory technology, CD-ROM, digital versatile disks (DVD) or other optical storage, magnetic cassettes, magnetic tape, magnetic disk storage or other magnetic storage devices, Radiofrequency Identification (RFID) tags or chips, or any other medium that can be used to store the desired information, and which can be accessed by a computing device.

Functions described herein can be implemented using software, hardware, firmware, hardwiring, or combinations of any of these. Any of the software can be physically located at various positions, including being distributed such that portions of the functions are implemented at different physical locations.

As one skilled in the art would recognize as necessary or best-suited for performance of the methods of the disclosure, a computer system for implementing some or all of the described inventive methods can include one or more processors (e.g., a central processing unit (CPU) a graphics processing unit (GPU), or both), main memory and static memory, which communicate with each other via a bus.

A processor will generally include a chip, such as a single core or multi-core chip, to provide a central processing unit (CPU). A process may be provided by a chip from Intel or AMD.

Memory can include one or more machine-readable devices on which is stored one or more sets of instructions (e.g., software) which, when executed by the processor(s) of any one of the disclosed computers can accomplish some or all of the methodologies or functions described herein. The software may also reside, completely or at least partially, within the main memory and/or within the processor during execution thereof by the computer system. Preferably, each computer includes a non-transitory memory such as a solid state drive, flash drive, disk drive, hard drive, etc.

While the machine-readable devices can in an exemplary embodiment be a single medium, the term “machine-readable device” should be taken to include a single medium or multiple media (e.g., a centralized or distributed database, and/or associated caches and servers) that store the one or more sets of instructions and/or data. These terms shall also be taken to include any medium or media that are capable of storing, encoding, or holding a set of instructions for execution by the machine and that cause the machine to perform any one or more of the methodologies of the present disclosure. These terms shall accordingly be taken to include, but not be limited to, one or more solid-state memories (e.g., subscriber identity module (SIM) card, secure digital card (SD card), micro SD card, or solid-state drive (SSD)), optical and magnetic media, and/or any other tangible storage medium or media.

A computer of the disclosure will generally include one or more I/O device such as, for example, one or more of a video display unit (e.g., a liquid crystal display (LCD) or a cathode ray tube (CRT)), an alphanumeric input device (e.g., a keyboard), a cursor control device (e.g., a mouse), a disk drive unit, a signal generation device (e.g., a speaker), a touchscreen, an accelerometer, a microphone, a cellular radio frequency antenna, and a network interface device, which can be, for example, a network interface card (NIC), Wi-Fi card, or cellular modem.

Any of the software can be physically located at various positions, including being distributed such that portions of the functions are implemented at different physical locations.

Additionally, systems of the disclosure can be provided to include reference data. Any suitable genomic data may be stored for use within the system. Examples include, but are not limited to: comprehensive, multi-dimensional maps of the key genomic changes in major types and subtypes of cancer from The Cancer Genome Atlas (TCGA); a catalog of genomic abnormalities from The International Cancer Genome Consortium (ICGC); a catalog of somatic mutations in cancer from COSMIC; the latest builds of the human genome and other popular model organisms; up-to-date reference SNPs from dbSNP; gold standard indels from the 1000 Genomes Project and the Broad Institute; exome capture kit annotations from Illumina, Agilent, Nimblegen, and Ion Torrent; transcript annotations; small test data for experimenting with pipelines (e.g., for new users).

In some embodiments, data is made available within the context of a database included in a system. Any suitable database structure may be used including relational databases, object-oriented databases, and others. In some embodiments, reference data is stored in a relational database such as a “not-only SQL” (NoSQL) database. In various embodiments, a graph database is included within systems of the disclosure. It is also to be understood that the term “database” as used herein is not limited to one single database; rather, multiple databases can be included in a system. For example, a database can include two, three, four, five, six, seven, eight, nine, ten, fifteen, twenty, or more individual databases, including any integer of databases therein, in accordance with embodiments of the disclosure. For example, one database can contain public reference data, a second database can contain test data from a patient, a third database can contain data from healthy subjects, and a fourth database can contain data from sick subjects with a known condition or disorder. It is to be understood that any other configuration of databases with respect to the data contained therein is also contemplated by the methods described herein.

### Illustrative Embodiments

The present disclosure provides the following illustrative embodiments.

Embodiment 1. A method of detecting cancer in a subject, the method comprising:
(a) measuring a plurality of target molecules in a biological fluid of the subject, wherein the plurality of target molecules are selected from polypeptides of Table 11; and
(b) detecting the cancer, wherein detecting the cancer comprises detecting one or more of the target molecules above a threshold level.

Embodiment 2. The method of embodiment 1, wherein the plurality of target molecules are selected from polypeptides of one or more of Tables 8 or 12-19.

Embodiment 3. The method of embodiment 1, wherein the plurality of target molecules are selected from at least 5, 10, 15, or 20 polypeptides of Tables 8, 11-14 or 17-19.

Embodiment 4. The method of any one of embodiments 1-3, wherein the plurality of target molecules comprises a plurality of polypeptides from (i) Table 11; (ii) each of Tables 2, 5, and 12, (iii) each of Tables 3, 4, 6, and 13, (iv) Table 14, (v) Table 8, or (v) Tables 18 and 19.

Embodiment 5. The method of any one of embodiments 1-4, wherein the plurality of target molecules comprise at least 30 polypeptides of one or more of Tables 11-15.

Embodiment 6. The method of any one of embodiments 1-5, wherein the plurality of target molecules are selected from polypeptides of Table 14.

Embodiment 7. The method of any one of embodiments 1-5, wherein the plurality of target molecules detected above a threshold are polypeptides selected from the group consisting of: ADAMTS15, AFP, AGR2, AIF1, ALPP, BPIFB2, CEACAM5, CGA, CHGA, CWC15, CXCL17, DXO, ERBB2, ERBB4, F7, FGFBP1, FGFR2, FKBPL, FOLR1, GDF15, GFRA1, GP2, INSL4, LAMA4, LEP, MIA, MMP12, PRSS8, SFRP1, SFTPA2, SPON1, TFF1, VTCN1, and WFDC2.

Embodiment 8. The method of any one of embodiments 1-5, wherein the plurality of target molecules detected above a threshold are selected from polypeptides of Table 8.

Embodiment 9. The method of any one of embodiments 1-5, wherein the plurality of target molecules detected above a threshold are polypeptides selected from the group consisting of: CEACAM5, RHOV, SFTA2, SCGB1D2, IGF2BP1, SFTPA1, CA12, SFTPB, CDH3, MUC6, SLC6A14, HOXC9, AGR3, TMEM125, TFAP2B, IRX2, POTEKP, ARHGEF38, GPR87, LMX1B, ATP10B, NELL1, MUC21, SOX9, LINC00993, STMND1, ERVH48-1, SCTR, MAGEA3, MB, LEMD1, SIX4, and NXNL2.

Embodiment 10. The method of any one of embodiments 1-9, wherein the plurality of target molecules comprise (a) polypeptides of one or more of Tables 11-14, and (b) one or more polypeptides of Tables 1-6.

Embodiment 11. The method of any one of embodiments 1-10, wherein the plurality of target molecules comprises (a) polypeptides of one or more of Tables 8 or 11-14, and (b) one or more polypeptides of Table 7.

Embodiment 12. The method of any one of embodiments 1-5, wherein (i) the cancer is lung cancer, and (ii) the plurality of target molecules detected above a threshold are selected from polypeptides of Table 18.

Embodiment 13. The method of any one of embodiments 1-5, wherein (i) the cancer is lung cancer, and (ii) the plurality of target molecules detected above a threshold are selected from polypeptides of one or more of WFDC2, CXCL17, MMP12, GDF15, or CEACAM5.

Embodiment 14. The method of any one of embodiments 1-5, wherein (i) the cancer is breast cancer, and (ii) the plurality of target molecules detected above a threshold are selected from polypeptides of Table 19.

Embodiment 15. The method of embodiment 14, wherein the plurality of target molecules detected above a threshold are selected from polypeptides of one or more of ADAMTS15, LEP, ERBB2, ERBB4, or CGA.

Embodiment 16. The method of any one of embodiments 1-5, wherein the plurality of target molecules comprises polypeptides of Table 16A or Table 16B.

Embodiment 17. The method of embodiment 16, wherein the plurality of target molecules comprises polypeptides of Table 17.

Embodiment 18. The method of embodiment 16, wherein the plurality of target molecules comprises polypeptides selected from AGR3, CA12, CEACAM5, CXCL17, GP2, IL20, MMP7, TFF1, VTCN1.

Embodiment 19. The method of any one of embodiments 1-18, wherein:
(a) the plurality of target molecules further comprises cell-free polynucleotides comprising (i) cell-free DNA (cfDNA) from genes encoding the polypeptides, and/or (ii) cell-free RNA (cfRNA) transcripts of the genes encoding the polypeptides; and
(b) detecting one or more of the target molecules above a threshold level comprises (i) detecting one or more of the polypeptides above a first threshold level, and (ii) for each of the polypeptides detected above the first threshold level, detecting a corresponding cell-free polynucleotide above a second threshold level.

Embodiment 20. The method of embodiment 19, wherein the cell-free polynucleotides comprise cfRNA.

Embodiment 21. The method of embodiment 19, wherein the cell-free polynucleotides comprise cfDNA.

Embodiment 22. The method of embodiment 21, wherein the cfDNA is methylated cfDNA.

Embodiment 23. The method of any one of embodiments 1-22, wherein the measuring comprises sequencing, microarray analysis, reverse transcription PCR, real-time PCR, quantitative real-time PCR, digital PCR, digital droplet PCR, digital emulsion PCR, multiplex PCR, hybrid capture, oligonucleotide ligation assays, or any combination thereof.

Embodiment 24. The method of any one of embodiments 19-23, wherein the measuring comprises sequencing the cell-free polynucleotides to produce sequence reads.

Embodiment 25. The method of embodiment 24, wherein the sequencing comprises whole transcriptome sequencing.

Embodiment 26. The method of embodiment 24 or 25, wherein the sequencing comprises sequencing cDNA molecules reverse transcribed from the cfRNA.

Embodiment 27. The method of embodiment 24, wherein the sequencing comprises sequencing an enriched population of cfRNA or cfDNA.

Embodiment 28. The method of any one of embodiments 1-27, wherein the biological fluid comprises blood, plasma, serum, urine, saliva, pleural fluid, pericardial fluid, cerebrospinal fluid (CSF), peritoneal fluid, or any combination thereof.

Embodiment 29. The method of embodiment 28, wherein the biological fluid comprises blood, a blood fraction, plasma, or serum of the subject.

Embodiment 30. The method of any one of embodiments 1-29, wherein detecting one or more of the target molecules above a threshold level comprises (i) detection, (ii) detection above background, or (iii) detection at a level that is greater than a level of the one or more target molecules in subjects that do not have the cancer.

Embodiment 31. The method of any one of embodiments 1-29, wherein detecting one or more of the target molecules above a threshold level comprises detecting the one or more target molecules at a level that is at least about 10 times greater than a level in subjects that do not have the cancer.

Embodiment 32. The method of any one of embodiments 24-29, wherein detecting one or more of the cell-free polynucleotides above a threshold level comprises detection above a threshold value of 0.5 to 5 reads per million (RPM).

Embodiment 33. The method of any one of embodiments 19-29, wherein the cell-free polynucleotides comprise cfRNA transcripts, and detecting one or more of the cfRNA transcripts above the second threshold level comprises:
(a) determining an indicator score for cfRNA transcript by comparing the expression level of each of the cfRNA transcript to an RNA tissue score matrix;
(b) aggregating the indicator scores for each cfRNA transcript; and,
(c) detecting the cancer when the indicator score exceeds a threshold value.

Embodiment 34. The method of any one of embodiments 24-33, wherein detecting one or more of the cell-free polynucleotides above a threshold level comprises inputting the sequence reads into a machine learning or deep learning model.

Embodiment 35. The method of embodiment 34, wherein the machine learning or deep learning model comprises logistic regression, random forest, gradient boosting machine, Naïve Bayes, neural network, or multinomial regression.

Embodiment 36. The method of embodiment 34, wherein the machine learning or deep learning model transforms the values of the one or more features to the disease state prediction for the subject through a function comprising learned weights.

Embodiment 37. The method of any one of embodiments 1-36, wherein the cancer comprises:
(i) a carcinoma, a sarcoma, a myeloma, a leukemia, a lymphoma, a blastoma, a germ cell tumor, or any combination thereof,
(ii) a carcinoma selected from the group consisting of adenocarcinoma, squamous cell carcinoma, small cell lung cancer, non-small-cell lung cancer, nasopharyngeal, colorectal, anal, liver, urinary bladder, testicular, cervical, ovarian, gastric, esophageal, head-and-neck, pancreatic, prostate, renal, thyroid, melanoma, and breast carcinoma;
(iii) hormone receptor negative breast carcinoma or triple negative breast carcinoma;
(iv) a sarcoma selected from the group consisting of: osteosarcoma, chondrosarcoma, leiomyosarcoma, rhabdomyosarcoma, mesothelial sarcoma (mesothelioma), fibrosarcoma, angiosarcoma, liposarcoma, glioma, and astrocytoma;
(v) a leukemia selected from the group consisting of myelogenous, granulocytic, lymphatic, lymphocytic, and lymphoblastic leukemia; or
(vi) a lymphoma selected from the group consisting of: Hodgkin's lymphoma and Non-Hodgkin's lymphoma.

Embodiment 38. The method of any one of embodiments 1-37, wherein detecting the cancer comprises determining a cancer stage, determining cancer progression, determining a cancer type, determining cancer tissue of origin, or a combination thereof.

Embodiment 39. The method of any one of embodiments 1-38, further comprising selecting a treatment based on the cancer detected.

Embodiment 40. The method of embodiment 39, wherein the treatment comprises surgical resection, radiation therapy, or administering an anti-cancer agent.

Embodiment 41. The method of embodiment 39 or 40, wherein the method further comprises treating the subject with the selected treatment.

Embodiment 42. A computer system for implementing one or more steps in the method of any one of embodiments 1-41.

Embodiment 43. A non-transitory, computer-readable medium, having stored thereon computer-readable instructions for implementing one or more steps in the method of any one of embodiments 1-41.

### EXAMPLES

It is understood that the examples and embodiments described herein are for illustrative purposes only and that various modifications or changes in light thereof will be suggested to persons skilled in the art and are to be included within the spirit and purview of this application and scope of the appended claims.

### Example 1: Detection of Tissue-Specific RNA in the Plasma of Cancer Patients

Cell-free RNA (cfRNA) is a promising analyte for cancer detection, but a comprehensive assessment of cfRNA is lacking. To characterize tumor-derived RNA in plasma, we performed an exploratory analysis from a Circulating Cell-free Genome Atlas (CCGA) substudy to examine cfRNA expression in participants with and without cancer. This analysis focused on breast, lung, and colorectal cancers due to their high incidence in the general population and in CCGA.

We selected 210 participants from the CCGA training set (Klein et al., ASCO, 2018). A total of 98 participants were diagnosed with stage III cancer at the time of blood draw (breast (47 patients), lung (32 patients), colorectal (15 patients), and anorectal (4 patients)). Stage III samples were selected to maximize signal in the blood and avoid confounding signal from potential secondary metastases. 112 non-cancer participants frequency-age-matched to the cancer group were also included. For each participant, whole transcriptome libraries from buffy coat, cfRNA, and FFPE of tumor tissue biopsies were generated.

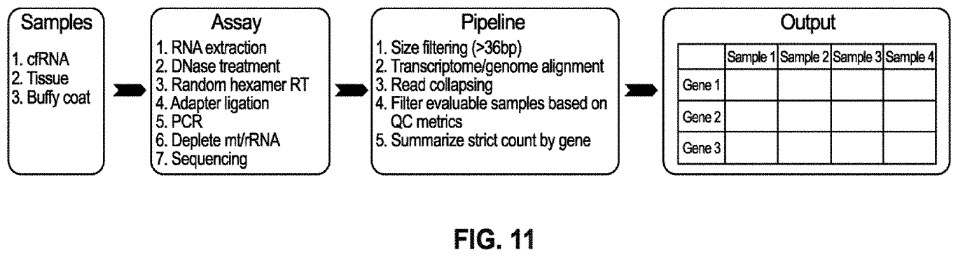

Nucleic acids were extracted from participant plasma, samples were DNAse-treated to remove cell-free DNA (cfDNA) and genomic DNA, and reverse transcription was performed using random hexamer primers to capture the whole transcriptome for each study participant. The resulting cDNA was converted into DNA libraries, amplified, and depleted of abundant sequences arising from ribosomal, mitochondrial, and blood-related transcripts, such as globins. The resulting whole-transcriptome RNA-seq libraries were sequenced at a depth of ˜750M paired-end reads per sample and analyzed using a custom bioinformatics pipeline that generated UMI-collapsed counts for each gene on a sample-by-sample basis. This same procedure was used to create and analyze RNA-seq libraries from matched buffy coat and tissue RNA when available. Due to the presence of residual DNA contamination, all downstream analyses relied on the use of strict RNA reads, defined in this example as read pairs where at least one read overlapped an exon-exon junction. FIG. 11 shows a summary of the end-to-end workflow. Table 9 provides a summary of participant samples:

TABLE 9

| Disease Status | Passed QC | cfRNA | WBC | Tissue |
|---|---|---|---|---|
| Breast | Fail | 1 | 0 | 0 |
| Lung | Fail | 2 | 1 | 0 |
| Non-cancer | Fail | 4 | 0 | 0 |
| Anorectal | Pass | 4 | 1 | 4 |
| Breast | Pass | 46 | 32 | 40 |
| Colorectal | Pass | 15 | 11 | 10 |
| Lung | Pass | 30 | 26 | 12 |
| Non-cancer | Pass | 89 | 93 | 0 |
| Young Healthy | Pass | 19 | 19 | 0 |
| Total | NA | 210 | 183 | 66 |

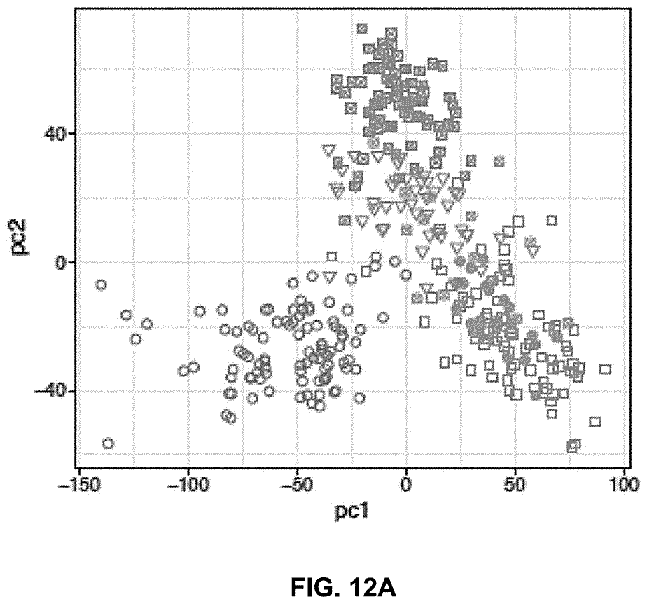

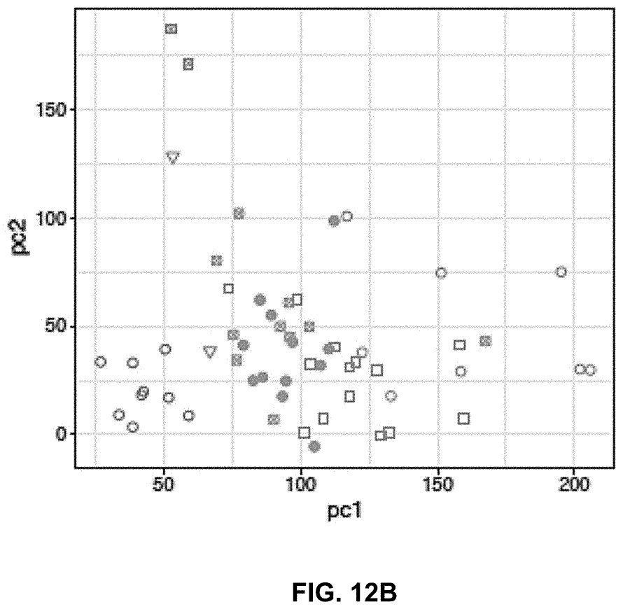

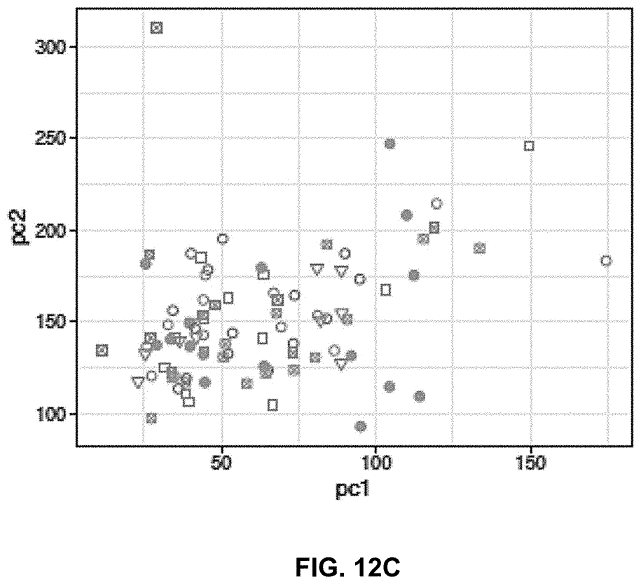

We compared our data to RNA samples from TCGA (FIG. 12A). When we projected CCGA tumor tissue RNA-seq data onto the principal components derived from TCGA tumor tissue RNA-seq data, the CCGA tumor tissue samples were separable by cancer type (FIG. 12B). These results suggest that the expression profiles of CCGA and TCGA tumors were very similar in spite of differences in sample collection/handling/library preparation, and validate the analytical approach. A projection of cancer cfRNA samples from the CCGA cohort onto the principal components derived from TCGA tumor tissue RNA-seq data showed no separation of the sample by cancer type (FIG. 12C), implying that cancer type was not the dominant source of variance in cfRNA.

The majority of cfRNA in plasma is thought to originate from healthy immune cells. As such, we treated these transcripts as background noise and focused on tumor-derived cfRNA as a source of cancer signal. Our analysis identified two classes of genes in cfRNA data: “dark channels” and “dark channel biomarkers”. Dark channels are genes that were not detected in the cfRNA of non-cancer participants. Of 57,783 annotated genes, 39,564 (68%) were identified as dark channels. Dark channel biomarker (DCB) genes met three criteria: 1) median expression of the gene in the non-cancer cohort was zero, 2) gene expression was detected in more than one participant in the cancer cohort, and 3) gene expression was up-regulated in the cancer group.

DCB genes were identified for lung cancer: SLC34A2, GABRG1, ROS1, AGR2, GNAT3, SFTPA2, MUC5B, SFTA3, SMIM22, CXCL17, BPIFA1, WFDC2, NKX2-1, and GGTLC1 (see Table 2). 10 DCB genes were identified for breast cancer: RNU1-1, CSN1S1, FABP7, OPN1SW, SCGB2A2, LALBA, CASP14, KLK5, WFDC2, and VTCN1 (see Table 3). No DCB genes were identified for colorectal cancer.

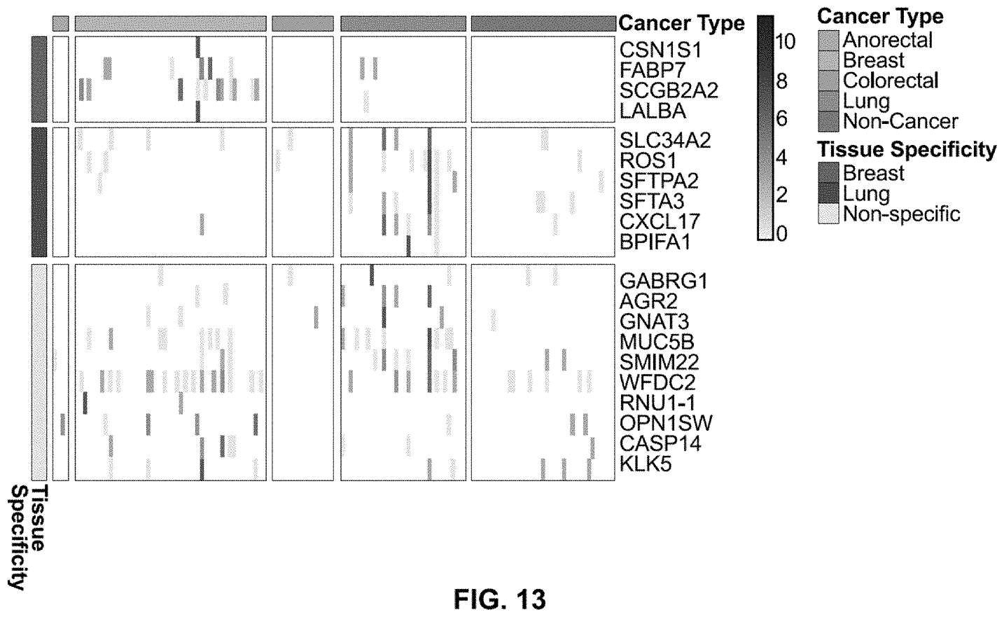

DCB genes exhibited several distinct characteristics. First, DCB genes were enriched for tissue-specific genes (FIG. 13). Among the 57,783 annotated genes, 0.3% were lung-specific and 0.2% were breast-specific. In comparison, 50% of the lung DCB genes were lung-specific, and 44% of the breast DCB genes were breast-specific (as defined by the protein atlas database (Uhlén et al., Science, 2015)).

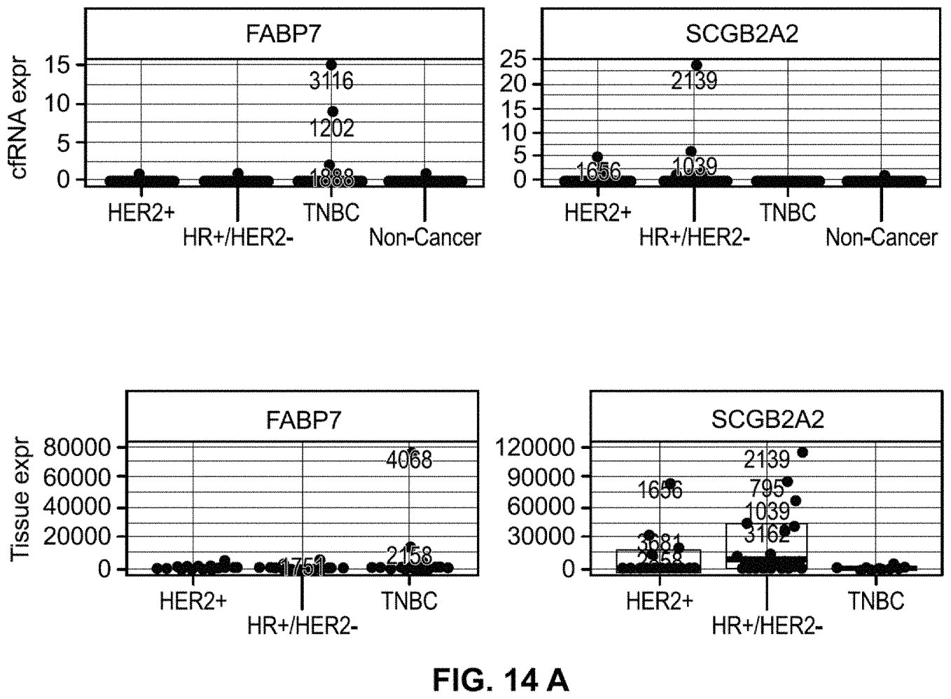

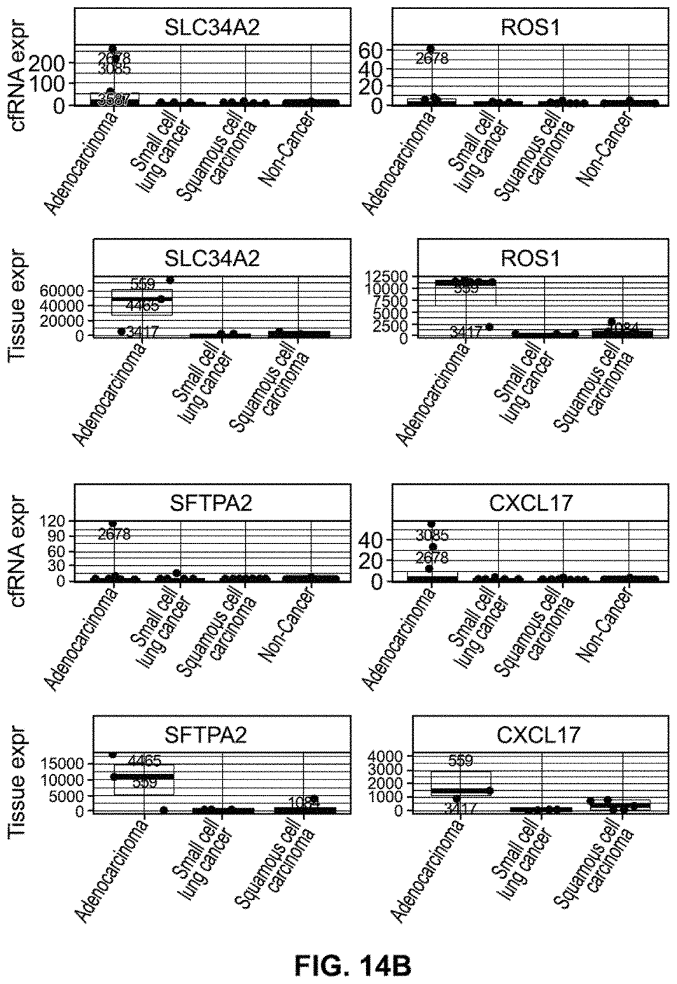

Moreover, some DCB genes were subtype-specific biomarkers that were only detected in certain cancer subtypes (FIGS. 14A and 14B). FABP7 was only detected in triple negative breast cancer (TNBC) samples. Conversely, SCGB2A2 was not detected in TNBC, but was detected in HER2+ and HR+/HER− breast cancer samples. SLC34A2, ROS1, SFTPA2 and CXCL17 genes were detected in cfRNA of lung adenocarcinoma patient samples but not in squamous cell carcinoma patient samples. These subtype-specific genes also had higher expression in tumor tissue compared to other subtypes of cancer originating from the same organ.

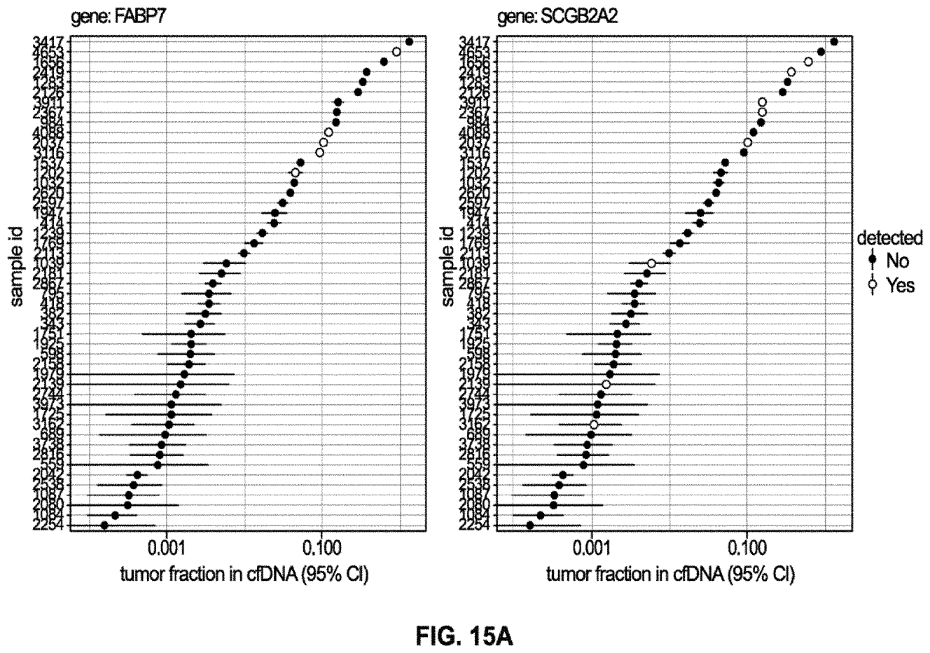

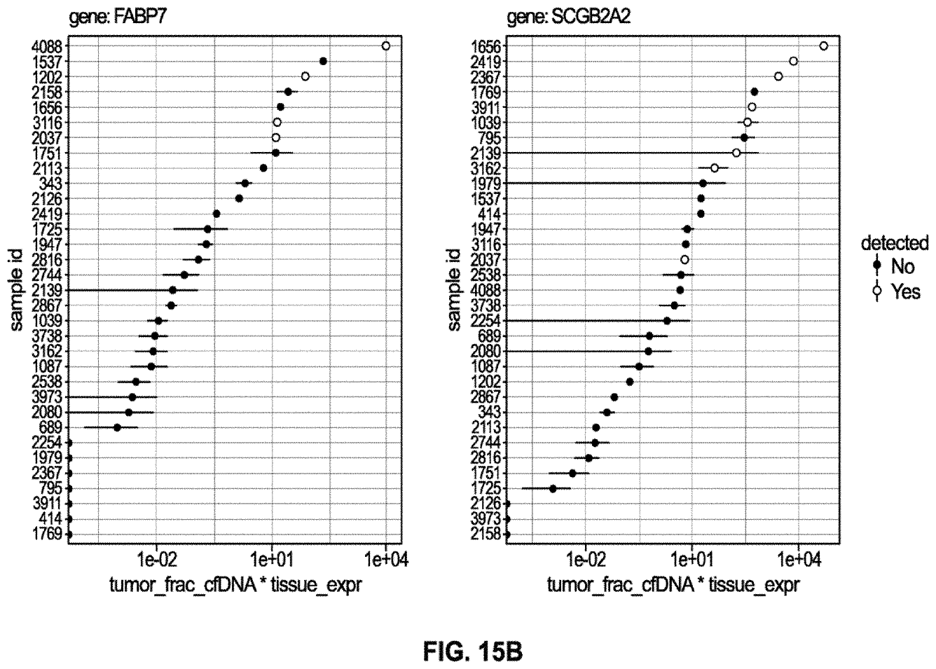

In order to determine the source of tumor-associated transcripts in the blood, concordance between cfRNA and tumor tissue RNA for dark channel biomarker genes was assessed. High concordance between cfRNA and tumor tissue expression was observed (FIG. 15A). Genes not detected in the tumor tissue were unlikely to be detected in the matched cfRNA sample, and genes detected in the tumor tissue were more likely to be detected in the matched cfRNA sample. Additionally, tumor content, measured as the product of cfDNA tumor fraction for a given patient and the gene expression in matched tumor tissue, was a strong predictor of the detectability of a DCB gene in the cfRNA of breast cancer patients (FIG. 15B).

Dark channel biomarkers (DCBs), transcripts that were not found in cfRNA from non-cancer subjects, exhibited the potential for high signal-to-noise in cancer patients. DCB signal was correlated with tumor content (measured as the product of tumor fraction in the blood and RNA expression in the tissue). cfRNA DCBs were identified in cancer participants in a tissue- and subtype-specific manner. We observed cases where high tumor tissue expression led to DCB signal amplification and enabled detection of cancer in patients with low cfDNA tumor fraction. Taken together, these data suggest that tissue-specific transcripts have potential for use in blood-based multi-cancer detection.

### Example 2: Identifying Biomarkers in Heterogeneous Samples

We observed two common sources of false-positives in biomarker discovery on heterogeneous samples using standard differential expression (DE) analysis. First, the gene expression follows bimodal distribution due to genetic heterogeneity or gene amplification drop-out in both control and cancer groups. Second, a single influential outlier inflated the slope and p-value of the generalized linear model (GLM).

A method was developed to identify differentially expressed genes in highly heterogeneous samples, such as cfRNA based on tissue expression, referred to as heteroDE. The heteroDE model uses a negative binomial generalized linear model (NB-GLM). To reduce the false-positives, heteroDE includes two additional functionalities: (1) it checks if the gene expression in the non-cancer group follows bimodal distribution due to genetic heterogeneity or gene amplification drop-out; and (2) it checks if only a single outlier sample is influencing the p-value of the NB-GLM. The outlier sample is identified using Cook's distance. The NB-GLM is performed for a second time without the sample with the largest Cook's distance.

In contrast to prior differential expression (DE) methods, heteroDE uses the tumor content as a covariate in the NG-GLM. The tumor content for the non-cancer samples was set to zero. The hypothesis for a cfRNA tumor biomarker gene was that the higher of the gene's expression in the tissue and the larger the tumor fraction in the cfDNA, the more likely it is to detect that gene in cfRNA. When we applied this method to breast cancer samples, we identified 9 cfRNA biomarkers: TRGV10, SCGB2A2, CASP14, FABP7, CRABP2, VGLL1, SERPINB5, TFF1, and AC007563.5 (see Table 4). Three of these biomarkers (FABP7, SCGB2A2, CASP14) overlap with the genes identified as DCB genes.

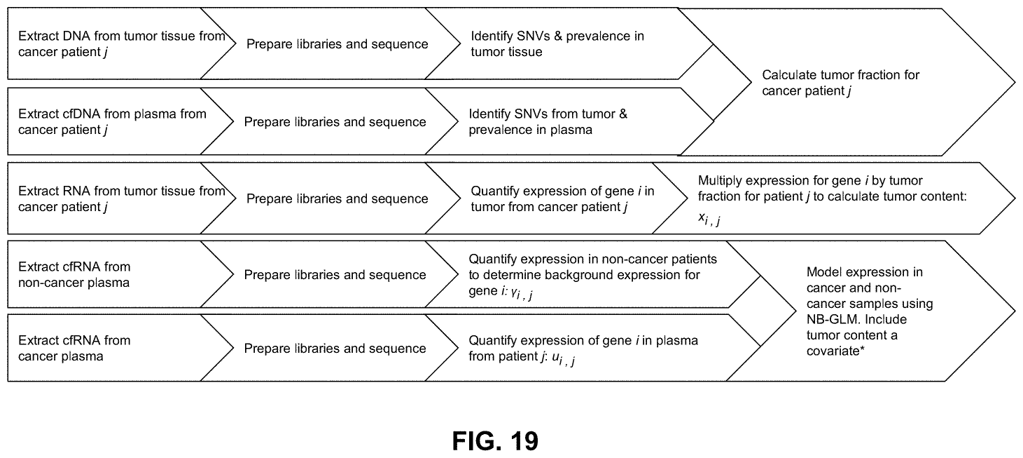

An example workflow illustrating the sample processing and parameter determination in accordance with heteroDE is shown in FIG. 19. Tumor content was constrained to zero for non-cancer subjects, due to a lack of tissue sample. An example implementation of the workflow is given by:
K_{i,j}: read counts for gene i in the cfRNA of patient j;
μ_{i,j}: mean read counts for gene i in the cfRNA of patient j;
αᵢ: dispersion for gene i;
γᵢ: the mean reads count when no tumor contents in plasma for gene i;
x_{i,j}: tumor contents, log 10 (tumor fraction in matched cfDNA * gene expression in matched tumor tissue)
βᵢ: the coefficient for tumor contents;

K_{i,j}˜NB(μ_{i,j},αᵢ)

log(μ_{i,j})=(γᵢ+x_{i,j}βᵢ)

Feature selection using an information gain method was also tested. Information gain is a method to select genes with high mutual information between the binarized cfRNA gene expression and the cancer/non-cancer label. The gene expression RPM matrix was converted to a binary matrix. If the gene had an RPM>0, it was converted to 1. If the gene had an RPM=0, it was set to 0. The information gain was computed for each gene given the cancer type (e.g., lung cancer) and non-cancer label using the binary expression value. The non-cancer group for the breast cancer group was balanced with gender-only the female subjects in the non-cancer group were selected. The top 100 genes with the highest information gain were selected as the feature for modeling. The value of each gene was converted to binary value in the modeling process. These procedures were repeated for breast cancer vs. non-cancer, and colorectal cancer vs. non-cancer. The top 30 genes with the highest information gain for lung cancer are shown in Table 5, and the top 30 genes with the highest information gain for breast cancer are shown in Table 6.

In another embodiment, feature selection was carried out from cancer tissue samples to identify genes expressed in cancer tissues samples but not expressed in non-cancer participants. Libraries were prepared and sequenced as described above in Example 1. For each cancer tissue sample, we identified genes that were expressed at relatively high levels in cancer tissue (tissue RPM>10) from Dark Channels. These genes were classified as “tissue bright channel genes.” The top 15 tissue bright channel genes identified are shown in Table 7.

### Example 3: Validation of DCB's in a Separate Cohort

We set out to validate the DCBs identified in our CCGA cohort in an orthogonal set of breast (38) and lung (18) cancer samples obtained from a commercial vendor (Discovery Life Sciences, “DLS”). Stage I-IV patients were selected to assess the prevalence of DCBs across disease progression, and 38 age-matched non-cancer samples were included as controls of DCB expression in patients without cancer. In order to improve sensitivity and reduce sequencing requirements, we developed a targeted enrichment approach to select for 23 DCBs identified in our CCGA cohort. We also enriched for 33 positive control genes that are normally present in non-cancer plasma. These transcripts act as carrier material in the enrichment step, since the majority of non-cancer samples will not contain DCB transcripts. The resulting targeted RNA-seq libraries were sequenced and subsampled to a depth of 100M paired-end reads per sample, and the number of strict RNA reads quantified for both target and off-target genes. When compared to the whole transcriptome assay, we found that the targeted approach increased conversion efficiency for targeted cfRNA transcripts by 2- to 3-fold.

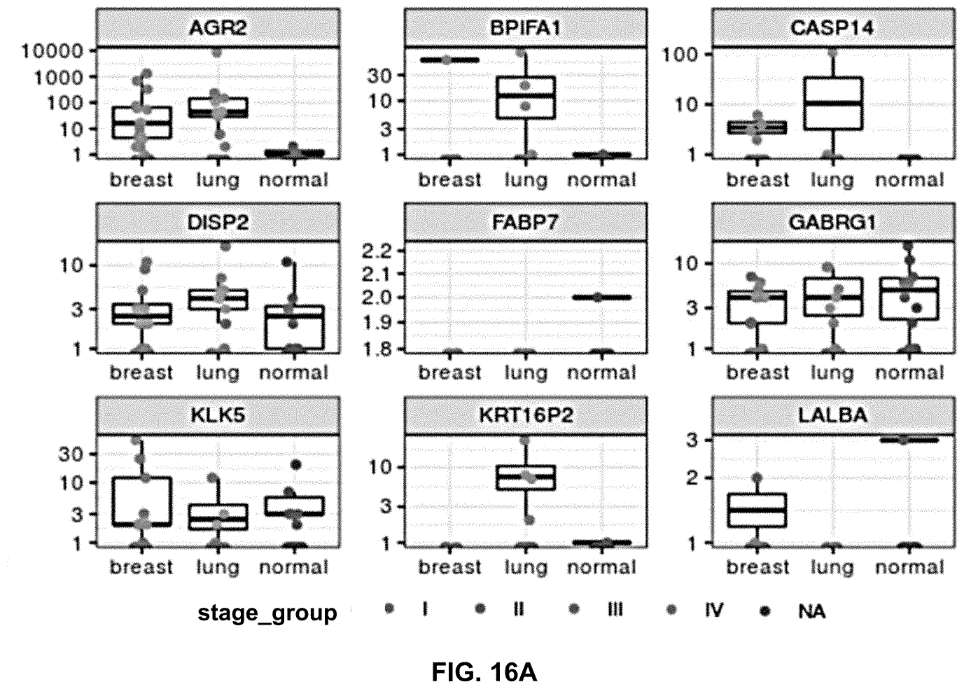

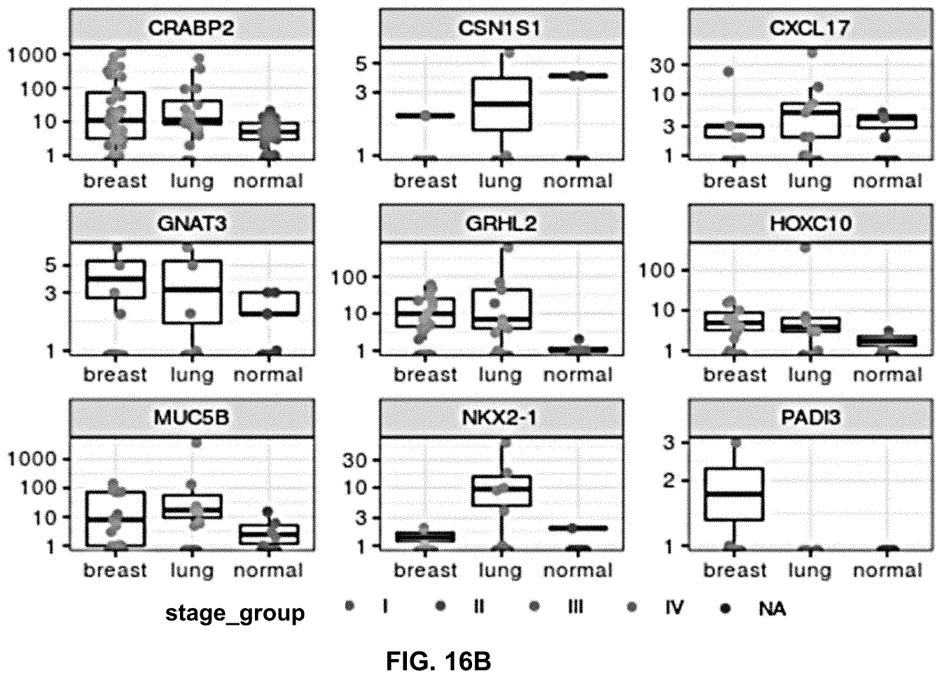

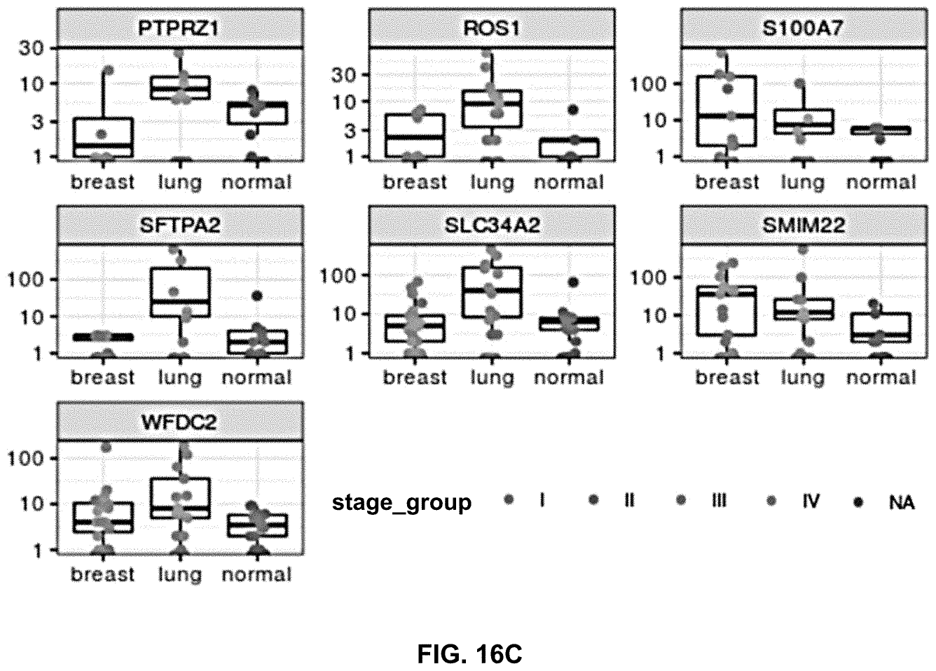

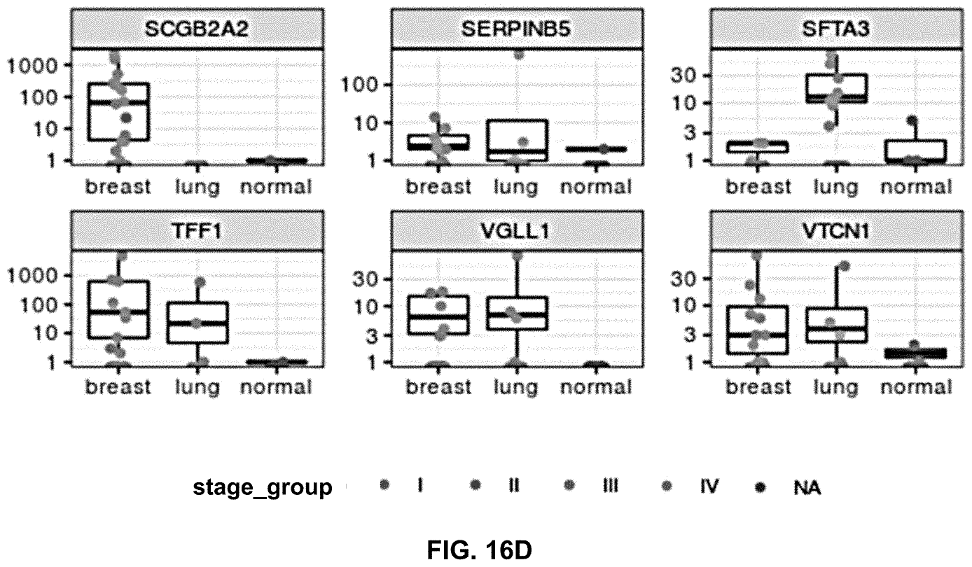

Of the 23 DCBs identified in our CCGA cohort, all but one (CRABP2) had a median expression (in RPM) of 0 in the non-cancer group. 19 DCBs in our panel were expressed in at least 1 cancer sample in the validation cohort (>2 unique fragments), and 16 of these DCBs were differentially expressed in at least one cancer type compared to non-cancer samples. With the increased assay efficiency and stage, we noticed that some tissue-specific markers are present in both breast and lung cancer, though they remain differentially expressed between the two groups. There are also some DCBs that are exclusively expressed in one cancer type, like SCGB2A2 in breast cancer, and ROS1, SFTA3, and SFTPA2 in lung cancer. For all of the DCBs observed in this validation cohort, the level of DCB expression in cancer samples increased with stage, with the highest expression seen for stage IV samples in our cohort, supporting the validity of these features as specific markers of cancer. Despite this trend, we also observed DCB expression in early stage cancers within our cohort, suggesting an opportunity to detect early stage cancers using an approach that enriches for DCBs. Illustrative results are shown in FIGS. 16A-D, with the number of read counts along the y-axis.

### Example 4: Classification Results

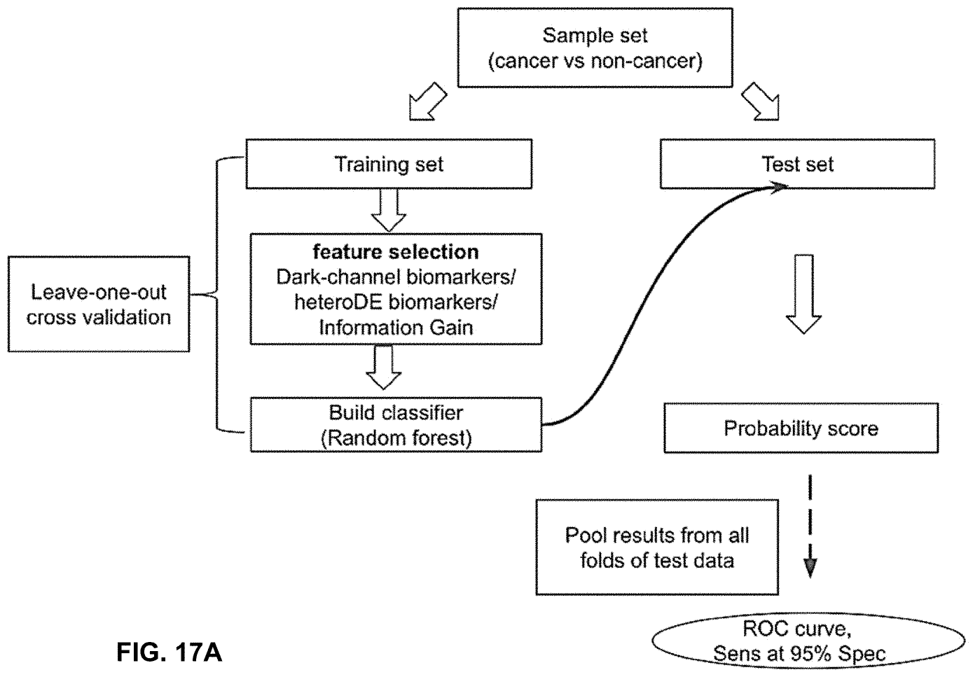

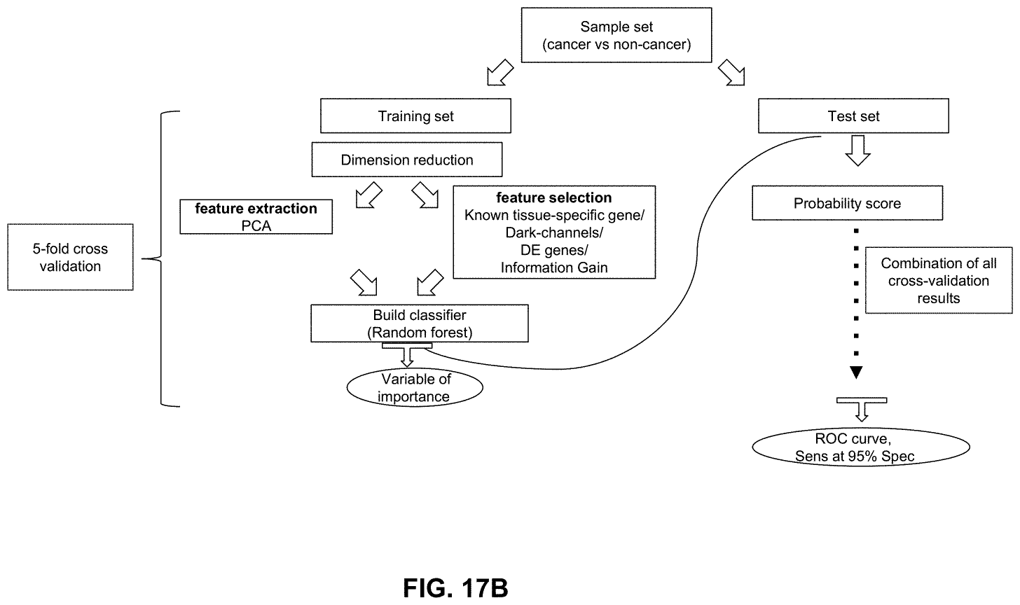

We applied leave-one-out (LOO) and 5-fold cross validation classification using different feature selection methods, including dark-channel biomarkers (DCB), heteroDE, and information gain (IG). Illustrative workflows are shown in FIGS. 17A-B. Because heteroDE utilized matched tumor tissue, this feature selection method was not applied to lung cancer/non-cancer classification due to limited number of lung tissue samples. Overall, LOO had significantly better classification performance in LOO compared to 5-fold cross validation in breast cancer/non-cancer classification, implying that the breast cancer classifier is under trained in 5-fold classification due to smaller sample sizes in each training set. DCB had the best performance (sensitivity at 98% specificity: 0.2±0.037) for lung cancer/non-cancer classifier and heteroDE had the best performance (sensitivity at 98% specificity: 0.303±0.046) for breast cancer/non-cancer classifier (Table 10).

TABLE 10

| Cancer Type | Feature Selection | Cross-Validation | Sens95spec |
|---|---|---|---|
| Lung | DCB | LOO | 0.3 ± 0.042 |
| Lung | IG | LOO | 0.333 ± 0.043 |
| Breast | heteroDE | LOO | 0.394 ± 0.049 |
| Breast | DCB | LOO | 0.212 ± 0.041 |
| Breast | IG | LOO | 0.303 ± 0.046 |
| Lung | DCB | 5-fold | 0.261 ± 0.146 |
| Breast | heteroDE | 5-fold | 0.177 ± 0.142 |

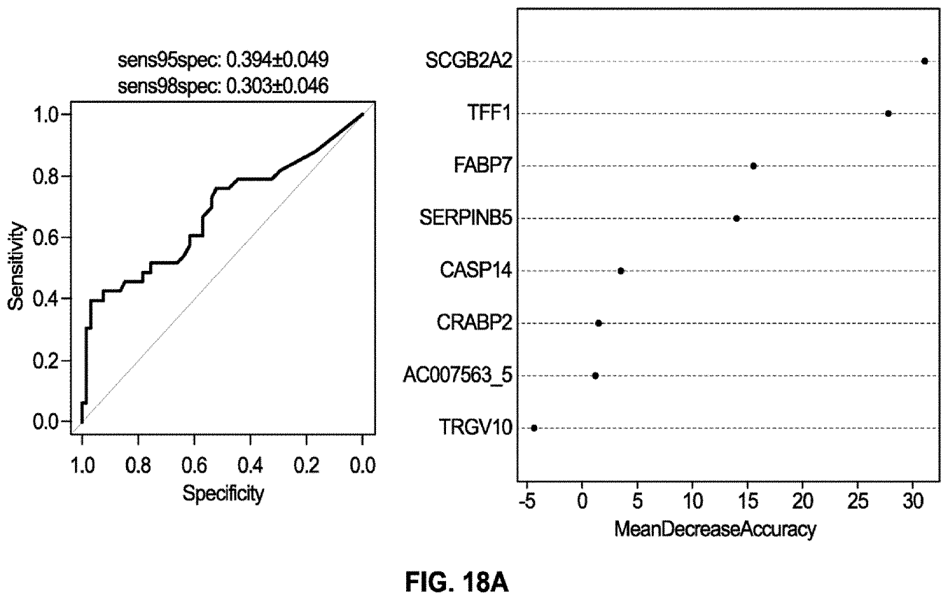

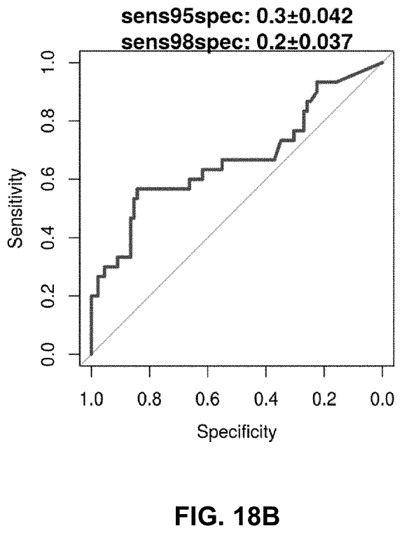

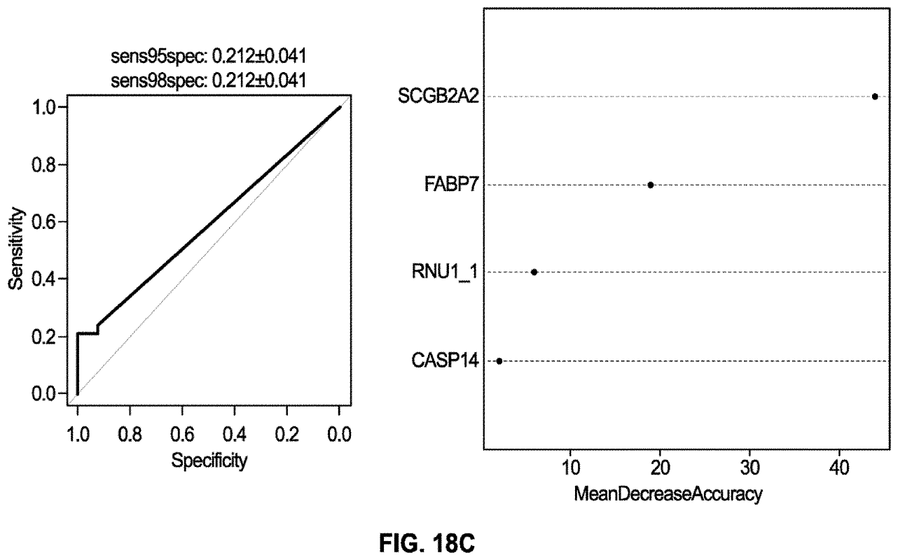

Illustrative results are also plotted in FIGS. 18A-C, which were generated using leave-one-out cross validation. FIG. 18A shows a receiver operating characteristic (ROC) plot and a variable importance plot from leave-one-out (LOO) cross-validation classification for breast vs non-cancer using the heteroDE feature selection method and a random forest classifier. The input data was counts per gene which was normalized using size factor normalization (using the estimateSizeFactors) function from the DESeq2 R package). As shown in Table 10, the sensitivity at 95% was 0.394+/−0.049.

FIG. 18B shows a ROC plot from leave-one-out (LOO) cross-validation classification for lung vs non-cancer labels using the dark channel feature selection method and a random forest classifier. The input data was normalized counts per gene in reads per million (rpm). As shown in Table 10, the sensitivity at 95% specificity was 0.3+/−0.042.

FIG. 18C shows a ROC plot and variable importance plot from leave-one-out (LOO) cross-validation classification for breast vs non-cancer labels using the dark channel feature selection method and a random forest classifier. The input data was normalized counts per gene in reads per million (rpm). As shown in Table 10, the sensitivity at 95% specificity was 0.212+/−0.041.

### Example 5: Materials and Methods

Sequencing Data Processing

Raw reads were aligned to gencode v19 primary assembly with all transcripts using STAR version 2.5.3a. Duplicate sequence reads were detected and removed based on genomic alignment position and non-random UMI sequences. A majority of paired-end reads had UMI sequences exactly matching expected sequences. A subset of reads contained errors in the UMI sequence and a heuristic error correction was applied. If the UMI was within a hamming distance of 1 from an expected UMI, it was assigned to that UMI sequence. In the case where hamming distance exceeded 1, or multiple known sequences were within a hamming distance of 1, the read with the UMI error was discarded. Sets of reads sharing alignment position and corrected UMIs were error corrected via multiple sequence alignment of member reads and a single consensus sequence/alignment was generated. Read alignments were compared to annotated transcripts in gencode v19. Only reads spanning annotated exon-exon junctions were counted to the remove false counts resulting from DNA contaminating reads.

Sample Collection

Whole blood was collected in Streck Cell-free DNA BCT tubes, which were shipped and stored at ambient temperature prior to plasma separation. Whole blood was spun at 1600 g for 10 min at 4° C. in a swing-bucket rotor to separate plasma. The plasma layer was transferred to a separate tube and spun at 15000 g for 12 min at 4° C. to further remove cellular contaminants. Double-spun plasma was stored at −80° C. and thawed at room temperature prior to extraction to avoid the formation of cryoprecipitates.

Sample Selection Criteria

We selected a subset of stage III breast, lung, and colorectal cancer samples from the Circulating Cell-free Genome Atlas study (CCGA, NCT02889978). We required that the selected patients had at least two tubes of unprocessed grade 1-2 plasma (no hemolysis), with 6-8 mL of plasma per patient. We further required that selected patients had matched cfDNA sequencing data from previous studies. Once the cancer patients were selected, we selected an equal number of non-cancer samples matched for age, gender, and ethnicity to the cancer samples. Based on this criteria, we selected 210 samples. These samples were randomized into batches of 14 using a randomization function in R that ensured a random mixture of cancer types (cancer and non-cancer samples) within each batch.

Sample Processing

Cell-free nucleic acids were extracted from up to 8 mL of frozen plasma using the circulating miRNA protocol from the QIAamp Circulating Nucleic Acids kit (Qiagen, 55114). The extracted material was DNase treated using the RNase-free DNase Set (Qiagen, 79254) according to the manufacturer's instructions and quantified using the High Sensitivity RNA Fragment Analyzer kit (Agilent, DNF-472). Reverse transcription and adapter ligation was performed using the TruSeq RNA Exome kit (Illumina, 20020189). The resulting libraries were depleted of abundant sequences using the AnyDeplete for Human rRNA and Mitochondrial Kit (Tecan, 9132), supplemented with a custom set of depletion targets.

Sequenced samples were screened and those exhibiting low quality control metrics were excluded from subsequent analysis. One assay metric and three pipeline metrics were chosen as “red flags” and were used to exclude samples with poor metrics. The assay metric measured whether samples had sufficient material for sequencing, and the pipeline metrics were sequencing depth, RNA purity, and cross-sample contamination.

Gene Expression Quantification

Initial inspection of the data revealed varying levels of residual DNA in cfRNA samples despite the DNase digestion step during library preparation. The level of contamination was minimal (<6 haploid genome equivalents per sample), and was not correlated with the amount of cfDNA prior to digestion or batch-specific issues. Rather, it appears to be stochastic, in line with previous reports.

A QC metric, “quantile 95 strand specificity” defined as the strand specificity of genes at or below the 95th quantile of expression, was used to assess the level of DNA contamination in each sample. UHR positive control samples exhibited high quantile 95 strand specificity (>0.85). cfRNA quantile 95 strand specificity values were spread across a wide range (0.52-0.89). For reference, cfDNA samples have a quantile 95 strand specificity of ˜0.5, suggesting that some cfRNA samples are dominated by signal from residual DNA. The read strand colors show even distribution of sense and anti-sense reads in NC67 versus only sense reads in NC3. Additionally, there is abundant coverage across both introns and exons in NC67, as would be expected with presence of DNA. The distribution of fragment length in samples with high levels of DNA contamination shows that they mimic the length distribution of cfDNA (median ˜160), strongly suggesting that undigested cfDNA is the major contaminant.

Samples with quantile 95 strand specificity below 0.84 were flagged and removed from subsequent analysis. To further guard against the inflation of RNA counts due to DNA contamination, the gene counts presented here are generated using strict counts, defined as read pairs where at least one of the two reads maps across an exon-exon junction. An experiment performed using varying levels of cfDNA spiked into a cfRNA sample showed that the estimation of RNA levels using strict counts remains unchanged, supporting the use of strict counts in the pilot study samples for quantifying and comparing gene expression.

Dark-Channel Features Election

The dark channel genes were identified by the following criteria: 1) The median expression (in RPM) of this gene in the non-cancer group is 0, and the standard deviation of this gene is less than 0.1 RPM. The dark channel biomarkers (DCB) for each cancer type were identified using the following criteria: 1) There are at least two samples in the specified cancer group for which the gene is expressed, 2) the RPM of the second highest expressed sample is greater than 0.1, and 3) the gene is differentially expressed in the specified cancer group compared to the non-cancer group (p-value <2e-02 for lung cancer and p-value <2e-01 for breast cancer). The p-value of two-group differential expression was calculated by the edgeR package. There are 816 genes with FDR<0.05 between lung cancer and non-cancer groups. There are 28 genes with FDR<0.05 between breast cancer and non-cancer groups. There are 4 genes with FDR<0.05 between colorectal cancer and non-cancer groups. For the boxplot and heatmap, we only displayed the most significant differentially expressed genes (FDR<2e-06 for lung and breast cancer and FDR<2e-02 for colorectal cancer).

Annotation of tissue-specific genes was performed as follows. The tissue-specific gene files for lung, breast, and colon cancers were downloaded from the Human Protein Atlas website (www.proteinatlas.org/). Tissue-specific genes are divided into three categories: 1) Tissue Enriched: At least 4-fold higher mRNA levels in a particular tissue as compared to all other tissues, 2) Group Enriched: At least 4-fold higher mRNA levels in a group of 2-5 tissues, 3) Tissue Enhanced: At least 4-fold higher mRNA levels in a particular tissue as compared to average levels in all tissues. All three categories were included in our definition of tissue-specific genes.

In order to test enrichment of the tissue-specific genes. 1) Fisher's exact test was applied to test the independence between lung DCB and lung-specific genes for all the annotated human genes. 2) Fisher's exact test was applied to test the independence between breast DCB and breast-specific genes for all the annotated human genes.

### Example 6: Panel of cfRNA Cancer Biomarkers

A study was designed to identify lung- and breast-cancer specific cfRNA biomarkers from the whole transcriptome distinct from a normal non-cancer cohort, and to identify biological signals represented specifically in cfRNA from cancer samples that may be useful for cancer binary detection and identifying tissue-of-origin (TOO) from plasma. We focused our work to identify gene features relevant to cancer subtypes that can be difficult to detect at early stages, namely lung adenocarcinoma and HR+ and triple negative (TNBC) breast cancers.

Data used to perform this analysis included 1) whole transcriptome plasma data sequenced from CCGA and from a commercial vendor, 2) whole transcriptome tissue data from TCGA, and 3) gene annotations from the Human Protein Atlas (Uhlén et al, Science 2015). A subset of stage III breast and lung cancer samples were selected and sequenced from the Circulating Cell-free Genome Atlas study (CCGA, NCT02889978). Stage III samples were selected to maximize signal in the blood while avoiding confounding signal from potential secondary metastases. In total, we analyzed 47 breast cancer, 14 lung adenocarcinoma cancers, and 93 non-cancer plasma samples from CCGA. Additionally, we included an additional set of whole transcriptome samples sourced from a commercial vendor (Conversant). This included a set of 14 stage IV breast cancer plasma samples, included to capture late stage signals of biomarkers in the blood. These plasma-derived data were used to define what genes are expressed in healthy plasma, and which are differentially expressed in cancer plasma that might be valuable for binary detection of cancer in these subtypes. We compiled the gene expression for each sample into an RPM (reads per million) normalized gene feature matrix, where each sample is a column and each row is a gene feature.

Also included in this study are breast cancer (BRCA) and lung adenocarcinoma (LUAD) tissue whole transcriptome data from the TCGA consortium, downloaded from the GDC portal. In total, this included 533 lung adenocarcinomas and 1102 breast cancer samples across stages I-IV. These data were used to identify high-expressing tumor-derived gene features for binary detection. Additionally, this high dimensional data was useful for identifying tissue-specific gene features that could be used for TOO. We compiled the gene expression for each sample into an RPM (reads per million) normalized gene feature matrix, where each sample is a column and each row is a gene feature.

Finally, we queried all the gene features in the Human Protein Atlas, which is an open-access compilation of various omics technology (transcriptomic and antibody-based) from cancer tumor samples and healthy tissue and provides tissue compartment and disease annotations. We used these annotations to capture whether the gene is cancer type enriched/enhanced, and favorable/unfavorable for disease prognostics, based on expression levels in tumor at diagnosis and overall survival rates of patients.

In order to establish a set of targets for binary detection and TOO classification, we first assessed if we could likely use TCGA tissue expression data downloaded from the GDC data portal to select likely biomarkers. For each gene, we calculated the mean gene expression across samples in both cohorts, and computed the Pearson correlation across the cohorts. Generally, we found that high mean gene expression in TCGA tissue roughly correlates with high mean gene expression in CCGA plasma (Spearman's rho of 0.568 for breast cancers, and 0.509 for lung cancers). Thus, we reasoned that TCGA tissue data can be informative for feature selection. We prioritized gene features with mean TCGA tissue expression greater than 1 RPM as likely detectable in cancer-derived plasma, and potentially informative for either binary cancer detection or tissue-of-origin detection. After filtering these for likely common artefact-inducing transcripts (transcripts mapping to HLA, IGH, IGL, and ribosomal genes), this resulted in 2898 potential gene features.

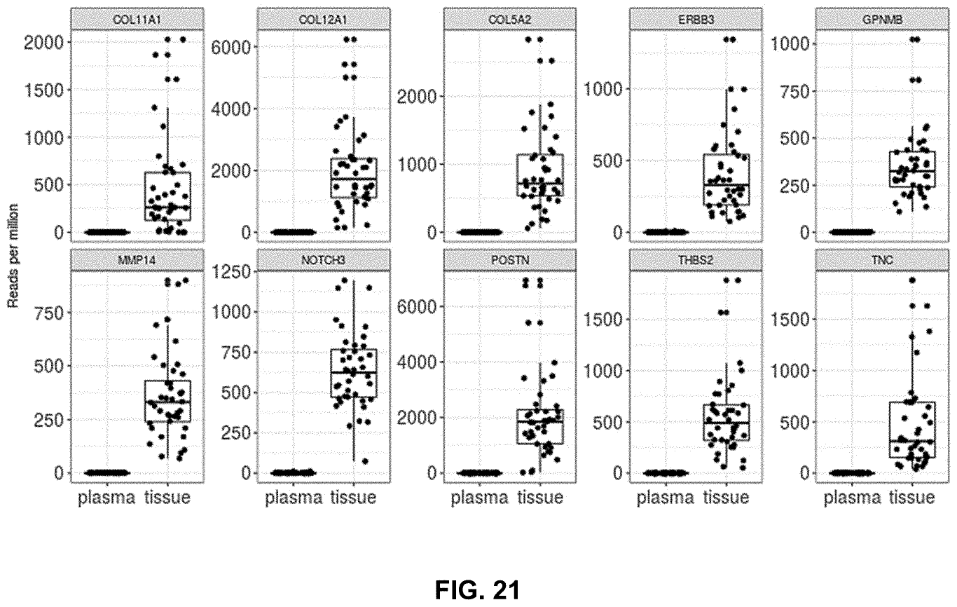

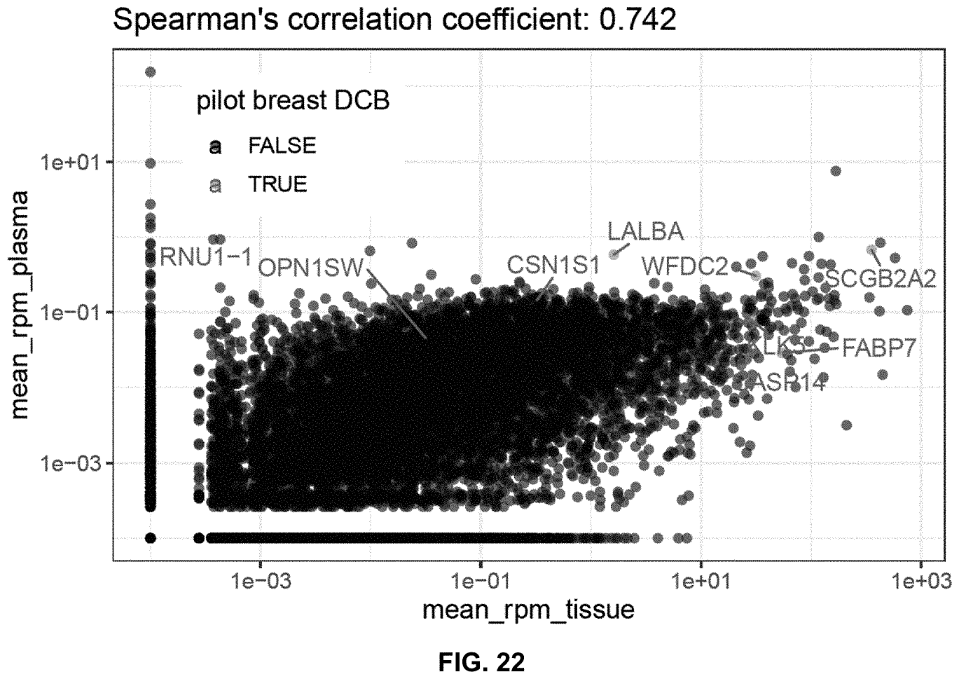

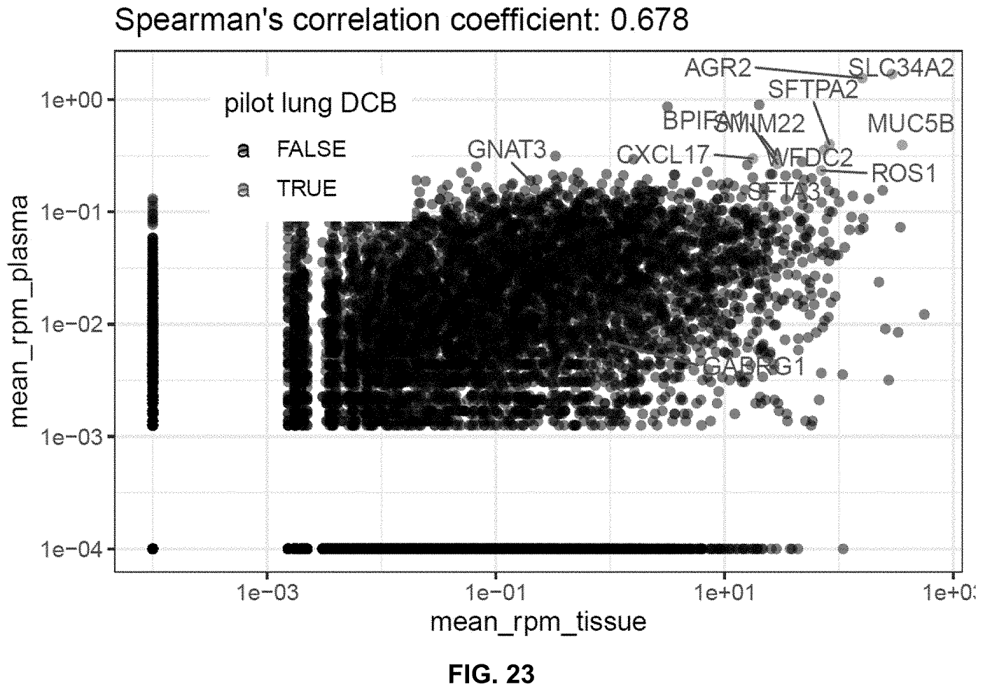

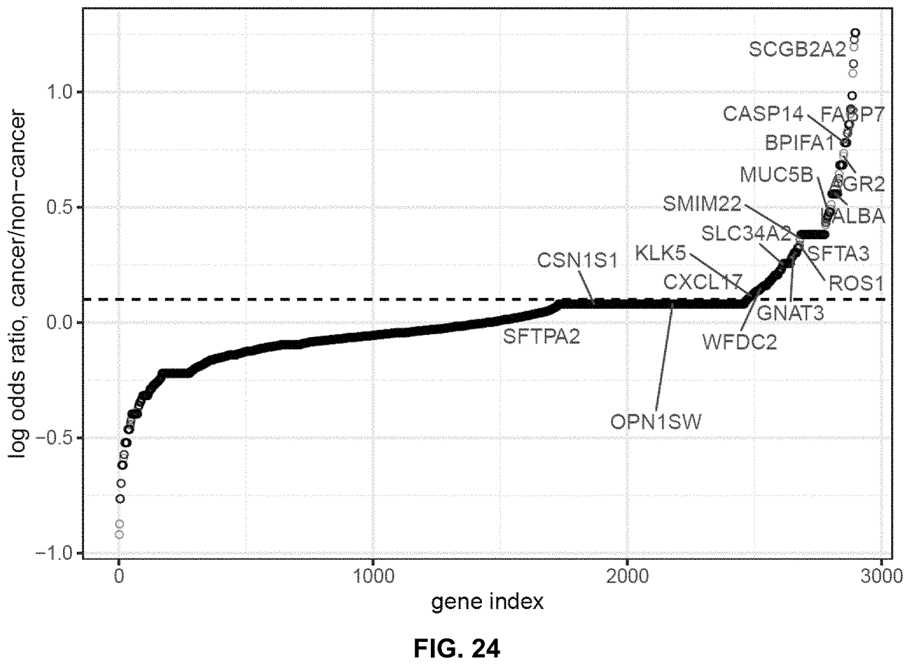

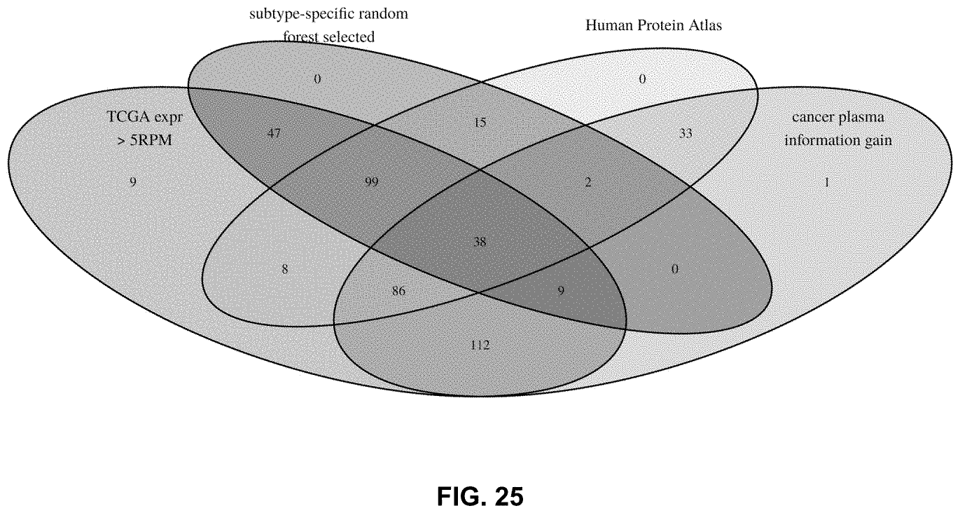

However, even though these gene features were highly expressed in the TCGA tissue, it was uncertain how prevalent these gene features were expressed in the plasma. Plots of mean RPM in tissue as compared to plasma are shown in FIG. 22 (breast cancer) and FIG. 23 (lung cancer). FIG. 21 provides example results for genes expressed at high levels in cancer tissue samples, with little to no detectable transcripts in plasma. Gene feature selection was also conducted leveraging information gained from expression in the plasma from CCGA. We binarized gene expression features as detected or not detected in the CCGA plasma samples, detected being expression at or above 0.005 reads per million (RPM). We then computed the plasma log odds ratio (LOR) for each gene based on observations from all cancer plasma to all non-cancer plasma. This quantifies the likelihood that a gene will occur in a cancer sample over the likelihood that the gene will occur in a non-cancer sample. An LOR>0 indicates greater likelihood of a gene being detected in cancer cases versus non-cancer cases, and LOR<0 indicates a likelihood of a gene being detected in non-cancer cases versus cancer cases. We selected the most informative genes in the plasma with an LOR>0.1, resulting in 281 gene features. An example plot of LOR for cfRNA biomarkers is shown in FIG. 24.

Further, we set out to assess which gene features are useful specifically for TOO classification. Since the CCGA dataset for cfRNA is limited to <200 samples, we determined to use the TCGA tumor gene matrix and perform a recursive feature elimination algorithm to identify gene features that are important for differentiating between lung adenocarcinoma, breast HR+, and breast TNBC cancers. A random forest multiclass model was used to recursively select top K genes with 10-fold cross validation across all gene features. Features are eliminated across iterations by optimizing accuracy across folds. The cross-validated model classifies the TCGA samples with 96.7% accuracy when using 750 gene features, so we identified these top 750 biomarkers as important for subtype classification in the tissue.

The Human Protein Atlas compiles TCGA transcriptomics and antibody-based protein data from cancer tumor samples as well as healthy tissue samples to provide two specific atlases we used to prioritize gene features for binary detection and TOO. The Tissue Atlas includes annotations for genes that are tissue enriched (elevated in tissue compared to other tissues) and tissue enhanced (expressed in tissue with low specificity), based on mRNA and protein levels in normal tissue. Additionally, the Pathology Atlas includes annotations for genes that are cancer type enriched (elevated in tumor type compared to other tumors) or enhanced (expressed in tumor type with low specificity), as well as favorable/unfavorable for disease prognosis, based on expression levels in tumor at diagnosis and overall survival rates of patients. We marked genes as potential biomarkers that had these annotations for breast and lung cancers (3028 genes features).

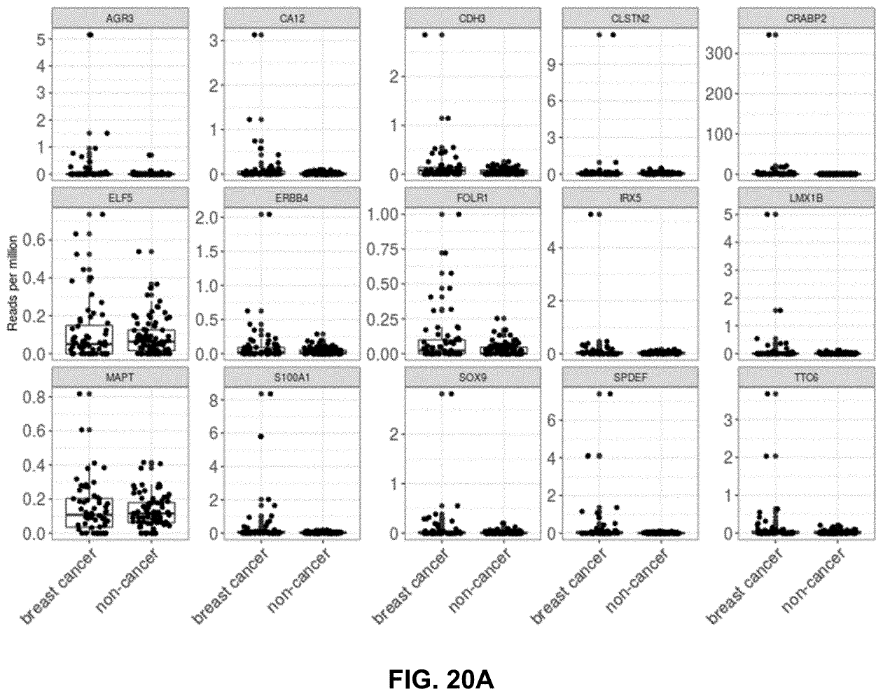

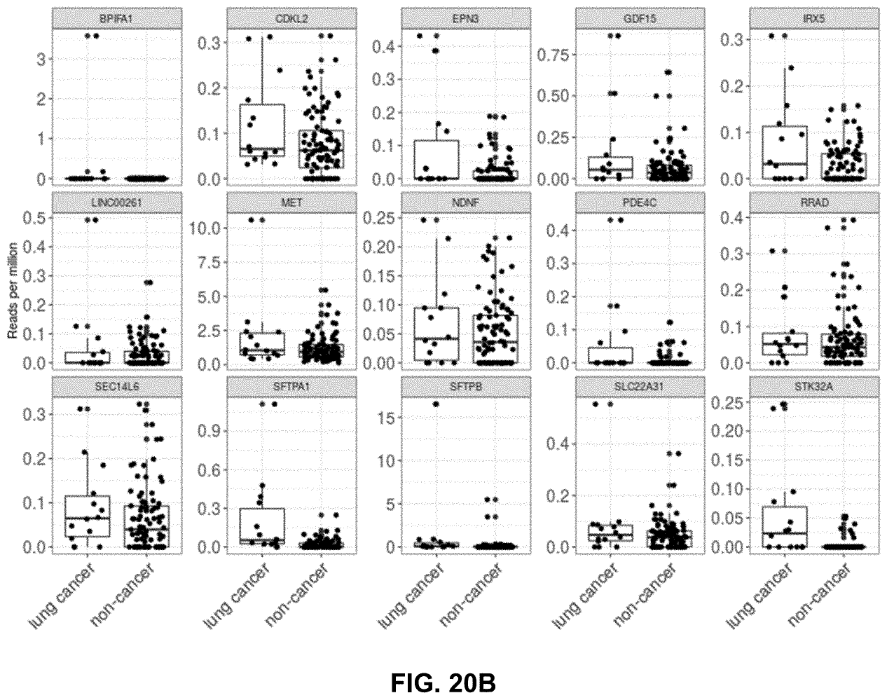

The majority of transcripts found in the plasma is thought to derive from healthy immune cells. To select biomarkers that are not present in healthy white blood cells, which can confound cancer detection, we filtered gene features to have low expression in plasma from healthy individuals from the CCGA cohort (median RPM<1, standard deviation RPM<0.1). These resulting 41391 gene features are referred to as “dark channels”. We further filtered these dark channels by integrating the aforementioned approaches at identifying binary cancer detection and TOO biomarkers. The dark channels were filtered so that either the gene binarized LOR>0.1 for cancer-associated gene features, or the gene was included in the 750 genes selected by the random forest model. These genes were further filtered so that they were either annotated by the Human Protein Atlas or the mean expression was greater than 5 RPM in a TCGA cohort. Additional positive control and DCB genes from Examples 1-4 were added to this updated biomarker set, bringing the total number of cfRNA biomarkers to 467, which are listed in Table 15 (a subset of which are provided in Table 11). The genes of Table 14 represent a subset of particularly informative cfRNA biomarkers. Example results for selected biomarkers for breast and lung cancer are illustrated in FIGS. 10A and 20B, respectively.

### Example 7: Detection of Polypeptide Biomarkers

Using cfDNA and cfRNA data from the CCGA study, a protein panel was designed to enrich for genes of interest in a protein-based assay, and the compared to baseline protein levels in non-cancer plasma. In particular, polypeptides corresponding to cfRNA markers identified in the CCGA study were analyzed, including protein products of selected genes listed in Table 16B. Protein-based detection assays that may be used for this analysis include mass-spectrometry assays such as multiple reaction monitoring (MRM) mass spectrometry (e.g., by Caprion), proximity extension assays (e.g., by Olink), or affinity labeling assays such as magnetic nanoparticle protein coronas followed by mass spectrometry (e.g., by SEER).

In this example, polypeptides were detected by proximity extension assay (PEA). For each biomarker, a matched pair of antibodies linked to unique oligonucleotides barcodes, called proximity probes, simultaneously bind to the respective protein target. If the protein target is present in the sample, the proximity probes come in close proximity and hybridize to each other, forming a nucleic acid duplex that allows at least one of the nucleic acid domains to be extended from its 3′ end. The addition of a DNA polymerase leads to an extension of the hybridizing oligo, bound to one of the probes, which creates a DNA amplicon that can subsequently be detected and quantified by quantitative real-time PCR.

For the PEA assay, whole blood samples were collected in Streck Cell-free DNA BCT® tubes, which were shipped and stored at ambient temperature prior to plasma separation. Samples were from three sets of subjects: (1) a first set of subjects from the CCGA study (“CCGA1,” n=38), (2) a second set of subject from the CCGA study (“CCGA2,” n=393), and (3) a set of samples from Discovery Life Sciences (“DLS,” n=42). Subjects included those with breast or lung cancer, or those without a cancer diagnosis (“non-cancer”). For some analyses, samples were further subgrouped as those with tumor fraction below 0.3% (“low TF”), those with tumor fraction above 0.3% (“high TF”), and/or the type of cancer with which the subject was diagnosed (e.g., breast cancer or lung cancer). Whole blood was centrifuged at 1600×g for 10 min at 4° C. to separate plasma. The plasma layer was transferred to a separate tube and centrifuged at 15,000×g for 12 min at 4° C. to further remove cellular contaminants. Double-spun plasma was stored at −80° C. until further use. Around 40-80 uL of double-spun plasma was diluted and used as input into a proximity extension assay (PEA).

The levels of the target peptides were measured as counts based on the number of sequenced barcode reads corresponding to each target peptide. The ratio of protein counts to extension control counts was divided by the media ratio of plate control protein counts to extension control counts to generate normalized counts for each target peptide. Median normalized counts were calculated by dividing the normalized counts for each peptide by the median sample normalized counts. This sample-major normalization accounted for collection conditions and biological confounders (e.g., variation in total protein concentration, impact of meals or exercise of an individual on the collected sample, etc).

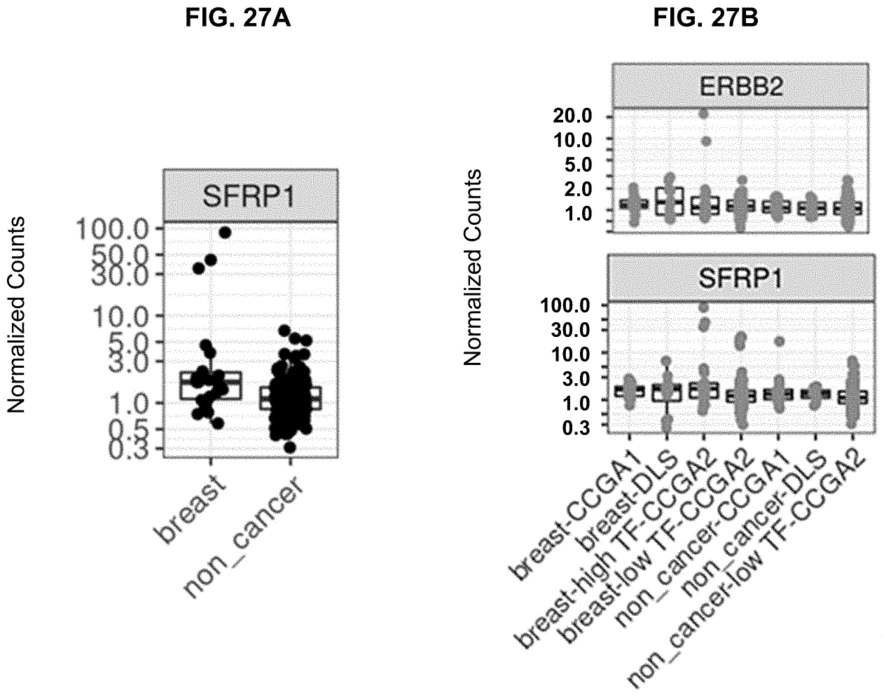

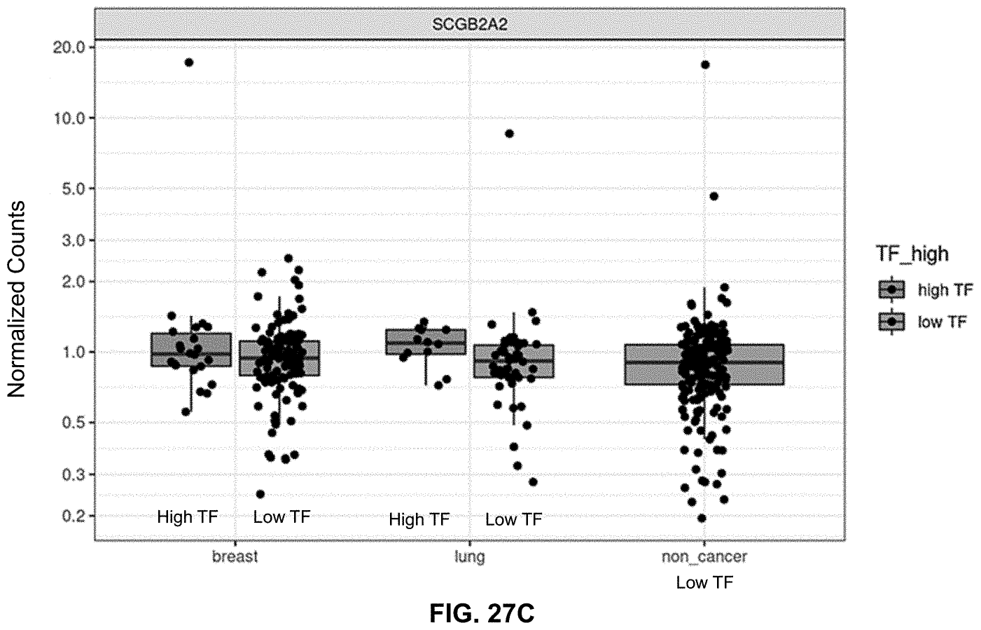

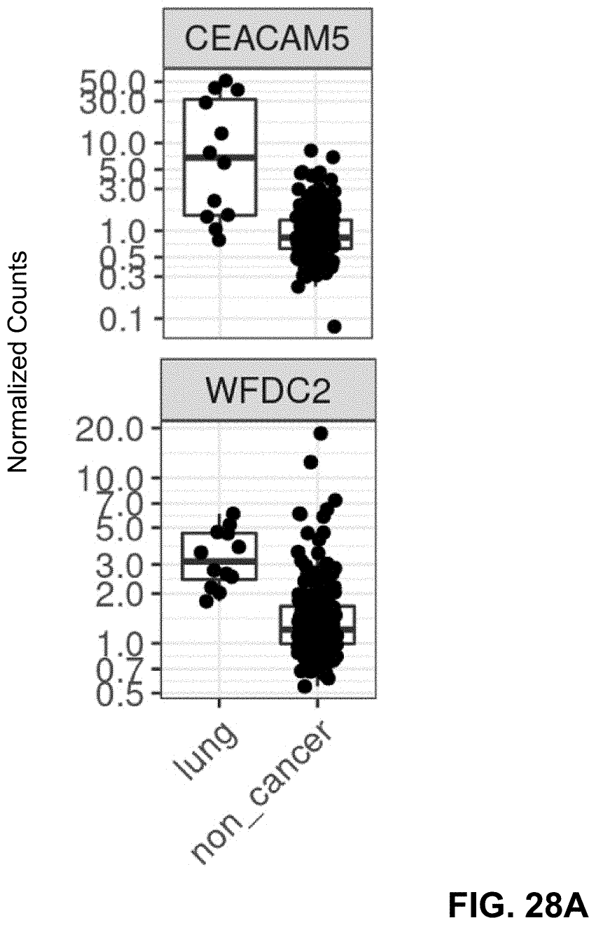

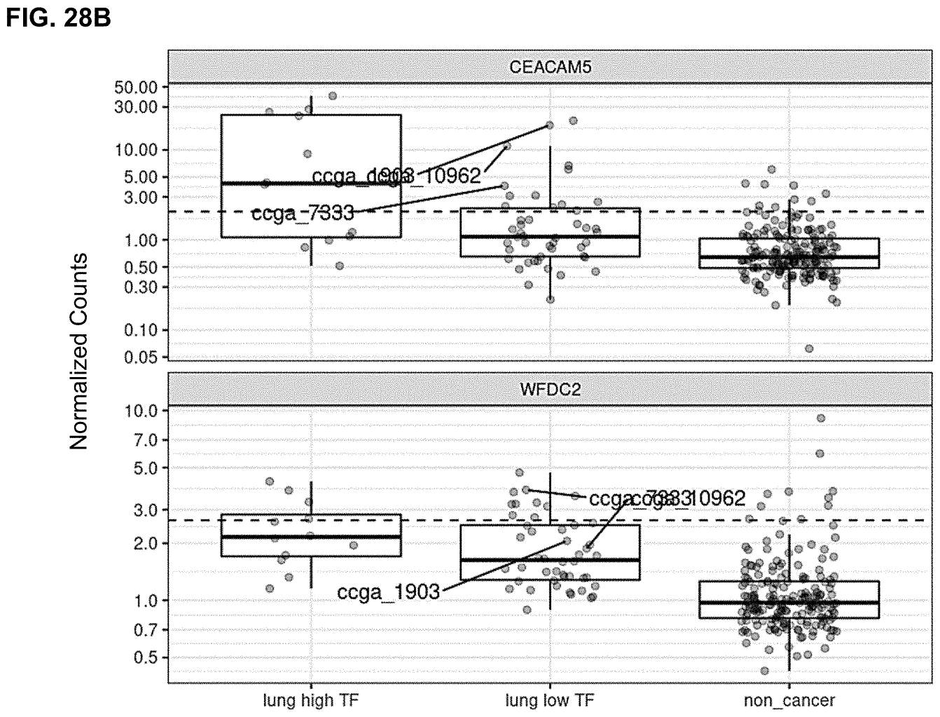

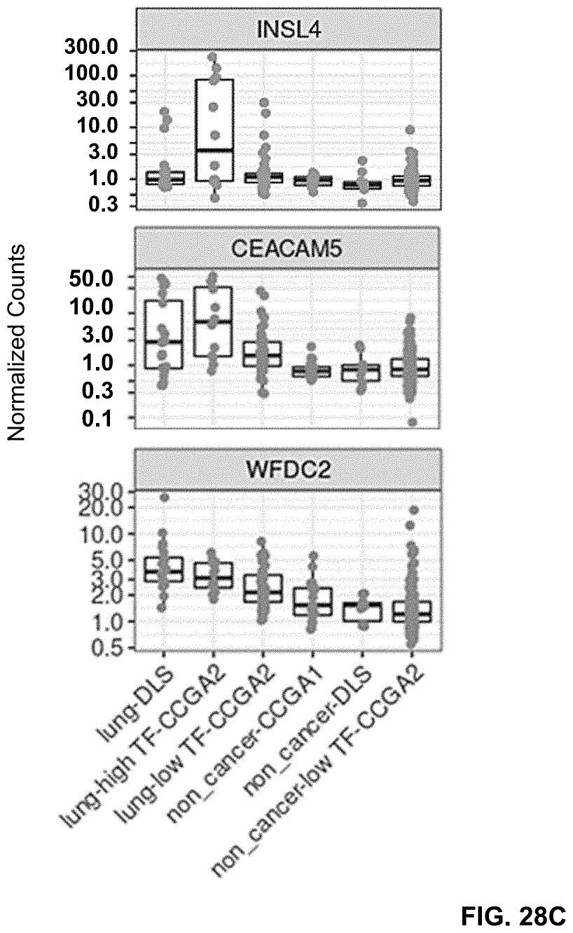

Samples were tested for proteins encoded by biomarker genes listed in Table 16B. The determined biomarker polypeptide expression levels were compared between cancer and non-cancer plasma samples to define a threshold for characterizing the cancer status, particularly for breast and lung cancer. Exemplary results from this analysis are shown in FIGS. 27A-27C for breast cancer, and FIGS. 28A-28C for lung cancer. Circulating WFDC2, CXCL17, MMP12, GDF15, CEACAM5, PRSS8, TFF1, CWC15, ALPP, GP2, INSL4, CHGA, GFRA1, AGR2, SPON1, DXO, AIF1, FKBPL, SFTPA2, and FOLR1 proteins were found to be useful in distinguishing between non-cancer and lung cancer status in plasma samples, and were statistically significant at the p<0.05 level (see, e.g., FIGS. 28A-28C). Circulating ADAMTS15, LEP, ERBB2, ERBB4, CGA, AFP, F7, BPIFB2, SFRP1, FGFBP1, LAMA4, GP2, MIA, FGFR2, and VTCN1 proteins were higher among breast cancer subjects as compared to non-cancer subjects, and found to be statistically significant at the p<0.05 level (see, e.g., FIG. 27A-27B). By comparison, the polypeptide level of mammaglobin, also known as SCGB2A2, was not found to distinguish on its own between the non-cancer and breast cancer subject samples in this example (FIG. 27C).

These results show that dark-channel cell-free RNA biomarkers for cancer identified herein are useful in selecting circulating polypeptide biomarkers for cancer, which are also likely to be more highly abundant in both tumor tissue and in circulation. Moreover, the levels of cfRNA biomarkers, as well as the polypeptides they encode, can be used to distinguish between cancer and non-cancer state in a subject, as well as to identify a cancer tissue of origin or cancer subtype.

### Example 8: Multi-Omics Detection of Cancer Using Polypeptides, cfDNA, and cfRNA

Low tumor fractions render the detection of early stage cancers in blood difficult. A multi-omics approach, which leverages different types of tumor-derived signal such as polypeptides, in conjunction with cfDNA and cfRNA, improves sensitivity and tissue of origin identification. Such an approach has the advantages of increasing cancer detection sensitivity for early stage cancers, resolving tissue of origin identification for cancers of unknown primary (CUP), and aiding in the identification of cancer sub-types using previously validated polypeptides markers.

Polypeptides represent the final step in the flow of genetic information. Target cfRNA molecules are amplified compared to cfDNA. Target polypeptides are also amplified, and are more long-lived than cfRNA. Polypeptides levels are correlated with cfRNA levels in the case of mammaglobin. Signal for cfRNA biomarkers described herein can be used to guide polypeptide biomarker selection, and detection of both cfRNA and polypeptides are used together to increase detection accuracy.

### Example 9: Evaluation of cfRNA Biomarkers in Cancer Samples

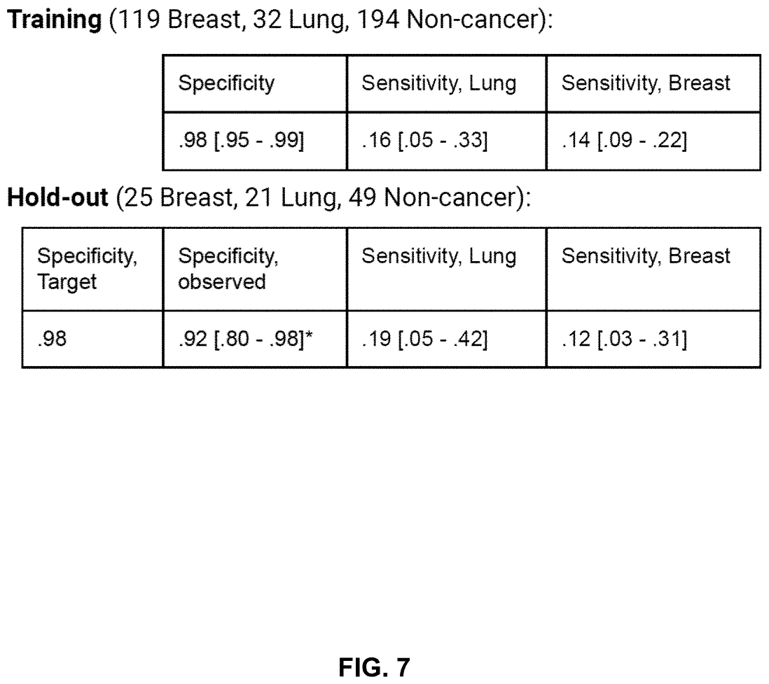

The 467 cfRNA biomarkers listed in Table 15 were tested for the ability to identify cancer in hard-to-detect breast and lung cancers with low tumor fraction, and distinguish non-cancers. All samples were scored based on the highest evidence observed in any gene in the sample. We selected all genes with some evidence of signal in high-signal cancers. For each sample, we identified all genes that have more evidence in that sample than in all other non-cancers, and ranked samples by the top-evidence gene in each sample, using the following criteria, in order: (1) max counts observed in any non-cancer (lower being better), (2) max counts observed in any high-signal cancer (higher being better), and (3) counts observed in that sample. A leave-one-out classifier was evaluated using these biomarkers in training and hold-out ample sets. Results are illustrated in FIG. 7. As indicated by the asterisk, the validation cohort specificity had a significant decrease (p=0.0.02), relative to the training cohort. Without wishing to be bound by theory, this may indicate potential overfit in this particular experiment.

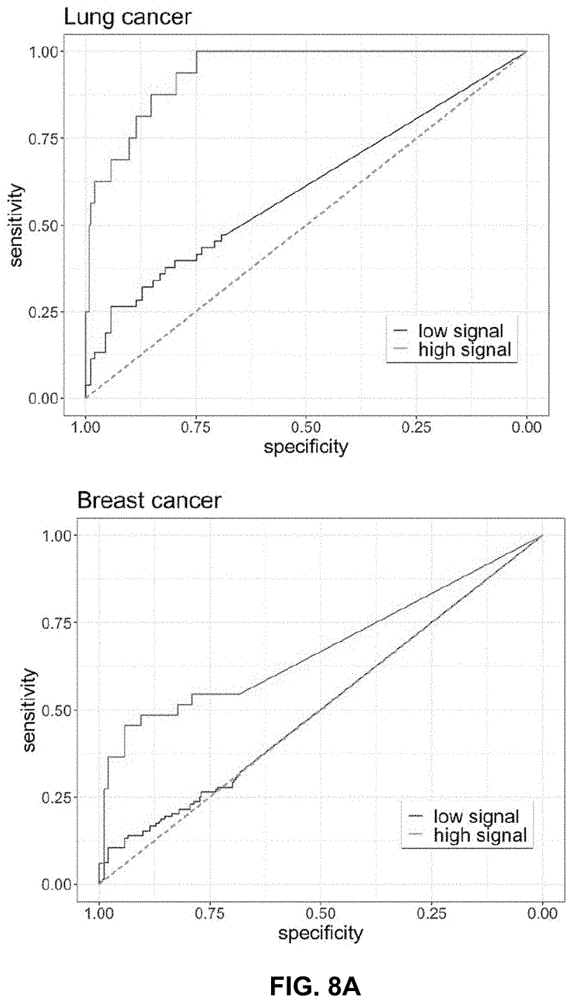

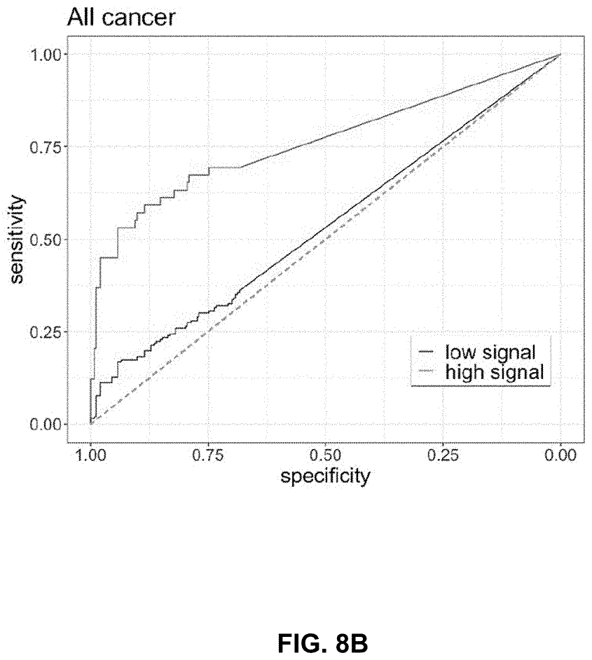

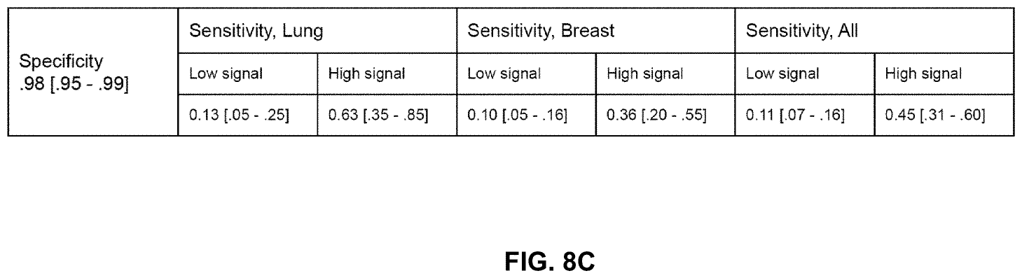

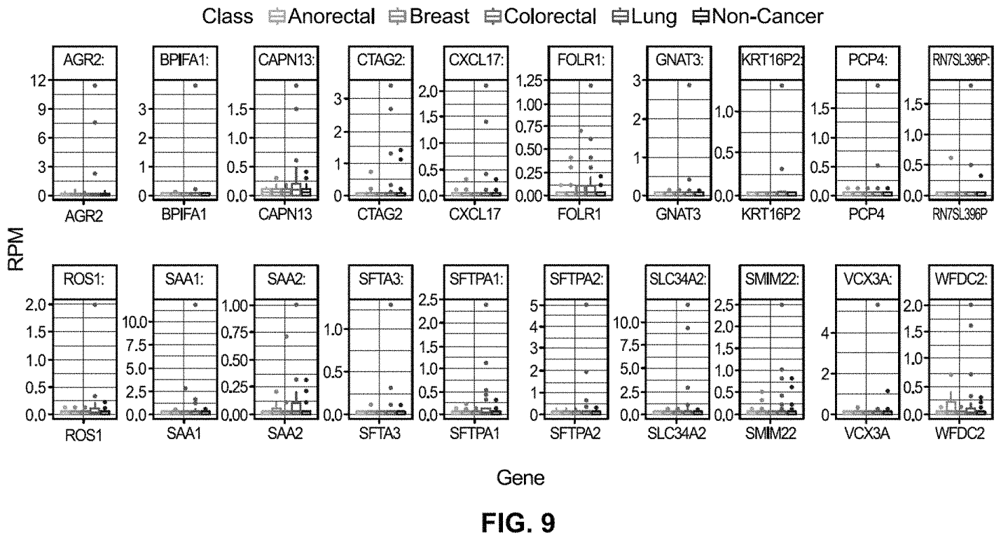

The leave-one-out classifier based on cfRNA biomarkers was applied to cancer samples having low or high signal for a DNA methylation cancer biomarker. Samples included lung cancer and breast cancer samples. The classifier demonstrated high specificity performance, as illustrated in FIGS. 8A-8C.

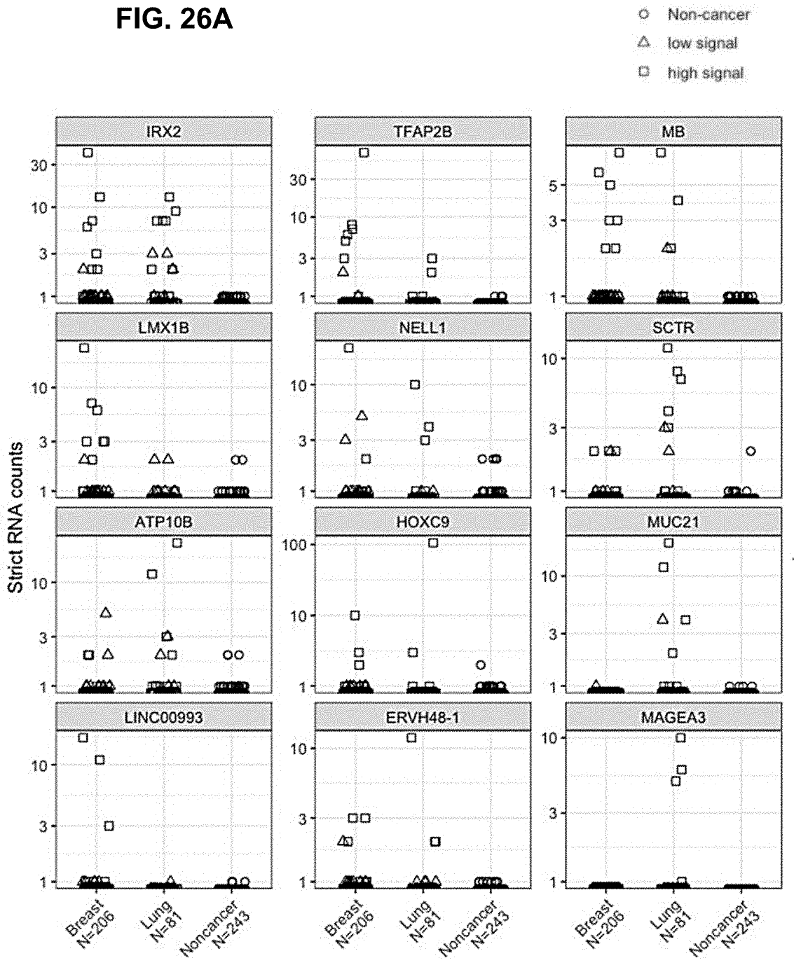

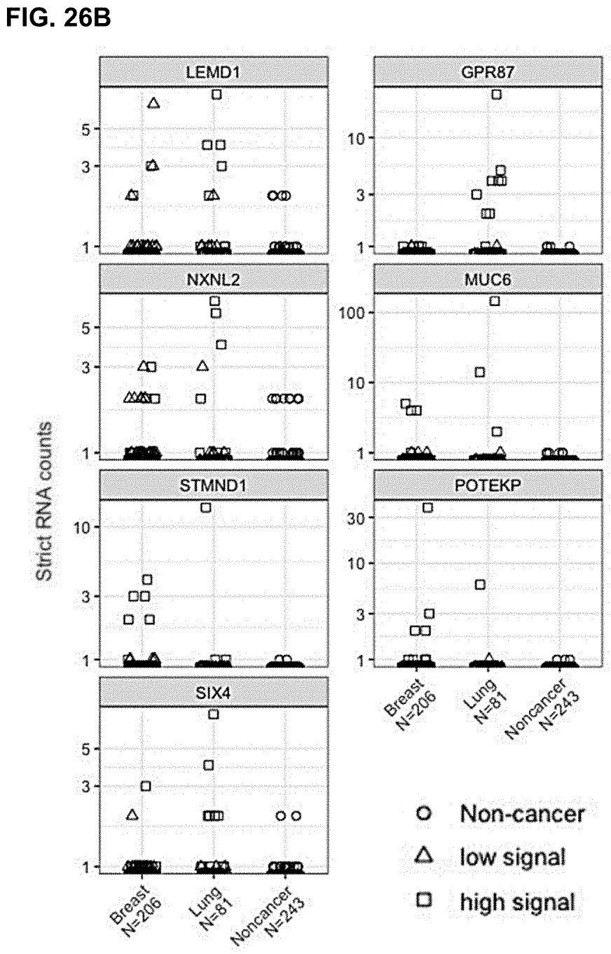

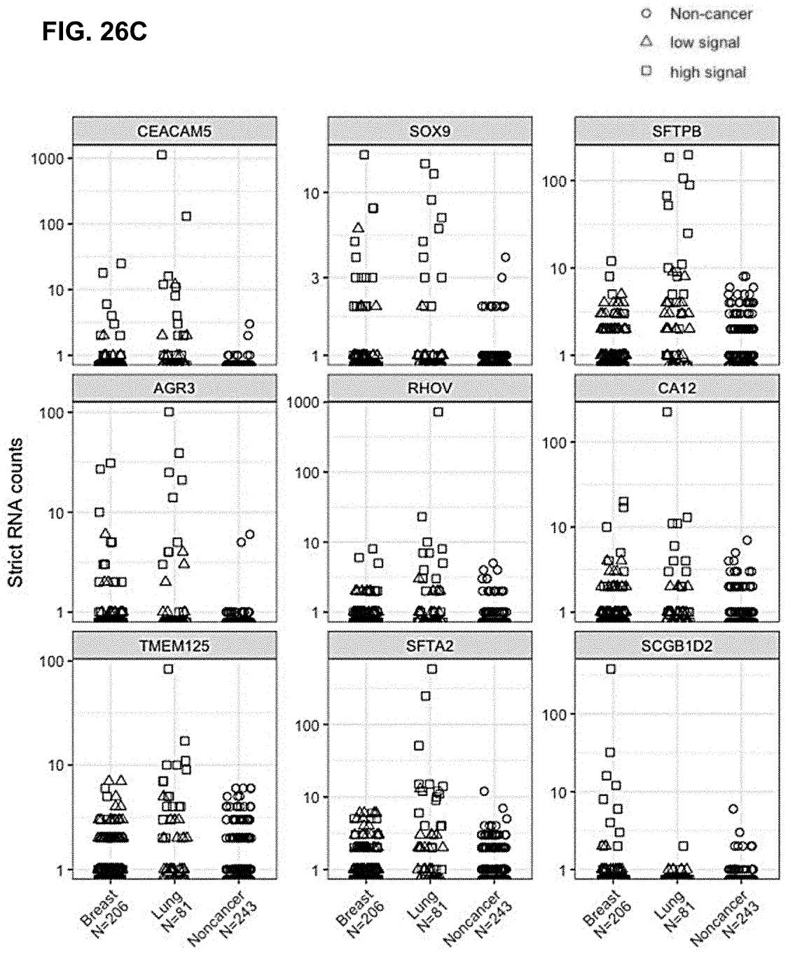

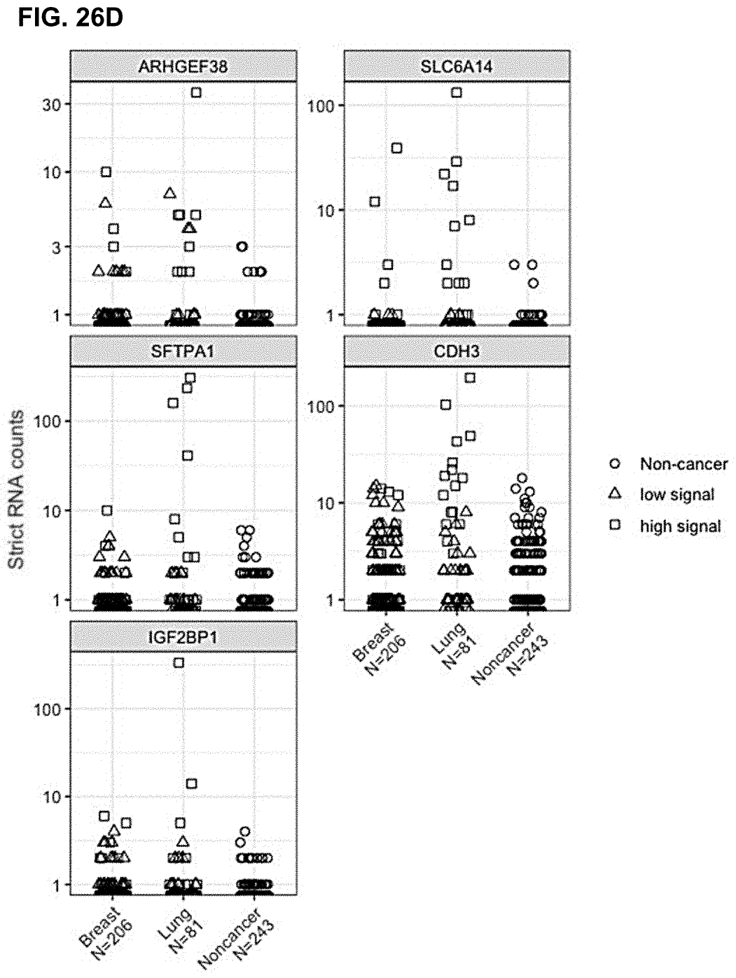

Several genes proved to be particularly informative cfRNA cancer biomarkers, some with specificity for breast cancer or lung cancer, and some being elevated in both breast and lung cancer. These 33 genes are listed in Table 8 above. The results are presented graphically for strict read counts in FIGS. 26A-26D. Additional details concerning results for these 33 genes are provided in Table 20 below.

TABLE 20

| | | | Number breast | Number lung |
|---|---|---|---|---|
| | Maximum | Maximum | cancers | cancers |
| Gene | high signal | non-cancer | detected* | detected* |
| Symbol | cancer count | count | (n = 206) | (n = 81) |
| CEACAM5 | 1125 | 3 | 4 | 8 |
| RHOV | 725 | 5 | 2 | 6 |
| SFTA2 | 589 | 12 | 0 | 7 |
| SCGB1D2 | 381 | 6 | 5 | 0 |
| IGF2BP1 | 335 | 4 | 2 | 3 |
| SFTPA1 | 305 | 6 | 1 | 5 |
| CA12 | 226 | 7 | 3 | 4 |
| SFTPB | 197 | 8 | 1 | 11 |
| CDH3 | 195 | 18 | 0 | 7 |
| MUC6 | 146 | 1 | 3 | 2 |
| SLC6A14 | 132 | 3 | 2 | 6 |
| HOXC9 | 106 | 2 | 2 | 2 |
| AGR3 | 101 | 6 | 3 | 5 |
| TMEM125 | 84 | 6 | 2 | 8 |
| TFAP2B | 65 | 1 | 6 | 1 |
| IRX2 | 41 | 1 | 5 | 7 |
| POTEKP | 38 | 1 | 2 | 1 |
| ARHGEF38 | 36 | 3 | 3 | 7 |
| GPR87 | 25 | 1 | 0 | 6 |
| LMX1B | 24 | 2 | 6 | 0 |
| ATP10B | 24 | 2 | 1 | 4 |
| NELL1 | 22 | 2 | 3 | 3 |
| MUC21 | 20 | 1 | 0 | 4 |
| SOX9 | 17 | 4 | 5 | 6 |
| LINC00993 | 17 | 1 | 3 | 0 |
| STMND1 | 14 | 1 | 3 | 1 |
| ERVH48-1 | 12 | 1 | 2 | 1 |
| SCTR | 12 | 2 | 0 | 6 |
| MAGEA3 | 10 | 0 | 0 | 3 |
| MB | 8 | 1 | 5 | 2 |
| LEMD1 | 8 | 2 | 3 | 4 |
| SIX4 | 8 | 2 | 1 | 2 |
| NXNL2 | 7 | 2 | 2 | 4 |

*Genes were called detected if strict RNA count was above the maximum non-cancer count or 2, whichever was higher.

### REFERENCES

Klein et al. Development of a comprehensive cell-free DNA (cfDNA) assay for early detection of multiple tumor types: The Circulating Cell-free Genome Atlas (CCGA) study. ASCO (2018).
Uhlén et al. Tissue-based map of the human proteome (www.proteinatlas.org). Science doi:10.1126/science.1260419 (2015).
A. M. Newman, et al., An ultrasensitive method for quantitating circulating tumor DNA with broad patient coverage. Nat. Med. 20, 548-554 (2014).
E. Kirkizlar, et al., Detection of Clonal and Subclonal Copy-Number Variants in Cell-Free DNA from Patients with Breast Cancer Using a Massively Multiplexed PCR Methodology. Transl. Oncol. 8, 407-416 (2015).
S. Y. Shen, et al., Sensitive tumour detection and classification using plasma cell-free DNA methylomes. Nature 563, 579-583 (2018).
C. Bettegowda, et al., Detection of circulating tumor DNA in early- and late-stage human malignancies. Sci. Transl. Med. 6, 224ra24 (2014).
K. C. A. Chan, et al., Noninvasive detection of cancer-associated genome-wide hypomethylation and copy number aberrations by plasma DNA bisulfite sequencing. Proc. Natl. Acad. Sci. U.S.A 110, 18761-18768 (2013).
I. S. Haque, O. Elemento, Challenges in Using ctDNA to Achieve Early Detection of Cancer. bioRxiv, 237578 (2017).
K. C. A. Chan, et al., Cancer genome scanning in plasma: detection of tumor-associated copy number aberrations, single-nucleotide variants, and tumoral heterogeneity by massively parallel sequencing. Clin. Chem. 59, 211-224 (2013).
C. Abbosh, et al., Phylogenetic ctDNA analysis depicts early-stage lung cancer evolution. Nature 545, 446-451 (2017).
K. W. Lo, et al., Analysis of Cell-free Epstein-Barr Virus-associated RNA in the Plasma of Patients with Nasopharyngeal Carcinoma. Clin. Chem. 45, 1292-1294 (1999).
M. S. Kopreski, F. A. Benko, L. W. Kwak, C. D. Gocke, Detection of tumor messenger RNA in the serum of patients with malignant melanoma. Clin. Cancer Res. Off J. Am. Assoc. Cancer Res. 5, 1961-1965 (1999).
J. D. Arroyo, et al., Argonaute2 complexes carry a population of circulating microRNAs independent of vesicles in human plasma. Proc. Natl. Acad. Sci. U.S.A 108, 5003-5008 (2011).
P. M. Godoy, et al., Large Differences in Small RNA Composition Between Human Biofluids. Cell Rep. 25, 1346-1358 (2018).
M. F. de Souza, et al., Circulating mRNAs and miRNAs as candidate markers for the diagnosis and prognosis of prostate cancer. PLoS ONE 12 (2017).
G. Y. F. Ho, et al., Differential expression of circulating microRNAs according to severity of colorectal neoplasia. Transl. Res. 166, 225-232 (2015).
I. Lee, D. Baxter, M. Y. Lee, K. Scherler, K. Wang, The importance of standardization on analyzing circulating RNA. Mol. Diagn. Ther. 21, 259-268 (2017).
X. Q. Chen, et al., Telomerase RNA as a detection marker in the serum of breast cancer patients. Clin. Cancer Res. Off J. Am. Assoc. Cancer Res. 6, 3823-3826 (2000).
17. R. C. Kamm, A. G. Smith, Ribonuclease activity in human plasma. Clin. Biochem. 5, 198-200 (1972).
T. El-Hefnawy, et al., Characterization of amplifiable, circulating RNA in plasma and its potential as a tool for cancer diagnostics. Clin. Chem. 50, 564-573 (2004).
N. B. Y. Tsui, E. K. O. Ng, Y. M. D. Lo, Stability of endogenous and added RNA in blood specimens, serum, and plasma. Clin. Chem. 48, 1647-1653 (2002).
J. D. Arroyo, et al., Argonaute2 complexes carry a population of circulating microRNAs independent of vesicles in human plasma. Proc. Natl. Acad. Sci. U.S.A 108, 5003-5008 (2011).
G. J. S. Talhouarne, J. G. Gall, 7SL RNA in vertebrate red blood cells. RNA 24, 908-914 (2018).
L. A. Hancock, et al., Muc5b overexpression causes mucociliary dysfunction and enhances lung fibrosis in mice. Nat. Commun. 9, 1-10 (2018).
T. Handa, et al., Caspase14 expression is associated with triple negative phenotypes and cancer stem cell marker expression in breast cancer patients. J Surg. Oncol. 116, 706-715 (2017).
R. Hrstka, et al., The pro-metastatic protein anterior gradient-2 predicts poor prognosis in tamoxifen-treated breast cancers. Oncogene 29, 4838-4847 (2010).
M. Pizzi, et al., Anterior gradient 2 overexpression in lung adenocarcinoma. Appl. Immunohistochem. Mol. Morphol. AIMM 20, 31-36 (2012).
H. Cho, A. B. Mariotto, L. M. Schwartz, J. Luo, S. Woloshin, When do changes in cancer survival mean progress? The insight from population incidence and mortality. J. Natl. Cancer Inst. Monogr. 2014, 187-197 (2014).
Y. M. Lo, et al., Rapid clearance of fetal DNA from maternal plasma. Am. J Hum. Genet. 64, 218-224 (1999).
M. A. Watson, T. P. Fleming, Mammaglobin, a mammary-specific member of the uteroglobin gene family, is overexpressed in human breast cancer. Cancer Res. 56, 860-865 (1996).
G. H. Lewis, et al., Relationship between molecular subtype of invasive breast carcinoma and expression of gross cystic disease fluid protein 15 and mammaglobin. Am. J Clin. Pathol. 135, 587-591 (2011).
R. Z. Liu, et al., A fatty acid-binding protein 7/RXRβ pathway enhances survival and proliferation in triple-negative breast cancer. J. Pathol. 228, 310-321 (2012).
A. Cordero, et al., FABP7 is a key metabolic regulator in HER2+ breast cancer brain metastasis. Oncogene 38, 6445-6460 (2019).
H. Zhang, et al., The proteins FABP7 and OATP2 are associated with the basal phenotype and patient outcome in human breast cancer. Breast Cancer Res. Treat. 121, 41-51 (2010).
J. Xiao, et al., Eight potential biomarkers for distinguishing between lung adenocarcinoma and squamous cell carcinoma. Oncotarget 8, 71759-71771 (2017).
M. Grageda, P. Silveyra, N. J. Thomas, S. L. DiAngelo, J. Floros, DNA methylation profile and expression of surfactant protein A2 gene in lung cancer. Exp. Lung Res. 41, 93-102 (2015).
Z. Zhang, et al., High expression of SLC34A2 is a favorable prognostic marker in lung adenocarcinoma patients. Tumour Biol. J. Int. Soc. Oncodevelopmental Biol. Med. 39, 1010428317720212 (2017).
F. Diehl, et al., Circulating mutant DNA to assess tumor dynamics. Nat. Med. 14, 985-990 (2008).
Liu M. C. et al., Sensitive and specific multi-cancer detection and localization using methylation signatures in cell-free DNA. Ann Oncol. 31(6), 745-59 (2020).
Anderson, N Leigh. “The clinical plasma proteome: a survey of clinical assays for proteins in plasma and serum.” Clinical chemistry vol. 56, 2 (2010): 177-85. doi:10.1373/clinchem.2009.126706.
Zehentner, Barbara K et al. “Mammaglobin as a novel breast cancer biomarker: multigene reverse transcription-PCR assay and sandwich ELISA.” Clinical chemistry vol. 50, 11 (2004): 2069-76. doi:10.1373/clinchem.2004.038687.

References and citations to other documents, such as patents, patent applications, patent publications, journals, books, papers, web contents, have been made throughout this disclosure. All such documents are hereby incorporated herein by reference in their entirety for all purposes.

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof. All references cited throughout the specification are expressly incorporated by reference herein.

The foregoing detailed description of embodiments refers to the accompanying drawings, which illustrate specific embodiments of the present disclosure. Other embodiments having different structures and operations do not depart from the scope of the present disclosure. The term “the invention” or the like is used with reference to certain specific examples of the many alternative aspects or embodiments of the applicants' invention set forth in this specification, and neither its use nor its absence is intended to limit the scope of the applicants' invention or the scope of the claims. This specification is divided into sections for the convenience of the reader only. Headings should not be construed as limiting of the scope of the invention. The definitions are intended as a part of the description of the invention. It will be understood that various details of the present invention may be changed without departing from the scope of the present invention. Furthermore, the foregoing description is for the purpose of illustration only, and not for the purpose of limitation.

While the present invention has been described with reference to the specific embodiments thereof, it should be understood by those skilled in the art that various changes may be made and equivalents may be substituted without departing from the true spirit and scope of the invention. In addition, many modifications may be made to adapt to a particular situation, material, composition of matter, process, process step or steps, to the objective, spirit and scope of the present invention. All such modifications are intended to be within the scope of the claims appended hereto.

1. A method of detecting cancer in a subject, the method comprising:
(a) measuring a plurality of target molecules in a biological fluid of the subject, wherein the plurality of target molecules are selected from polypeptides of Table 11; and
(b) detecting the cancer, wherein detecting the cancer comprises detecting one or more of the target molecules above a threshold level.

2. The method of claim 1, wherein the plurality of target molecules are selected from polypeptides of one or more of Tables 8 or 12-19.

3. The method of claim 1, wherein the plurality of target molecules are selected from at least 5, 10, 15, or 20 polypeptides of Tables 8, 11-14 or 17-19.

4. The method of claim 1, wherein the plurality of target molecules comprises a plurality of polypeptides from (i) Table 11; (ii) each of Tables 2, 5, and 12, (iii) each of Tables 3, 4, 6, and 13, (iv) Table 14, (v) Table 8, or (vi) Tables 18 and 19.

5. (canceled)

6. (canceled)

7. The method of claim 1, wherein the plurality of target molecules detected above a threshold are polypeptides selected from the group consisting of: ADAMTS15, AFP, AGR2, AIF1, ALPP, BPIFB2, CEACAM5, CGA, CHGA, CWC15, CXCL17, DXO, ERBB2, ERBB4, F7, FGFBP1, FGFR2, FKBPL, FOLR1, GDF15, GFRA1, GP2, INSL4, LAMA4, LEP, MIA, MMP12, PRSS8, SFRP1, SFTPA2, SPON1, TFF1, VTCN1, and WFDC2.

8. (canceled)

9. The method of claim 1, wherein the plurality of target molecules detected above a threshold are polypeptides selected from the group consisting of: CEACAM5, RHOV, SFTA2, SCGB1D2, IGF2BP1, SFTPA1, CA12, SFTPB, CDH3, MUC6, SLC6A14, HOXC9, AGR3, TMEM125, TFAP2B, IRX2, POTEKP, ARHGEF38, GPR87, LMX1B, ATP10B, NELL1, MUC21, SOX9, LINC00993, STMND1, ERVH48-1, SCTR, MAGEA3, MB, LEMD1, SIX4, and NXNL2.

10. The method of claim 1, wherein the plurality of target molecules comprise (a) polypeptides of one or more of Tables 11-14, and (b) one or more polypeptides of Tables 1-6.

11. (canceled)

12. The method of claim 1, wherein (i) the cancer is lung cancer, and (ii) the plurality of target molecules detected above a threshold are selected from polypeptides of Table 18, and optionally (iii) the plurality of target molecules detected above a threshold are selected polypeptides of one or more of WFDC2, CXCL17, MMP12, GDF15, or CEACAM5.

13. (canceled)

14. The method of claim 1, wherein (i) the cancer is breast cancer, and (ii) the plurality of target molecules detected above a threshold are selected from polypeptides of Table 19, and optionally (iii) the plurality of target molecules detected above a threshold are selected from polypeptides of one or more of ADAMTS15, LEP, ERBB2, ERBB4, or CGA.

15. (canceled)

16. The method of claim 1, wherein the plurality of target molecules comprises polypeptides of Table 16A or Table 16B

17. The method of claim 16, wherein the plurality of target molecules comprises: (a) polypeptides of Table 17, or (b) polypeptides selected from AGR3, CA12, CEACAM5, CXCL17, GP2, IL20, MMP7, TFF1, VTCN1.

18. (canceled)

19. The method of claim 1, wherein:
(a) the plurality of target molecules further comprises cell-free polynucleotides comprising (i) cell-free DNA (cfDNA) from genes encoding the polypeptides, and/or (ii) cell-free RNA (cfRNA) transcripts of the genes encoding the polypeptides; and
(b) detecting one or more of the target molecules above a threshold level comprises (i) detecting one or more of the polypeptides above a first threshold level, and (ii) for each of the polypeptides detected above the first threshold level, detecting a corresponding cell-free polynucleotide above a second threshold level.

20. The method of claim 19, wherein the cell-free polynucleotides comprise cfRNA or cfDNA.

21. (canceled)

22. The method of claim 20, wherein the cfDNA is methylated cfDNA.

23. (canceled)

24. The method of claim 19, wherein the measuring comprises sequencing the cell-free polynucleotides to produce sequence reads.

25. The method of claim 24, wherein the sequencing comprises: (i) whole transcriptome sequencing, (ii) sequencing cDNA molecules reverse transcribed from the cfRNA, or (iii) sequencing an enriched population of cfRNA or cfDNA.

26. (canceled)

27. (canceled)

28. (canceled)

29. (canceled)

30. The method of claim 1, wherein detecting one or more of the target molecules above a threshold level comprises (i) detection, (ii) detection above background, or (iii) detection at a level that is greater than a level of the one or more target molecules in subjects that do not have the cancer.

31. (canceled)

32. (canceled)

33. The method of claim 19, wherein the cell-free polynucleotides comprise cfRNA transcripts, and detecting one or more of the cfRNA transcripts above the second threshold level comprises:
(a) determining an indicator score for cfRNA transcript by comparing the expression level of each of the cfRNA transcript to an RNA tissue score matrix;
(b) aggregating the indicator scores for each cfRNA transcript; and,
(c) detecting the cancer when the indicator score exceeds a threshold value.

34. The method of claim 24, wherein detecting one or more of the cell-free polynucleotides above a threshold level comprises inputting the sequence reads into a machine learning or deep learning model.

35. The method of claim 34, wherein:
(a) the machine learning or deep learning model comprises logistic regression, random forest, gradient boosting machine, Näive Bayes, neural network, or multinomial regression; or
(b) the machine learning or deep learning model transforms the values of the one or more features to the disease state prediction for the subject through a function comprising learned weights.

36. (canceled)

37. The method of claim 1, wherein the cancer comprises:
(i) a carcinoma, a sarcoma, a myeloma, a leukemia, a lymphoma, a blastoma, a germ cell tumor, or any combination thereof;
(ii) a carcinoma selected from the group consisting of adenocarcinoma, squamous cell carcinoma, small cell lung cancer, non-small-cell lung cancer, nasopharyngeal, colorectal, anal, liver, urinary bladder, testicular, cervical, ovarian, gastric, esophageal, head-and-neck, pancreatic, prostate, renal, thyroid, melanoma, and breast carcinoma;
(iii) hormone receptor negative breast carcinoma or triple negative breast carcinoma;
(iv) a sarcoma selected from the group consisting of: osteosarcoma, chondrosarcoma, leiomyosarcoma, rhabdomyosarcoma, mesothelial sarcoma (mesothelioma), fibrosarcoma, angiosarcoma, liposarcoma, glioma, and astrocytoma;
(v) a leukemia selected from the group consisting of myelogenous, granulocytic, lymphatic, lymphocytic, and lymphoblastic leukemia; or
(vi) a lymphoma selected from the group consisting of: Hodgkin's lymphoma and Non-Hodgkin's lymphoma.

38. The method of claim 1, wherein detecting the cancer comprises determining a cancer stage, determining cancer progression, determining a cancer type, determining cancer tissue of origin, or a combination thereof.

39. The method of claim 1, further comprising selecting a treatment based on the cancer detected, and treating the subject with the selected treatment.

40. The method of claim 39, wherein the treatment comprises surgical resection, radiation therapy, or administering an anti-cancer agent.

41. (canceled)

42. A computer system for implementing one or more steps in the method of claim 1.

43. A non-transitory, computer-readable medium, having stored thereon computer-readable instructions for implementing one or more steps in the method of claim 1.
